SEQUENCE LISTING (4 sequences)

SEQ ID NO: 1 - DNA, 2048 bp, Listeria monocytogenes
taacgacgataaagggacagcaggactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatcgttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatcctgaaggtaacgaaattgttcaacataaaaactggagcgaaaacaataaaagcaagctagctcatttcacatcgtccatctatttgccaggtaacgcgagaaatattaatgtttacgctaaagaatgcactggtttagcttgggaatggtggagaacggtaattgatgaccggaacttaccacttgtgaaaaatagaaatatctccatctggggcaccacgctttatccgaaatatagtaataaagtagataatccaatcgaataattgtaaaagtaataaaaaattaagaataaaaccgcttaacacacacgaaaaaataagcttgttttgcactcttcgtaaattattttgtgaagaatgtagaaacaggcttattttttaatttttttagaagaattaacaaatgtaaaagaatatctgactgtttatccatataatataagcatatcccaaagtttaagccacctatagtttctactgcaaaacgtataatttagttcccacatatactaaaaaacgtgtccttaactctctctgtcagattagttgta

SEQ ID NO: 2 - protein, 529 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 3 - DNA, 276 bp, Enterobacteria phage phiX174
atggtacgctggactttgtgggatacgctagctttcctgctcctgttgagtttattgctgccaagcttgctgatcatgttcatcccgtcgacattcaaacggccggtgagctcatggaaggcgctgaatttacggaaaacattattaatggcctcgagcgtccggttaaagccgctgaactgcagccggttaccttgcgtgtacgcgcaggaaacactgacgttcttactgacgcagaagaaaacgtgcgtcaaaaattacgtgcagaaggagtga

SEQ ID NO: 4 - protein, 91 aa, Enterobacteria phage phiX174
MVRWTLWDTLAFLLLLSLLLPSLLIMFIPSTFKRPVSSWKALNLRKTLLMASSVRLKPLNCSRLPCVYAQETLTFLLTQKKTCVKNYVRKE

(19) United States
(12) Patent Application Publication
(10) Pub. No.: US 2017/0348402 A1
(43) Pub. Date: Dec. 7, 2017

(54) SYSTEM AND METHOD FOR DELIVERING GENETIC MATERIAL OR PROTEIN TO CELLS

(71) Applicant: The Research Foundation for The State University of New York, Amherst, NY (US)

(72) Inventors: Blaine A. Pfeifer, Buffalo, NY (US); Charles Jones, North Tonawanda, NY (US)

(21) Appl. No.: 15/329,927

(22) PCT Filed: Jul. 30, 2015
(86) PCT No.: PCT/US2015/042868

§ 371 Date: Jan. 27, 2017

Related U.S. Application Data
(60) Provisional application No. 62/030,815, filed on Jul. 30, 2014.

(51) Int. Cl. A61K 39/00; A61K 48/00; C12N 15/88

(52) U.S. Cl. CPC A61K 39/0011; C12N 15/88; A61K 48/0025; A61K 2039/52

(57) ABSTRACT

It has been established that bacterial hybrid vectors including prokaryote cells modified by the addition of cationic polymers to the outer surface of the cell can selectively deliver exogenous cargos, such as nucleic acids, polypeptides and small molecules to an eukaryotic cell, such as an antigen presenting cell. Compositions and methods for the delivery and expression of nucleic acids and polypeptides to eukaryotic cells are described. The bacterial hybrid vectors include one or more cationic polymers that enhance uptake by antigen presenting cells. The hybrid bacterial vectors include expression vectors that express one or more factors to enhance lysosomal escape and cytosolic delivery of cargo. The vectors are useful as adjuvants to stimulate and/or induce immune responses to any desired antigen, to develop a protective immune response in a subject.

### CROSS-REFERENCE TO RELATED APPLICATIONS

This application claims priority to U.S. Provisional Application No. 62/030,815 entitled “System and method for delivering genetic material or protein to cells” filed Jul. 30, 2014.

### STATEMENT REGARDING FEDERALLY SPONSORED RESEARCH OR DEVELOPMENT

This invention was made with Government Support under NIH grant No. AI088485 awarded to Blaine A. Pfeifer by the National Institutes of Health. The Government has certain rights in the invention.

### REFERENCE TO SEQUENCE LISTING

The Sequence Listing submitted Jul. 30, 2015 as a text file named “UB-R-6878PCT_ST25.txt,” created on Jul. 29, 2015, and having a size of 9,000 bytes is hereby incorporated by reference.

### FIELD OF THE INVENTION

The field of the invention is generally related to molecular delivery systems, and more specifically, to compositions, devices, and methods for the delivery of protein or genetic material such as DNA and RNA into eukaryotic or prokaryotic cells to treat or prevent diseases and/or conditions.

### BACKGROUND OF THE INVENTION

Increasing prevalence of antibiotic resistance and the specter of living in a “post-antibiotic era” has raised concerns from the World Health Organization that alternative effective means for addressing infectious diseases are necessary (Antimicrobial resistance: global report on surveillance, World Health Organization, 2014). However, only 27 human diseases are recognized by the U.S. Center of Disease Control as preventable by vaccination despite centuries of development. Furthermore, current approaches often rely on technology that is limited in rapid production capability and associated engineering parameters to influence the type, duration, and potency of an immune response.

Gene therapy has emerged as an alternative approach to the classical vaccine paradigm by utilizing the delivery of specific therapeutic cargo for the purpose of altering gene expression. The delivery of DNA requires the assistance of a vector to facilitate the process of gene expression within immune system sentinels termed antigen presenting cells (APCs). In this context, the delivery of nucleic acids is accomplished by a variety of biological or synthetic vectors. Through specific engineering tools, each vector is designed to influence antigen presenting cell (APC) gene expression levels to modulate an immune response towards the generation of antigen reactivity and memory. Vector-mediated gene delivery efficacy is strongly correlated with overcoming APC barriers such as cellular uptake, phagosomal/lysosomal escape, nucleic acid un-packaging, nuclear translocation (excluding RNA-based therapeutics), and sustained gene expression. To be clinically relevant, vectors must also exert minimal to no cytotoxicity.

Small interfering RNA (siRNA) has also been used to interfere with genetic expression. However, RNA interference also faces a number of delivery issues that have limited its overall success, especially for in vivo applications. A number of barriers exist that a successful siRNA therapeutic must overcome. First, macrophages and cells of the reticulo-endothelial system see the siRNA complex as a foreign entity and attempt to degrade and eliminate it. Second, getting the siRNA to the proper organ/cell of interest is a major challenge. Third, once inside the cell, it must be able to escape the endosome. Finally, once those obstacles have been overcome, it must be potent enough to accomplish knockdown of the target sequence.

The ability to deliver nucleic acids and proteins to mammalian cells has been demonstrated with biomaterial-based nanoparticles. Of the biomaterial vectors, cationic polymers (CPs) facilitate uptake by generalized endocytosis mechanisms and instigate lysosomal escape by the “proton sponge effect”. However, “Polyplexes” of cationic polymers and nucleic acids are often destabilized by salts and serum components, and can break apart or aggregate in physiological fluids (A1-Dosari, et al. Nonviral gene delivery: principle, limitations, and recent progress. AAPS J. 11, 671-681 (2009)) (Tros de Ilarduya, et al. Gene delivery by lipoplexes and polyplexes. Eur. J. Pharm. Sci. 40, 159-170 (2010)). Thus, these delivery vehicles are inefficient when compared to biological delivery options (such as viral vectors). Further, many cationic vectors exhibit cytotoxicity, (Tros de Ilarduya, et al. Eur. J. Pharm. Sci. 40, 159-170 (2010); Gao, et al. Biomaterials 32, 8613-8625 (2011);Feigner, et al. J. Biol. Chem. 269, 2550-2561 (1994); Kafil, et al. BioImpacts 1, 23-30 (2011); Lv, et al. J Contr. Rel. 114, 100-109 (2006)). Using CPs also requires direct addition, usually in substantial quantities, of the genetic material or protein antigens needed for an immune response which can be a costly approach to vector preparation.

Nucleic acid and protein delivery to mammalian cells has also been demonstrated through the use of bacteria. Bacterial vectors provide an orthogonal set of engineering tools to influence gene delivery. As an example, Escherichia coli has been used for delivery of genetic and polypeptide-based cargo to the interior of eukaryote cells in vitro and in vivo (Jones C H, et al. Mol Pharm 10(11):4301-4308(2013); Larsen M D, et al. Gene Ther 15(6):434-442. (2008); Castagliuolo I, et al. Gene Ther 12(13):1070-1078 (2005); Chart H. J Appl Microbiol 89(6):1048-1058 (2000); Parsa S, J Biotechnol 137(1-4):59-64. (2008); Critchley R J, et al. Gene Ther 11(15):1224-1233 (2004); Laner A, et al. Gene Ther 12(21):1559-1572 (2005); Cheung W, et al., Bioeng Bugs 3(2):86-92(2012); Xiang S, Nat Biotechnol, 24(6):697-702 (2006); Radford, et al., Gene Ther. 9(21):1455-1463 (2002); Higgins, et al., Mol Microbiol 31(6):1631-1641 (1999)). However, these bacterial vectors are hampered by poor uptake and delivery to eukaryote cells, as well as concerns associated with toxicity and safety.

Therefore, it is an object of the invention to provide compositions and methods of use thereof for enhanced uptake of nucleic acids, proteins and small molecules by antigen presenting cells that exert minimal or no cytotoxicity.

It is also an object of the invention to provide compositions and methods of use thereof to enhance phagosomal/lysosomal escape and nuclear translocation of nucleic acids, proteins and other small molecules delivered to antigen presenting cells.

It is a further object of the invention to provide compositions, methods, and devices for sustained expression of exogenous genes by antigen presenting cells.

### SUMMARY OF THE INVENTION

It has been established that recombinant bacterial vectors can be engineered as vehicles to enhance delivery of prophylactic and/or therapeutic vaccines, gene therapy, antisense nucleic acids, RNA interference, and tissue engineering reagents. Prokaryotic cells modified by association with a cationic polymer outer coating are provided as vectors for the delivery of antigen and other molecules to eukaryotic “target” cells. The modified “hybrid” bacterial vectors combine innate and engineered features of bacteria and cationic polymers to enhance gene delivery. The hybrid bacterial vectors can be used for in vivo and/or in vitro delivery of nucleic acids and peptides to eukaryote cells. The hybrid bacterial vectors are non-toxic to eukaryote cells.

Hybrid bacterial vectors for delivery of exogenous polypeptides and nucleic acids into eukaryote cells include one or more biodegradable cationic polymers associated with the outer surface of the prokaryotic cell in an amount sufficient to impart a positive charge to the prokaryotic cell, and one or more nucleic acid plasmids. The one or more nucleic acid plasmids include one or more genes encoding exogenous polypeptides and nucleic acids and one or more pore-forming polypeptides. Exemplary pore-forming polypeptides include pore-forming proteins, such as lysteriolysin O (LLO) and enzymes such as endolysins. The pore forming polypeptides can facilitate egress from the lysosome and/or endosome of an eukaryotic cell following uptake of the hybrid bacterial vector by an eukaryotic cell.

Prokaryote cells for use in the hybrid bacterial vectors can be live, un-attenuated bacteria; live, attenuated bacteria; and inactivated bacteria. For example, the prokaryote cell can be a strain of Escherichia coli, preferably a strain of Escherichia coli that is non-pathogenic in humans. Exemplary Escherichia coli strains include Escherichia coli RR1; Escherichia coli LE392; Escherichia coli B, Escherichia coli 1776 (ATCC No. 31537); Escherichia coli W3110 (F-, lambda-, prototrophic, ATCC No. 273325); Escherichia coli strain YWT7-hly; and Escherichia coli K.

Exemplary biodegradable cationic polymers for use in hybrid bacterial vectors include poly(beta-amino esters); aliphatic polyesters; polyphosphoesters; poly(L-lysine) containing disulfide linkages; poly(ethylenimine); disulfide-containing polymers such as DTSP or DTBP crosslinked PEI; PEGylated PEI crosslinked with DTSP; Crosslinked PEI with DSP; Linear SS-PEI; DTSP-Crosslinked linear PEI; branched poly(ethylenimine sulfide) (b-PEIS). A preferred biodegradable cationic polymer is synthesized by conjugate addition of Neopentyl glycol diacrylate to 2-Amino-1,3-propanediol. In some embodiments, the biodegradable cationic polymer is modified by the addition of polyethylene glycol. Typically, the biodegradable cationic polymer has a charge density of between −50 and −30 mV, inclusive, and a molecular weight of between 500 Da and 20.000 Da, inclusive, for example, approximately 1,000 Da to 10,0000 Da, inclusive. A preferred cationic polymer has a molecular weight of approximately 5000-6000 Da.

The hybrid bacterial vectors can optionally include one or more functional groups, such as targeting elements, immune-modulatory elements, chemical groups, biological macromolecules, or combinations thereof. For example, the hybrid bacterial vectors can include one or more immune-modulatory elements such as CRM197 (diphtheria toxin), outer membrane protein complex from Neisseria meningitides, viral hemagglutinin and neuraminidase.

Exemplary targeting elements include ligands such as Fc gamma RIIB, DCIR, DC-SIGN, Dectin-1, CLEC9A, Langerin, CD11c, CD163, FC gamma RIIB, and Her2. In certain embodiments the biodegradable cationic polymer is modified to include an antibody, an antibody fragment, or proteins having the binding specificity of an antibody.

Preferably the biodegradable cationic polymer is modified with one or more targeting elements that mediate specific uptake by professional antigen presenting cells (APC). APC can include dendritic cells and macrophage cells. Exemplary targeting elements that mediate specific uptake by APC include ligands for receptors at the surface of APC. An exemplary receptor is the CD206 mannose-binding protein. In certain embodiments the biodegradable cationic polymer is modified by addition of one or more mannose moieties.

In some embodiments the bacterial hybrid vectors constitutively express one or more pore-forming proteins that can assist egress from the lysosome of eukaryotic cells. A preferred pore-forming protein is the listeriolysin O protein.

In some embodiments the bacterial hybrid vectors express one or more enzymes that lead to disruption of the bacterial cell wall. A preferred enzyme is the lethal lysis LyE gene of bacteriophage ΦX174.

The hybrid bacterial vectors include one or more nucleic acid plasmids including a promoter, an exogenous nucleic acid sequence downstream of and operably linked to the promoter, a transcription terminator downstream of and operably linked to the exogenous nucleic acid sequence, and an origin of replication. The promoter and transcription teiminator can be of eukaryotic or prokaryotic origin. Typically, the promoter is an inducible promoter. The exogenous nucleic acid sequence can encode a ribozyme, enzyme, peptide, structural protein, structural RNA, shRNA, siRNA, miRNA, transcription factor, signaling molecule, or a combination thereof.

Adjuvants including hybrid bacterial vectors and one or more antigens are also provided. In some embodiments the hybrid bacterial vector expresses the antigenic polypeptide. In other embodiments the hybrid bacterial vector delivers one or more genes encoding the antigen to the antigen presenting cells of a subject. In certain embodiments, the expression of the antigen is restricted to a specific cellular location within the bacterial cell of the hybrid vector. For example, the antigen may be expressed in the cytoplasm, the periplasm, the bacterial surface, or combinations thereof. In other embodiments, the antigen is secreted from the bacterial cell.

Pharmaceutical compositions including the hybrid bacterial vectors and a pharmaceutically acceptable excipient are also described. Excipients suitable for administration via the oral, nasal, ocular, rectal, intramuscular, intraperitoneal, pulmonary, epidermal and intradermal route are provided. Pharmaceutical compositions including one or more additional therapeutic, prophylactic or diagnostic agents are also provided.

Methods for inducing or stimulating an immune response to an exogenous antigen in the antigen presenting cells of a subject are also provided. Typically, the methods include administering to the subject pharmaceutical compositions including the hybrid bacterial vectors and a pharmaceutically acceptable excipient in an amount sufficient to induce an immune response in the antigen presenting cells of the subject. Exemplary antigen presenting cells include dendritic cells, neutrophils and macrophages. Exemplary antigens include viral antigens, bacterial antigens, protozoan antigens, fungal antigens, nematode antigens and cancer antigens. In some embodiments a hybrid bacterial vector includes more than one antigen.

Methods for delivery of exogenous polypeptides and nucleic acids into an eukaryotic cells are also provided. The methods can include contacting the eukaryote cell with one or more hybrid bacterial vectors in an amount and concentration effective to facilitate uptake of the hybrid bacterial vectors by the eukaryote cell. Preferably, the hybrid bacterial vector causes minimal or no toxicity in the eukaryote cell. Preferably, the multiplicity of infection of the hybrid bacterial vector is optimized for uptake by the eukaryote cell.

### BRIEF DESCRIPTION OF THE DRAWINGS

FIG. 1A is a diagram showing the reaction of the addition of diacrylate and primary amine by conjugate Michael addition to form base polymers. FIG. 1B is a schematic representative of high-throughput screening procedure for diacrylate and primary amine conjugates.

FIGS. 2A-2F are histograms. FIGS. 2A-2C show luminescence/μg protein for bacterial strains 1-5, each at concentrations of 0, 0.1, 0.25, 0.5 and 1 mg/ml, respectively, as well as fugene only (1 mg/ml; control) and D9 polyplex only (1 mg/ml; control) at a multiplicity of infection of 1:1 (FIG. 2A); 10:1 (FIG. 2B); and 100:1 (FIG. 2C), respectively. FIG. 2D shows % RAW264.7 relative to untreated control at dosages of 0, 0.1, 0.25, 0.5 and 1 mg/ml D9, respectively, at a multiplicity of infections (MOI) of 1:1; 10:1; and 100:1. FIG. 2E shows Nitric Oxide (NO) concentration (μM) at dosages of 0, 0.1, 0.2, 0.3, 0.4, 0.5, 0.6, 0.7, 0.8, 0.9 and 1 mg/ml, respectively, at a multiplicity of infection of 1:1; 10:1; and 100:1. FIG. 2F shows luminescence/μg protein at dosages of YWT7-hly/pCMV-Luc (S1; control), D9 Polyplex (Control), as well as D9 at 0, 0.1, 0.2, 0.3, 0.4, 0.5, 0.6, 0.7, 0.8, 0.9 and 1 mg/ml, respectively, for each of S1:D9 hybrid vector, S1+D9, S1:D9 Polyplex hybrid and YWT7-hly:D9 vector polyplex hybrid.

FIGS. 3A-3D are histograms. FIG. 3A shows bacterial number (CFU) of YWT7-hly/pRSET-EmGFP (grey) and YWT7-hly/pRSET-EmGFP:D9 (0.4. mg/ml) Hybrid (black), respectively, for multiplicity of infection of 1:1, 10:1 and 100:1. FIG. 3B shows zeta potential (mV) for D9 alone (control), YWT7-hly (control), as well as D9 at a dosage of 0.1, 0.2, 0.3, 0.4, 0.5, 0.6, 0.7, 0.8, 0.9 and 1 mg/ml, respectively. FIG. 3C shows bacteria number (CFU) for D9 at a dosage of 0.1, 0.2, 0.3, 0.4, 0.5, 0.6, 0.7, 0.8, 0.9 and 1 mg/ml, respectively, for 0 s (black) and 5 s (white), respectively. FIG. 3D shows % hydrophobicity for YWT7-hly (control), as well as D9 at dosage of 0.1, 0.2, 0.3, 0.4, 0.5, 0.6, 0.7, 0.8, 0.9 and 1 mg/ml, respectively.

FIG. 4A is a line graph showing optical density at 600 nanometers (OD₆₀₀) over induction time (minutes) for BL21(DE3) cells in the presence of 100 μM (x); 500 μM (*); and 1000 μM (•) IPTG, respectively, as well as BL21(DE3)/pCYC-LyE cells in the presence of 100 μM (♦); 500 μM (▪); and 1000 μM (▴) IPTG, respectively. FIGS. 4B-4D are histograms.

FIG. 4B shows bacterial number (CFU) over induction times of 60, 90, 120, 150 and 180 minutes for each of BL21(DE3) cells (black) and BL21(DE3)/pCYC-LyE cells (white), respectively. FIG. 4C shows luminescence/μg protein for each of YWT7-hly/pCMV-Luc (white) and YWT7-hly/pCMV-Luc/pCYC-LyE (black), at a multiplicity of infection (MOI) of 1:1; 10:1; 100:1; and 1000:1, respectively. FIG. 4D shows % viability of RAW264.7 cells relative to untreated control cells for YWT7-hly (white) YWT7-hly/pCMV-Luc/pCYC-LyE (black), at a multiplicity of infection (MOI) of 1:1; 10:1; 100:1; 500:1; 1000:1; 1500:1 and 2000:1, respectively. *Statistical significance (95% confidence) for samples with and without pCYC-LyE compared at induction time points (FIG. 4B) or MOIs (FIGS. 4C and 4D).

FIGS. 5A-5E are histograms. FIGS. 5A-5C show number of bacterial cells (CFU) over concentration of Mannose Addition (mg/ml) for each of YWT7-hly/pRSET-EmGFP (grey); YWT7-hly/pRSET-EmGFP:D9 (0.4 mg/ml) Hybrid (black); and YWT7-hly/pRSET-EmGFP:D9-Man (0.4 mg/ml) Hybrid (hashed), respectively, for multiplicity of infection of 1:1 (FIG. 5A), 10:1 (FIG. 5B) and 100:1 (FIG. 5C). FIGS. 5D-5F show luminescence/μg protein of YWT7-hly/pCMV-Luc (S1; control); S1-LyE; S1:D9; S1-LyE:D9; S1:D9-Man; and S1-LyE:D9-Man, respectively, for multiplicity of infection of 1:1 (FIG. 5D), 10:1 (FIG. 5E) and 100:1 (FIG. 5F). *Statistical significance (95% confidence) indicating reduced bacterial uptake at each mannose concentration.

FIGS. 6A-6B are histograms. FIG. 6A shows Anti-OVA IgG1 (μg/ml) at day 14 (black) and Day 21 (white) for each of pDNA+Adj.; Protein+Adj.; S1:D9 Hybrid S.Q. (1×10⁵); S1:D9 Hybrid I.P. (1×10⁵); S1:D9 Hybrid S.Q. (1×10⁷); and S1:D9 Hybrid I.P. (1×10⁷), respectively. FIG. 6B shows Anti-OVA IgG1 (μg/ml) per μg antigen at day 14 (black) and Day 21 (white) for each of pDNA+Adj.; Protein+Adj.; S1:D9 Hybrid S.Q. (1×10⁵); S1:D9 Hybrid I.P. (1×10⁵); S1:D9 Hybrid S.Q. (1×10⁷); and S1:D9 Hybrid I.P. (1×10⁷). *Statistical significance (95% confidence) compared to pDNA (FIG. 6A; respective time points) or pDNA and protein (FIG. 6B; respective time points) OVA controls.

FIGS. 7A-7B are schematic representations showing the formation (FIG. 7A) and biological activity (FIG. 7B) of the bacterial hybrid vectors. respectively. FIG. 7A demonstrates addition of positively charges cationic polymers to the outer surface of negatively charged cells provides a positively charged vector. FIG. 7B illustrates vector uptake by phagocytosis (1); phagosome acidification (2) leads to degradation of vectors, releasing nucleic acid contents into the phagosome; rupture of the phagosome by endolysin enzymes (3) releases nucleic acids into the cytoplasm (4); resulting in translocation to the nucleus, giving rise to biological effector functions, such as immune modulation (5).

FIGS. 8A-8G are histograms showing % gene delivery relative to untreated control for each of 92 different poly(beta-amino esters); A1-A1 (FIG. 8A); B1-B13 (FIG. 8B); C1-C13 (FIG. 8C); D1-D13 (FIG. 8D); E1-E13 (FIG. 8E); F1-F13 (FIG. 8F); and G1-G13 (FIG. 8G), respectively, each at a concentration of 0.1 mg/ml (black); 1.0 mg/ml (white); and 10 mg/ml (hashed).

FIGS. 9A-9G are histograms showing zeta potential (mV) for each of 92 different poly(beta-amino esters); A1-A1 (FIG. 9A); B1-B13 (FIG. 9B); C1-C13 (FIG. 9C); D1-D13 (FIG. 9D); E1-E13 (FIG. 9E); F1-F13 (FIG. 9F); and G1-G13 (FIG. 9G), respectively, each at a concentration of 0 mg/ml (grey); 0.1 mg/ml (hashed); and 10 mg/ml (white), as well as for the polymer alone (black).

FIGS. 10A-10C are histograms showing luminescence/μg protein for each of Fugene 6 (control), YWT7-hly/pCMV-Luc, as well as each of the top 20 poly(beta-amino esters) from the initial screen, including A5, A11, B6, B9, B11, C2, C5, C9, C10, C13, D1, D7, D9, D13, E1, E7, F1, F7, F11 and G2, respectively, each at a concentration of 0.1 mg/ml (white); 0.25 mg/ml (hashed); 0.5 mg/ml (light grey) and 1.0 mg/ml (dark grey), as well as for the polyplexes alone (black).

FIGS. 11A-11B are histograms. FIG. 11A shows % RAW264.7 cells relative to untreated control cells for D9 added at a dosage of 0.1, 0.25, 0.5, and 1 mg/ml, respectively. FIG. 11B shows Nitric Oxide (NO) concentration (μM) for dosages of D9 of 0, 0.1, 0.2, 0.3, 0.4, 0.5, 0.6, 0.7, 0.8, 0.9 and 1 mg/ml, respectively, as well as for 1:1 BL21(DE3) cells and No treatment.

FIG. 12 is a schematic showing (i) S1:D9 polyplex and YWT7-hly:D9 polyplex hybrid formation and (ii) S1+D9 mixing.

FIGS. 13A-13B are histograms showing % viable RAW264.7 cells relative to untreated control cells for YWT7-hly (black) and YWT7-hly/pCMV-Luc/pCYC-LyE (white) at a multiplicity of infection (MOI) of 1:1; 10:1; 100:1; 500:1; 1000:1; 1500:1 and 2000:1, respectively, in the presence of 100 μM IPTG (FIG. 13A) and 500 μM IPTG (FIG. 13B).

FIGS. 14A-14D are diagrams showing the reaction schemes for the addition of Neopentyl glycol diacrylate (D) and 2-Aminopropane-1,3-diol(9) to form Acrylate-terminated poly(neopentyl glycol diacrylate-co-2-amino1,3-propanediol) (D9-Ac) (FIG. 14A); the addition of D9-Ac and Ethylenediamine with DMSO to form D9-am (FIG. 14B); the conversion of D-mannose with allyl alcohol to Ally-a-D-mannopyranoside (ADM) (FIG. 14C); and the addition of D9-am and ADM with DMSO to form D9-Man (FIG. 14D).

FIG. 15A is a diagram of gel-permeation chromatographs of D9 and D9-man, superimposed over Retention volume (ml). FIGS. 15B-15C are 500 MHz 1H NMR spectra of D9 and D9-man, respectively.

FIGS. 16A-16D are histograms. FIG. 16A shows Sheep red blood cell (RBC) hemolysis (% RBC Lysis (pH 7.4)) over PBS, TRITON-X100, BL21(DE3), CPLA-26, CPLA-54, PEG-b-CPLA-20, PEG-b-CPLA-50, S1:C26, S1:C54, S1:PC20 and S1:PC50, respectively, for dosages of 0.1, 0.25, 0.5, 1.0 and 3.0 mg/ml. FIG. 16B shows hybrid membrane shear studies (with 5 s sonication), depicted as No. colony forming units (CFU) over S1-PBS, S1:Cs6, S1:C54, S1:PC20 and S1:PC50, respectively, for dosages of 0.1, 0.25, 0.5, 1.0 mg/ml. FIGS. 16C and 16D show protein release measured by Absorbance (A260) for each of PBS, Polymixin B, CPLA-26, CPLA-54, PEG-b-CPLA-20 and PEG-b-CPLA-50, respectively, for dosages of 0.1, 0.25, 0.5, 1.0 mg/ml. *Statistical significance (95% confidence) compared to respective polymers (FIG. 16A) or bacterial (FIG. 16B) and PBS (FIGS. 16C and 16D) controls. Abbreviations: CPLA-26: C26; CPLA-54: C54; PEG-b-CPLA-20: PC20; PEG-b-CPLA-50: PC50.

FIGS. 17A-17D are line graphs. FIGS. 17A and 17C show % Hydrophobicity (17A) and Fluorescence (450 nm) (17C) for each of the polymers D3A5; D4A3; D5A4; D3mA4; D4A4; D5A5; D3mA5; D4A5; D6A5 each with polymer doses between 0.1 and 2.0 mg/ml, respectively, where the bacteria alone have a hydrophobicity of 5.63% and the positive control has a Fluorescence (450 nm) of 42,356 nm. FIGS. 17B and 17D show % Hydrophobicity (17B) and Fluorescence (450 nm) (17D) for each of the polymers D3A5-Man; D3mA5-Man; D4A4-Man; D5A4-Man; D6A5-Man; D3mA4-Man; D4A3-Man; D4A5-Man and D5A5-Man, each with polymer doses between 0.1 and 2.0 mg/ml, respectively, where the bacteria alone have a hydrophobicity of 5.63% and the positive control has a Fluorescence (450 nm) of 42,356 nm.

FIGS. 18A-18D are line graphs. FIGS. 18A and 18C show Zeta potential (mV) for each of the polymers D3A5; D4A3; D5A4; D3mA4; D4A4; D5A5; D3mA5; D4A5; D6A5 (18A) and D3A5-Man; D3mA5-Man; D4A4-Man; D5A4-Man; D6A5-Man; D3mA4-Man; D4A3-Man; D4A5-Man and D5A5-Man (18B), each with polymer doses between 0.1 and 2.0 mg/ml in 25 mM NaOAc (pH 5.15), respectively, with the bacteria alone have a zeta potential of −18.34 mV. FIGS. 18B and 18D show Zeta potential (mV) for each of the polymers D3A5; D4A3; D5A4; D3mA4; D4A4; D5A5; D3mA5; D4A5; D6A5 (18B) and D3A5-Man; D3mA5-Man; D4A4-Man; D5A4-Man; D6A5-Man; D3mA4-Man; D4A3-Man; D4A5-Man and D5A5-Man (18D), respectively, each with polymer doses between 0.1 and 2.0 mg/ml in PBS (pH 7.4), respectively, with the bacteria alone have a zeta potential of −20.65 mV.

FIGS. 19A-19D are line graphs. FIG. 19A shows % Hydrophobicity over polymer dose (mg/ml) for each of the polymers P1-P14 with the bacteria alone having a hydrophobicity of 5.63%. FIG. 19B shows has a Fluorescence (450 nm) over polymer dose (mg/ml) for each of the polymers P1-P14, respectively, with a positive control of 42,356 nm. FIG. 19C shows Zeta potential (mV) for each of the polymers P1-P14 each with polymer doses between 0.1 and 2.0 mg/ml in 25 mM NaOAc (pH 5.15), respectively, with the bacteria alone have a zeta potential of −18.34 mV. FIG. 19D shows Zeta potential (mV) for each of the polymers P1-P14, each with polymer doses between 0.1 and 2.0 mg/ml in PBS (pH 7.4), respectively, with the bacteria alone have a zeta potential of −20.65 mV.

FIGS. 20A-20B are histograms, showing Luminescence/μg protein (FIG. 20A) and % viability (FIG. 20B) for each of FuGene HD (control); JET-PEI; YWT7-hly/pCMV-Luc; D3mA4; D3mA4-Man; D3A5; D3A5-Man; D3mA5; D3mA5-Man; D4A3; D4A3-Man; D4A4; D4A4-Man; D4A5; D4A5-Man; D5A4; D5A4-Man; D5A5; D5A5-Man; D6A5; and D6A5-Man, respectively, for dosages of 0.25, 0.5, 0.75, and 1.0 mg/ml.

FIGS. 21A-21B are histograms, showing Luminescence/μg protein for each of FuGene HD (control); JET-PEI; YWT7-hly/pCMV-Luc; and P1-P14, respectively, at dosages of 0.25; 0.5; 0.75; and 1.0 mg/ml (FIG. 21A) and in the presence of no inhibitor; 1000 μM Mannose; 50% FBS; and 1000 μM Mannose plus 50% FBS (FIG. 21B).

FIGS. 22A-22B are schematic representations of hybrid vector formulation and assembly. FIG. 22A is a diagram showing the layout of a normal cell wall of a Gram-negative bacterium. FIG. 22B is a diagram showing how the proposed hybrid formation model proceeds in four steps. First, polymer is adsorbed to the bacterial surface through charge-charge interaction (Step 1). Afterwards, the polymer diffuses slowly through the outer membrane (OM) while simultaneously compromising the structural integrity (Step 2). In the latter steps, the polymer chains diffuse slowly through the periplasmic space (Step 3) before subsequent integration and diffusion through the bacterial inner membrane (IM; Step 4).

FIGS. 23A-23D are histograms showing % Gene delivery of hybrid vectors relative to the control (sl) bacterial strain at various polymer doses formulated using CPLA-26 (FIG. 23A); CPLA-54 (FIG. 23B); PEG-b-CPLA-20 (FIG. 23C); and PEG-b-CPLA-50 (FIG. 23D). *Statistical significance (95% confidence) compared to Strain 1 (i.e., the 100% value). @Statistical significance (95% confidence) compared to hybrid vector prepared using respective nonPEGylated CPLA polymer. The table presented in FIG. 23B provides values (luminescence per μg protein) for raw gene delivery of bacterial vectors (at various MOIs), commercial controls, and CPLA polyplexes (polymer complexed to pDNA), respectively.

FIGS. 24A-24B are histograms showing % Gene delivery of hybrid vectors relative to the control (s 1) bacterial strain at various % FBS for different polymer doses (0.25; 0.5; 0.75; and 1.0 mg/ml) formulated using CPLA-26 (FIG. 24A) and PEG-b-CPLA-50 (FIG. 24B). *Statistical significance (95% confidence) compared to S1 transfection in 10% FBS. @Statistical significance (95% confidence) compared to hybrid vector prepared using respective nonPEGylated CPLA polymer.

FIGS. 25A-25D are histograms showing Cytotoxicity of RAW264.7 (% viability relative to untreated controls) incubated with hybrid vectors at various polymer doses (0.25; 0.5; 0.75; and 1.0 mg/ml) formulated using CPLA-26 (FIG. 25A) and CPLA-54 (FIG. 25B); PEG-b-CPLA-20 (FIG. 25C); and PEG-b-CPLA-50 (FIG. 25D), respectively, each at varied MOI (1:1, white bar; 10:1, black bar; and 100:1, hashed bar). *Statistical significant (95% confidence) decreases in viability of CPLA-54 hybrids compared to CPLA-26 hybrids. @Statistical significance (95% confidence) compared to hybrid vector prepared using respective nonPEGylated CPLA polymer.

FIG. 26 is a line graph showing Time (h) of survival of test animals following challenge for subject immunized with Sham (control); CFA/IFA, 100 μg; Plasmid; and Bacteria expressing the antigen in the Cytoplasm; Periplasm; Surface; and Excreted, respectively.

### DETAILED DESCRIPTION OF THE INVENTION

### I. Definitions

As used herein the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered. The effect of the effective amount can be relative to a control. Such controls are known in the art and discussed herein, and can be, for example the condition of the subject prior to or in the absence of administration of the drug, or drug combination, or in the case of drug combinations, the effect of the combination can be compared to the effect of administration of only one of the drugs.

The term “Inhibit” or other forms of the word such as “inhibiting” or “inhibition” means to hinder or restrain a particular characteristic. It is understood that this is typically in relation to some standard or expected value, i.e., it is relative, but that it is not always necessary for the standard or relative value to be referred to. For example, “inhibits expression” means hindering, interfering with or restraining the expression or activity of a gene relative to a standard or a control. “Inhibits activity” can also mean to hinder or restrain the synthesis, expression or function of the gene product, such as a protein, relative to a standard or control.

The terms “Treatment” or “treating” means to administer a composition to a subject or a system with an undesired condition (e.g., a genetic disorder). The condition can include a disease. “Prevention” or “preventing” means to administer a composition to a subject or a system at risk for the condition. The condition can be a predisposition to a disease. The effect of the administration of the composition to the subject (either treating and/or preventing) can be, but is not limited to, the cessation of a particular symptom of a condition, a reduction or prevention of the symptoms of a condition, a reduction in the severity of the condition, the complete ablation of the condition, a stabilization or delay of the development or progression of a particular event or characteristic, or minimization of the chances that a particular event or characteristic will occur.

The term “host cell” refers to prokaryotic and eukaryotic cells into which a recombinant expression vector can be introduced.

The term “specifically binds” (i.e., to a desired “target”) refers to a binding reaction which is determinative of the presence of the molecule in the presence of a heterogeneous population of other biologics. A “target” is the desired recipient or host of the targeted entity, such as a binding partner for a ligand, or a cell, or group of cells. For example, the target can be an eukaryotic cell, such as an antigen presenting cell, or a subset of antigen presenting cells, such as macrophage cells. A “target” is generally defined by the targeting element or property used to differentiate the target from non-targets. For example, a cancer cell can be targeted by a specific marker that recognizes molecules, such as surface receptors, that are specific to cancer cells. “Localization Signal” or “Localization Sequence” or “Recognition Sequence” or “Targeting Signal” or “Recognition Sequence” or “Recognition Tag” or “Recognition polynucleotide” are used interchangeably and refer to a signal that directs a molecule to a specific cell, tissue, organelle, or intracellular region. The signal can be polynucleotide, polypeptide, or carbohydrate moiety or can be an organic or inorganic compound sufficient to direct an attached molecule to a desired location.

The term “vector” refers to a delivery vehicle, such as a replicating RNA, a plasmid, phage, or cosmid, into which a DNA segment may be inserted so as to bring about the replication of the inserted segment. The vectors described herein can be expression vectors.

The term “expression vector” refers to a vector that includes one or more expression control sequences.

The term “heterologous” or “exogenous” refers to elements occurring where they are not normally found. For example, a promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter. The term “heterologous” thus can also encompass “non-native” or “non-self” elements.

As used herein, the term “nucleic acid” refers to any natural or synthetic linear and sequential arrays of nucleotides and nucleosides, for example cDNA, genomic DNA, mRNA, tRNA, oligonucleotides, oligonucleosides and derivatives thereof. Such nucleic acids may be collectively referred to herein as “constructs,” or “plasmids. Representative examples of the nucleic acids include bacterial plasmid vectors including expression, cloning, cosmid and transformation vectors such as, but not limited to, viral vectors, vectors derived from bacteriophage nucleic acid, and synthetic oligonucleotides like chemically synthesized DNA or RNA. The term “nucleic acid” further includes modified or derivatized nucleotides and nucleosides such as, but not limited to, halogenated nucleotides such as, but not only, 5-bromouracil, and derivatized nucleotides such as biotin-labeled nucleotides.

As used herein, the term “gene” or “genes” refers to isolated or modified nucleic acid sequences, including both RNA and DNA, that encode genetic information for the synthesis of a whole RNA, a whole protein, or any portion of such whole RNA or whole protein. Genes that are not naturally part of a particular organism's genome are referred to as “foreign genes”, “heterologous genes” or “exogenous genes” and genes that are naturally a part of a particular organism's genome are referred to as “endogenous genes”. The term “gene” as used herein with reference to genomic DNA includes intervening, non-coding regions as well as regulatory regions and can include 5′ and 3′ ends.

The term “expressed” or “expression” as used herein refers to the transcription from DNA to an RNA nucleic acid molecule at least complementary in part to a region of one of the two nucleic acid strands of the gene. The term “expressed” or “expression” as used herein also refers to the translation from said RNA nucleic acid molecule to give a protein or polypeptide or a portion thereof.

The term “iRNA” refers to a ribonucleic acid (RNA) molecule which can bind by complementary base pairing to a target messenger RNA transcripts (mRNAs), usually causing translational repression or target degradation which results in gene silencing reduced gene expression. Exemplary iRNA molecules include, but are not limited to, short interfering RNA (siRNA) and micro RNA (miRNA).

The terms “target gene” and “target sequence” are used interchangeably and refer to a sequence that can hybridize with an iRNA and induce gene silencing.

The term “selectable marker gene” as used herein refers to an expressed gene that allows for the selection of a population of cells containing the selectable marker gene from a population of cells not having the expressed selectable marker gene. For example, the “selectable marker gene” may be an “antibiotic resistance gene” that can confer tolerance to a specific antibiotic by a microorganism that was previously adversely affected by the drug. Such resistance may result from a mutation or the acquisition of resistance due to plasmids containing the resistance gene transfoiming the microorganism.

As used herein, “complexed” means associated by way of an electrostatic interaction.

The term “polypeptide” includes proteins and fragments thereof. Polypeptides are described herein as amino acid residue sequences. Those sequences are written left to right in the direction from the amino (N) to the carboxyl (C) terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “antibody” is used in the broadest sense unless clearly indicated otherwise. Therefore, an “antibody” can be naturally occurring or man-made, such as monoclonal antibodies produced by conventional hybridoma technology. Antibodies include monoclonal and polyclonal antibodies as well as fragments containing the antigen-binding domain and/or one or more complementarity determining regions of these antibodies. “Antibody” refers to any form of antibody or antigen binding fragment thereof and includes monoclonal antibodies (including full length monoclonal antibodies), polyclonal antibodies, multi-specific antibodies (e.g., bi-specific antibodies), and antibody fragments.

The term “antigen” as used herein refers to any substance (e.g., peptide, protein, nuclei acid, lipid, small molecule, such as a moiety expressed by or otherwise associated with a pathogen or cancerous or pre-cancerous cell) that serves as a target for the receptors of an adaptive immune response. The antigen may be a structural component of a pathogen, cancerous or pre-cancerous cell.

The term “pathogen” as used herein refers to an organism or other entity that causes a disease. For example, pathogens can be prions, viruses, prokaryotes such as bacteria, eukaryotes such as protozoa and fungi. A pathogen can be the source of an antigen to which an adaptive immune response can be generated.

As used herein, the term “eukaryote” or “eukaryotic” refers to organisms or cells or tissues derived therefrom belonging to the phylogenetic domain Eukarya such as animals (e.g., mammals, insects, reptiles, and birds), ciliates, plants (e.g., monocots, dicots, and algae), fungi, yeasts, flagellates, microsporidia, and protists.

As used herein, the term “prokaryote”, “prokaryotic cell” or “non-eukaryotic organism” refers to organisms including, but not limited to, organisms of the Eubacteria phylogenetic domain, such as Escherichia coli, Thermus thermophilus, and Bacillus stearothermophilus, or organisms of the Archaea phylogenetic domain such as, Methanocaldococcus jannaschii, Methanothermobacter thermautotrophicus, Halobacterium such as Haloferax volcanii and Halobacterium species NRC-1, Archaeoglobus fulgidus, Pyrococcus furiosus, Pyrococcus horikoshii, and Aeuropyrum pernix.

The term “biodegradable” as used herein means that the materials degrades or breaks down into its component subunits, or digestion, e.g., by a biochemical process, of the material into smaller (e.g., non-polymeric) subunits.

The term “polyplex” as used herein refers to polymeric micro- and/or nanoparticles or micelles having encapsulated therein, dispersed within, and/or associated with the surface of, one or more polynucleotides.

The terms “biocompatible” as used herein refers to one or more materials that are neither themselves toxic to the host (e.g., an animal or human), nor degrade (if the polymer degrades) at a rate that produces monomeric or oligomeric subunits or other byproducts at toxic concentrations in the host.

“Phagosomal/lysosomal escape” as used herein means the egress from within the endosomal or phagosomal compartment of a cell, such as an eukaryotic cell, to a non-endosomal or phagosomal space within the same cell, such as the cyctoplasm.

The term “biodegradable” as used herein means that the materials degrades or breaks down into its component subunits, or digestion, e.g., by a biochemical process, of the polymer into smaller, non-polymeric subunits.

### II. Compositions

It has been established that modified recombinant prokaryotic cells complexed with cationic polymers (hybrid vectors) can serve as vehicles for enhancing the targeted delivery of cargo, such as exogenous polypeptides and/or nucleic acids, to eukaryotic cells. The bacterial hybrid vectors can be designed and modified to deliver a broad range of intracellular cargo to eukaryotic cells, such as antigen presenting cells. Pharmaceutical compositions including the bacteria hybrid vectors in an amount effective to deliver one or more protein or nucleic acid cargos to a subject to achieve a desired clinical or biological effect in the subject are provided.

A. Hybrid Bacterial Cell Vectors

Hybrid vectors including cationic polymers complexed with prokaryotic cells containing one or more nucleic acid and/or protein for delivery to eukaryotic cells (hybrid bacterial vectors) typically include one or more plasmids engineered to express a heterologous sequence in an eukaryotic target cell.

Hybrid bactefial vectors including biodegradable cationic polymers as vehicles for the targeted delivery of nucleic acids, proteins and small molecules to antigen-presenting cells typically include one or more biodegradable cationic polymers associated with the exterior surface of the prokaryote cell. The prokaryote cell is preferably a bacterial cell such as Escherichia Spp.

Hybrid bacterial vectors can include a plasmid engineered to express a heterologous sequence in an eukaryotic cell, and at least one of (i) a gene encoding a lytic enzyme, such as one that encodes for listeriolysin O (“LLO”) and (ii) a gene that codes for an endolysin or a catalytic domain thereof. In some embodiments the bacteria hybrid vectors include a gene that encodes the LLO protein and a gene that encodes an endolysin, or a biologically active domain thereof.

Hybrid bacterial vectors can be useful for the enhanced delivery of genetic and protein material to antigen presenting cells. It may be that surface deposition of cationic polymers to the bacterial core results in a beneficial attenuation phenomenon that is driven by a mild disruption of the outer bacterial membrane, neutralization of excess charge of the polymer constituent, and reduced exposure of immunogenic molecules, such as LPS.

The hybrid bacterial vectors are designed to facilitate uptake by eukaryotic cells and include one or more recombinant proteins that facilitate egress of the nucleic acid and/or protein cargo from the lysosomal or endosomal pathway. Hybrid bacterial vectors optionally include one or more targeting motifs to enhance specificity and uptake by the target cell type. The hybrid bacterial vectors are non-toxic and typically have a size amenable for uptake by immune cells such as macrophages. An exemplary size for a single hybrid vector is in the range of 0.5 to 10 micrometers in the longest dimension, for example, hybrid vectors can have an average size from 1 to 5 μm, inclusive.

1. Cationic Polymers

Cationic polymers represent a common biomaterial vector. Within the cationic polymer category, various classes of polymers have are available that feature facile synthesis, innate gene packaging properties, low cytotoxicity, and toolsets to permit rapid tailoring for specific applications.

The cationic polymers can be biodegradable, and can be associated with the outer surface of the bacteria in an amount sufficient to result in a net positive surface charge of the bacteria/polymer vector. The net positive charge can enhance attraction and uptake by eukaryotic cells such as antigen presenting cells. Thus, in preferred embodiments surface addition of cationic polymers results in the permeation of bacteria without causing gross bactericidal effects.

Cationic polymers suitable for use in the described hybrid devices include synthetically-derived biodegradable cationic polymers. A non-limiting list of exemplary polymers includes poly(beta-amino esters) (“PBAE”) (Angew. Chem. Intl Ed 42 (27): 3153-3158; JACS 122 (44): 10761-10768), including those with pyridyldithio groups in the polymer side chains; aliphatic polyesters such as cationic polylactide (“PLA” or “CPLA”) (Adv. Healthc. Mater. 1, 751-761; Mol. Pharm. 10, 1138-1145;); poly(trans-4-hydroxy-L-proline ester) (“PHP”) (JACS 121 (24): 5633-5639); poly[α-(4-aminobutyl)-L-glycolic acid] (“PAGA”) (JACS 122 (4): 6524-6525); polyphosphoesters such as poly(2-amioethyl propylene phosphate) (“PPE-EA”) (see JACS 123 (38): 9480-9481); polyphosphazenes (“PPAs”) (J Controlled Release 89 (3): 483-497); poly(L-lysine) containing disulfide linkages in the polymer side chains such as poly[Lys-(AEDTP)] (Bioconj Chem 13 (1): 76-82); disulfide-containing polymers such as poly(amido amine) (SS-PAA) (co)polymers; polyethylenimine (PEI); DTSP or DTBP crosslinked PEI (Bioconj Chem 12 (6): 989-994); PEGylated PEI crosslinked with DTSP (J Contr Rel 124(1-2): 69-80); Crosslinked PEI with DSP (J Contr Rel 118 (3): 370-380); Linear SS-PEI (Bioconj Chem 18 (1):13-18); DTSP-Crosslinked linear PEI (2-4 kDa) (PNAS 92 (16):7297-7301); PEI-SS(x) from thiolated 800-kDa (Bioconj Chem 19 (2): 4099-506); branched poly(ethylenimine sulfide) (b-PEIS) (Biomat 31 (5): 988-997); Poly(2-N,N-dimethylaminoethylmethacrylate) (PDMAEMA); Poly(amino-co-ester) (PAE) (see Samal, et al., Chem Soc Rev, DOI: 10.1039/c2cs35094g (2012)) as well as derivatives, variants and modified forms thereof.

Example preparation schemes for each of these biodegradable cationic polymers are known in the art. Details of the manufacturing process are also provided herein in Example 1.

Bacterial hybrid vectors can be formed using a single cationic polymer species, or a mixture of multiple different cationic polymer species.

Biodegradable cationic polymers for use in the described hybrid vectors (“CPs”) can be linear, branched or dendritic structures. Typically, ye

CPs have a charge density that is consistent with forming electrostatic interactions with a prokaryotic cell. For example, the charge density of the biodegradable CP can be between −50 and −30 mV. Charge density within the polymer backbone can be precisely controlled by techniques known in the art. Specifically, successful synthesis of controlled charge density additions has been reported using atom transfer radical polymerization (ATRP) (Plos One 5, (1):e8668.; Mol Ther., 17(1):65-72), reversible addition-fragmentation chain-transfer polymerization (RAFT) (Biomaterials 33(13):3594-3603; Mol Ther., 15(7):1306-1312), ring-opening polymerization (J Immunol., 178(10):6259-6267), click-chemistry (Cur Opin in Mol Ther., 7(2):157-163; Clin Cancer Res., 10(6):1920-1927), and other various chemical procedures such as Michael additions. These strategies are reproducible and can facilitate synthesis of well-defined charge density polymers.

Typically, the charge density of the biodegradable cationic polymer is between −50 and −30 mV, inclusive.

In certain embodiments the molecular weight of the biodegradable CP used in the hybrid vectors is between approximately 500 Da and 20,000 Da, inclusive. For example, the molecular weight of one or more biodegradable cationic polymers can be from approximately 1,000 Da to 10,000 Da, inclusive. Preferably the molecular weight of one or more biodegradable cationic polymers is approximately 5000-6000 Da.

In some embodiments, the biodegradable cationic polymers is a PBAE. A preferred biodegradable cationic polymer is poly(beta-amino ester). Poly(beta-amino ester) (PBAE) can be synthesized including a variety of different diacrylates with amines, for example, according to scheme I, illustrated below:

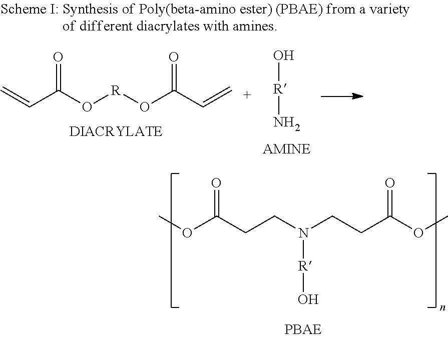

A non-limiting list of diacrylates and amines that can be combined according to this scheme to produce biodegradable polymers is provided in Table 1.

In certain embodiments, monomers containing two or more alcohol groups, or two or more amine groups are most beneficial for gene delivery.

TABLE 1
Exemplary Diacrylates and Amines

| Diacrylates | | Amines | |
|---|---|---|---|
| A | 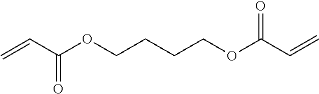 | 1 | 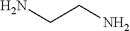 |
| B | 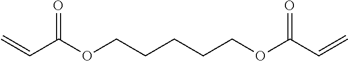 | 2 | 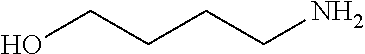 |
| C | 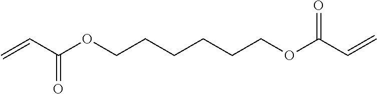 [SMILES: C=CC(=O)OCCCCCCOC(=O)C=C] | 3 | 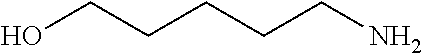 |
| D | 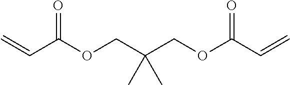 | 4 | 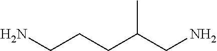 |
| E | 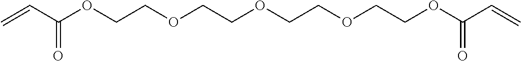 [SMILES: C=CC(=O)OCCOCCOCCOCCOC(=O)C=C] | 5 | 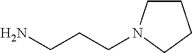 |
| F | 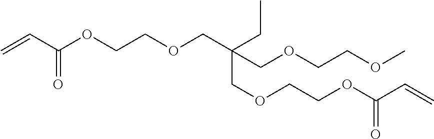 [SMILES: C=CC(=O)OCCOCC(CC)(COCCOC)COCCOC(=O)C=C] | 6 | 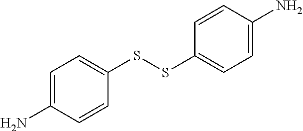 [SMILES: Nc1ccc(SSc2ccc(N)cc2)cc1] |
| G | 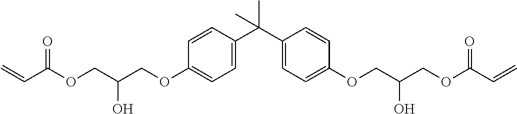 [SMILES: C=CC(=O)OCC(O)COc1ccc(C(C)(C)c2ccc(OCC(O)COC(=O)C=C)cc2)cc1] | 7 | 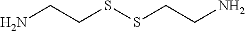 |
| | | 8 | 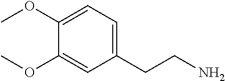 |
| | | 9 | 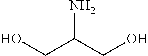 |
| | | 10 | 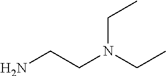 |
| | | 11 | 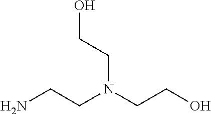 |
| | | 12 | 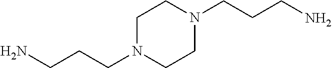 [SMILES: NCCCN1CCN(CCCN)CC1] |
| | | 13 | 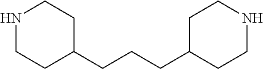 [SMILES: C(CC1CCNCC1)CC1CCNCC1] |

In some embodiments the cationic polymer is a PBAE synthesized by conjugate addition of Neopentyl glycol diacrylate to 2-Amino-1,3-propanediol. A preferred cationic polymer is Acrylate-terminated poly(neopentyl glycol diacrylate-co-2-amino-1,3-propanediol)) (also termed D9) having a molecular structure according to Formula I.

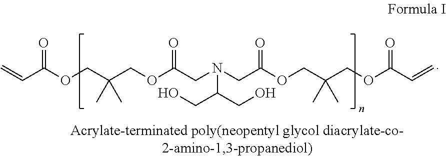

i. Modifications of Cationic Polymers

The cationic polymers can be modified by the addition of one or more adducts, including but not limited to any biocompatible, non-toxic polymer or copolymer, for example, a poly(alkylene glycol), a polysaccharide, poly(vinyl alcohol), polypyrrolidone, a polyoxyethylene block copolymer (PLURONIC®) or a copolymers thereof. In some embodiments, one or more cationic polymers are modified by addition of polyethylene glycol (PEG). In other embodiments, one or more cationic polymers are modified by addition of carbohydrates such as mannose. In further embodiments, one or more cationic polymers are modified by addition of polypeptides or other small molecules. Typically, modifications impart distinct structural and functional properties to the polymers. Therefore, modified cationic polymers can be used to impart one or more distinct functional or structural properties to the hybrid bacterial vector, as compared to the same hybrid vector in the absence of the modification. Exemplary functional or structural properties include variation of the hydrodynamic volume, hydrophobicity, antigenicity, receptor-binding specificity and serum half-life of the hybrid vector.

Bacterial hybrid vectors can be formed using a single modified cationic polymer species, or a mixture of multiple different cationic polymer species modified with the sane or different adducts. For example, bacterial hybrid vectors can be formed using one or more cationic polymers including mannose adducts, or one or more cationic polymers including poly(ethylene glycol), or mixtures of the same or different cationic polymers modified with mannose and cationic polymers modified with poly(ethylene glycol).

a. Hydrophilic Polymers

The cationic polymers can be modified by addition of a hydrophilic polymer or copolymer. Preferred polymers are biocompatible (i.e., do not induce a significant inflammatory or immune response) and non-toxic.

Examples of suitable hydrophilic polymers include, but are not limited to, poly(alkylene glycols) such as polyethylene glycol (PEG), poly(propylene glycol) (PPG), and copolymers of ethylene glycol and propylene glycol, poly(oxyethylated polyol), poly(olefinic alcohol), polyvinylpyrrolidone), poly(hydroxyalkylmethacrylamide), poly(hydroxyalkylmethacrylate), poly(saccharides), poly(amino acids), poly(hydroxy acids), polyvinyl alcohol), and copolymers, terpolymers, and mixtures thereof.

In preferred embodiments, the one or more hydrophilic polymer segments contain a poly(alkylene glycol) chain. The poly(alkylene glycol) chains may contain between 1 and 500 repeat units, more preferably between 40 and 500 repeat units. Suitable poly(alkylene glycols) include polyethylene glycol, polypropylene 1,2-glycol, poly(propylene oxide), polypropylene 1,3-glycol, and copolymers thereof.

In some embodiments, the one or more hydrophilic polymer segments are copolymers containing one or more blocks of polyethylene oxide (PEO) along with one or more blocks composed of other biocompatible polymers (for example, poly(lactide), poly(glycolide), poly(lactide-co-glycolide), or polycaprolactone). The one or more hydrophilic polymer segments can be copolymers containing one or more blocks of PEO along with one or more blocks containing polypropylene oxide (PPO). Specific examples include triblock copolymers of PEO—PPO-PEO, such as POLOXAMERS™ and PLURONICS™.

1. Poly(Ethylene Glycol)

In some embodiments, cationic polymers are modified by the addition of polyethylene glycol (PEG). PEG is one of the most commonly used shielding agents. Well-defined PEGylated cationic polylactides (PEG-b-CPLA) have varying charge densities. The addition of PEG to the described cationic polymers (PEGylation) can affect structural features and charge density of the prokaryotic cells modified with the polymers. Therefore, in certain embodiments, one or more properties of the hybrid bacterial vectors can be altered by pegylation of the cationic polymers. Exemplary properties that can be modified include uptake of the vector by eukaryotic eclls, the speed and efficacy of gene delivery efficacy, immunogenicity and cytotoxicity of the vector. In certain embodiments, addition of PEG to a polymer results in charge neutralization of the hybrid bacterial vector.

Typically, the PEG modification includes a short-chain oligo-ethylene glycol. Exemplary oligoi-ethylene glycols include di-ethylene glycol, tri-ethylene glycol, tetra-ethylene glycol, penta-ethylene glycol, hexa-ethylene glycol, etc.

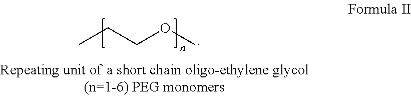

b. Carbohydrates

In some embodiments the cationic polymers can be modified by addition of one or more carbohydrate moieties. Carbohydrate moieties can be recognized by specific receptor molecules at the surface of eukaryotic cells, such as lectins.

Preferably, the carbohydrate is a mannose moiety (i.e., mannosylation). Mannose moieties can facilitate targeting of hybrid bacterial vectors to cells bearing surface receptors that recognize mannose, such as CD206. The CD206 marker, also known as the mannose receptor, is the product of the MRC1 gene, expressed at the surface of antigen presenting cells such as macrophages and other dendritic cells (Ezekowitz, et al., J Exp Med. 1; 172(6):1785-94 (1990)). The mannose receptor is a pattern recognition receptor that binds to terminal mannose at the surface of pathogens. Following binding to the mannose receptor, the mannose-bound pathogen is then internalized and transported to the lysosomes for degradation via the phagocytic pathway (Kerrigan and Brown, Immunobiology, 214(7): 562-575 (2009)). Therefore, the polymer component of the bacterial hybrid vector can be modified by the terminal conjugation of mannose to improve specificity of uptake by antigen presenting cell (APC) through CD206 stimulation.

In certain embodiments, terminal conjugation of mannose to the polymer component can improve the specificity of antigen presenting cell (APC) uptake through CD206 stimulation. The molecular weight and relative mannose content of the cationic polymer can be optimized for uptake by one or more cell types.

The mannosylation of cationic polymers can impart distinct characteristics to the polymers as compared to the same polymers in the absence of mannose. For example, in certain embodiments mannosylation results in an enhanced coating efficiency, or greater surface coverage as compared to the coating efficiency or surface coverage by the same polymers in the absence of mannose. In certain embodiments, the addition of mannosylated cationic polymers increases the polymer-mediated membrane disruption of the prokaryotic cell to which the polymers are associated. The increase in polymer-mediated membrane disruption can be positively correlated with the extent of mannosylation (i.e., positively correlated with increasing mass:mass ratio of the mannose to the polymer). In certain embodiments, the addition of mannose results in changes in the zeta potential of the cationic polymer. For example, increasing amounts of mannose can give rise to an increasing zeta potential.

In certain embodiments, mannosylation of the cationic polymer can effects the hydrophobicity of the bacterial hybrid vector. For example, hydrophobicity can be positively correlated with increased mannosylation. Mannosylation can also reduce coalescence and aggregation of the bacterial hybrid vectors

Typically, mannosylation improves the uptake and delivery of cargo by a target cell, such as a cell bearing the CD206 receptor. For example, a mannosylated bacterial hybrid vector can enhance gene delivery to an antigen presenting cell relative to gene delivery to the same cell or cell type by an equivalent bacterial hybrid vector in the absence of mannosylation. Typically, mannosylation improves the toxicity profile of the bacterial hybrid vector. For example, the addition of mannose can reduce cytotoxicity, or has minimal effect upon cytotoxicity relative to the equivalent bacterial hybrid vector in the absence of mannosylation. It may be that reduced cytotoxicity and enhanced delivery of nucleic acids and polypeptides to eukaryotic cells occurs as a result of enhanced polymer degradation and/or charge-mediated bacterial attenuation. The effects of mannosylation can be independent of the chemical nature of the polymer.

The addition of mannose to cationic polymers can occur by any means known to those skilled in the art. For example, the addition of mannose can occur via chemical means of via free addition. Typically, mannose is converted to Ally-alpha-D-mannopyranoside (ADM) by heating in the presence of ally-alcohol, according to scheme II, below:

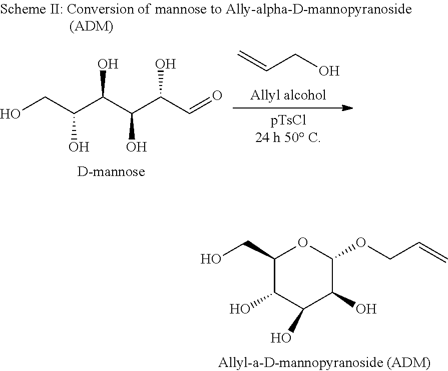

A preferred mannosylated polymer is the mannosylated derivative of Acrylate-terminated poly(neopentyl glycol diacrylate-co-2-amino-1,3-propanediol)) (also termed D9-Man), having the molecular structure of Formula III.

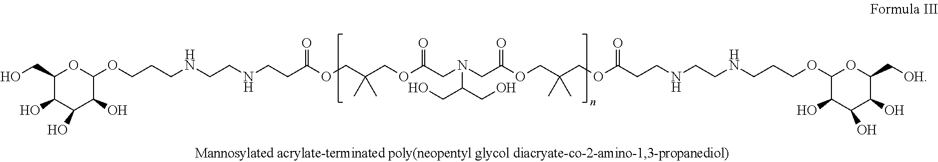

c. Additional Functional Groups

The hybrid bacterial vectors can include one or more additional functional groups. One or more additional functional groups can be added to the biodegradable CPs at one or both ends, using any protocols known in the art, for example, end-capping. For example, the cationic polymers can be coated with surface charge altering materials, polypeptides that increase stability and half-life of the hybrid vectors in systemic circulation, and/or a targeting moiety that increases targeting of the particles to a cell type or cell state of interest.

Exemplary functional groups include targeting elements, immune-modulatory elements, chemical groups, biological macromolecules, and combinations thereof.

1. Immune-Modulatory Elements

Cationic polymers for use in formation of hybrid bacterial vectors can include immune-modulatory factors. Exemplary immune-modulatory factors include cytokines, xanthines, interleukins, interferons, oligodeoxynucleotides, glucans, growth factors (e.g., TNF, CSF, GM-CSF and G-CSF), hormones such as estrogens (diethylstilbestrol, estradiol), androgens (testosterone, HALOTESTIN® (fluoxymesterone)), progestins (MEGACE® (megestrol acetate), PROVERA® (medroxyprogesterone acetate)), corticosteroids (prednisone, dexamethasone, hydrocortisone), CRM197 (diphtheria toxin), outer membrane protein complex from Neisseria meningitides, as well as viral hemagglutinin and neuraminidase.

In certain embodiments, the cationic polymers include immuno-stimulatory factors. Exemplary immuno-stimulatory factors include, but are not limited to, TLR ligands, C-Type Lectin Receptor ligands, NOD-Like Receptor ligands, RLR ligands, and RAGE ligands. TLR ligands can include lipopolysaccharide (LPS) and derivatives thereof, as well as lipid A and derivatives there of including, but not limited to, monophosphoryl lipid A (MPL), glycopyranosyl lipid A, PET-lipid A, and 3-O-desacyl-4′-monophosphoryl lipid A.

2. Targeting Elements

The hybrid bacterial vectors can include targeting moieties that enhance uptake by eukaryote cells. Exemplary targeting elements include proteins, peptides, nucleic acids, lipids, saccharides, or polysaccharides that bind to one or more targets associated with an organ, tissue, cell, or extracellular matrix, or specific type of tumor or infected cell. The degree of specificity with which the delivery vehicles are targeted can be modulated through the selection of a targeting molecule with the appropriate affinity and specificity. For example, antibodies, or antigen-binding fragments thereof are very specific.

Typically, the targeting moieties exploit the surface-markers specific to a biologically functional class of cells, such as antigen presenting cells. Dendritic cells express a number of cell surface receptors that can mediate endocytosis. Targeting exogenous antigens to internalizing surface molecules on systemically-distributed antigen presenting cells facilitates uptake of the particle and can overcomes a major rate-limiting step in the delivery of nucleic acids and proteins to these cells. Dendritic cell targeting molecules include ligands which bind to a cell surface receptor on dendritic cells. One such receptor, the lectin DEC-205, has been used in vitro and in mice to boost both humoral (antibody-based) and cellular (CD8 T cell) responses by 2-4 orders of magnitude (Hawiger, et al., J. Exp. Med., 194(6):769-79 (2001); Bonifaz, et al., J. Exp. Med., 196(12):1627-38 (2002); Bonifaz, et al., J. Exp. Med., 199(6):815-24 (2004)). Other receptors on dendritic cells that can be targeted include Fc gamma RIIB; DCIR; Dectin-1; CLEC9A; Langerin; CD11c; CD163; FC gamma RIIB; DC-SIGN, 33D1, SIGLEC-H, TLRs, heat shock protein receptors and scavenger receptors.

In some embodiments the targeting domain can enhance targeting of hybrid bacterial vectors to cancer cells.

Antibodies

Antibodies that function by binding directly to one or more epitopes, other ligands or accessory molecules at the surface of eukaryote cells, are described. Typically, the antibody or antigen binding fragment thereof has affinity for a receptor at the surface of a specific cell type, such as a receptor expressed at the surface of macrophage cells.

Any specific antibody can be used in the methods and compositions provided herein. For example, antibodies can include an antigen binding site that binds to an epitope on the target cell. Binding of an antibody to a “target” cell can enhance or induce uptake of hybrid bacterial vectors by the target cell protein via one or more distinct mechanisms.

In some embodiments, the antibody or antigen binding fragment binds specifically to an epitope. The epitope can be a linear epitope. The epitope can be specific to one cell type or can be expressed by multiple different cell types. In other embodiments, the antibody or antigen binding fragment thereof can bind a conformational epitope that includes a 3-D surface feature, shape, or tertiary structure at the surface of the target cell.

In some embodiments, the antibody or antigen binding fragment that binds specifically to an epitope on the target cell can only bind if the protein epitope is not bound by a ligand or small molecule.

Various types of antibodies and antibody fragments can be used in the described compositions and methods, including whole immunoglobulin of any class, fragments thereof, and synthetic proteins containing at least the antigen binding variable domain of an antibody. The antibody can be an IgG antibody, such as IgG₁, IgG₂, IgG₃, or IgG₄. An antibody can be in the form of an antigen binding fragment including a Fab fragment, F(ab′)2 fragment, a single chain variable region, and the like. Antibodies can be polyclonal or monoclonal (mAb). Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity (U.S. Pat. No. 4,816,567; and Morrison, et al., Proc. Natl. Acad. Sci. USA, 81: 6851-6855 (1984)). The described antibodies can also be modified by recombinant means, for example by deletions, additions or substitutions of amino acids, to increase efficacy of the antibody in mediating the desired function. Substitutions can be conservative substitutions. For example, at least one amino acid in the constant region of the antibody can be replaced with a different residue (see, e.g., U.S. Pat. No. 5,624,821; U.S. Pat. No. 6,194,551; WO 9958572; and Angal, et al., Mol. Immunol. 30:105-08 (1993)). In some cases changes are made to reduce undesired activities, e.g., complement-dependent cytotoxicity. The antibody can be a bi-specific antibody having binding specificities for at least two different antigenic epitopes. In one embodiment, the epitopes are from the same antigen. In another embodiment, the epitopes are from two different antigens. Bi-specific antibodies can include bi-specific antibody fragments (see, e.g., Hollinger, et al., Proc. Natl. Acad. Sci. U.S.A., 90:6444-48 (1993); Gruber, et al., J. Immunol., 152:5368 (1994)).

Antibodies that target the hybrid bacterial vectors to a specific epitope can be generated by any means known in the art. Exemplary descriptions means for antibody generation and production include Delves, Antibody Production: Essential Techniques (Wiley, 1997); Shephard, et al., Monoclonal Antibodies (Oxford University Press, 2000); Goding, Monoclonal Antibodies: Principles And Practice (Academic Press, 1993); and Current Protocols In Immunology (John Wiley & Sons, most recent edition). Fragments of intact Ig molecules can be generated using methods well known in the art, including enzymatic digestion and recombinant means.

2. Prokaryote Cells

Hybrid vectors can be formed from any suitable prokaryotic cell. The cells can be a live cell, an attenuated cell or an inactivated cell. Typically, the prokaryotic cell is a bacterial cell. The inactivated cells for use with described hybrid vectors can be inactivated by any suitable means known in the art, such as through UV irradiation, heat, chemical treatment, lyophilization, etc. The bacterial cells of the described hybrid vectors typically have a net-negative surface charge, such that complexing between the cell and the cationic polymer molecules is driven by electrostatic interactions between the positively-charged polymers and the negatively-charged outer surface of the cell.

a. Escherichia coli.

An exemplary prokaryotic cell is a bacterium belonging to the species Escherichia coli.

Escherichia coli is a rod-shaped, facultative anaerobic Gram-negative bacterium that measures approximately 0.5 μm in diameter by 2 μm in length and has a rapid rate of growth (Parsa S, Pfeifer B., Mol. Pharm. 4(1):4-17(2007)). Escherichia coli cells natively promote phagocytic uptake by APCs, and upon internalization.

Thus, in some embodiments hybrid vectors include an Escherichia coli component that is a live, un-attenuated bacterium. In other embodiments, the hybrid vectors include an Escherichia coli component that is a live, attenuated bacterium. In other embodiments, the Escherichia coli component of the hybrid vector is comprised of an inactivated (i.e., killed/dead) bacterium.

In certain embodiments, the hybrid vectors include an Escherichia coli component that is a live, non-pathogenic strain of Escherichia coli. Non-pathogenic strains of Escherichia coli exhibit minimal toxicity and immunogenicity. Escherichia coli strains that can be used in the formation of bacterial hybrid vectors include all strains and sub-strains known in the art, including but not-limited to Escherichia coli RR1; Escherichia coli LE392; Escherichia coli B; Escherichia coli 1776 (ATCC No. 31537); Escherichia coli W3110 (F-, lambda-, prototrophic, ATCC No. 273325); Escherichia coli strain YWT7-hly; Escherichia coli K; Escherichia coli BL21-DE3, Nissle 1917, K-12 (including Clifton wild type; DH5αE; Dam dcm strain); Escherichia coli REL606; Escherichia coli strain C; Escherichia coli strain W; as well as genetically modified variants thereof. The complete genome of a representative Escherichia coli K-12 sub-strain MG1655 is available (Freddolino, et al. J Bacteriol 2012).

The Escherichia coli can be inherently non-pathogenic or engineered to be non-pathogenic through methods such as expression of the Lysis E gene natively found in phage pX174. In a preferred embodiment, the E. coli strain is an inherently non-pathogenic strain, such as Escherichia coli include B and K derivatives. In a particular embodiment, the Escherichia coli strain is the S1 (YWT7-hly) strain.

3. Nucleic Acid Expression Vectors

The prokaryotic cells used in the formation of the hybrid vectors can include one or more plasmids engineered to express a heterologous sequence in a target cell, such as an eukaryotic cell. Typically, the expression vector includes a promoter, a heterologous nucleic acid sequence operably linked to the promoter, an eukaryotic transcription terminator operably linked to the heterologous nucleic acid sequence, and an origin of replication.

In general, plasmid vectors containing replicon and control sequences that are derived from species compatible with the host cell are used in connection with these hosts. The vector ordinarily carries a replication site, as well as marking sequences that are capable of providing phenotypic selection in transformed cells. For example, Escherichia coli is often transformed using pBR322, a plasmid derived from an Escherichia coli strain. Plasmid pBR322 contains genes for ampicillin and tetracycline resistance and thus provides easy means for identifying transformed cells. The pBR322 plasmid, or other microbial plasmid or phage must also contain, or be modified to contain, promoters that can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector that can be used to transform host cells, such as Escherichia coli LE392.

Different host cells have characteristic and specific mechanisms for the post-translational processing and modification of proteins. Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences.

In cases where an adenovirus is used as an expression vector, the coding sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing proteins in infected hosts.

In certain embodiments, the plasmid may also include a selectable marker.

a. Promoter

The promoter within the plasmid can be any promoter that is active in vivo. Exemplary promoters are known in that art, including constitutive promoters and regulated promoters. Promoters can be of eukaryotic, prokaryotic or viral origin. An exemplary regulated promoter is the IPTG-inducible promoter. For example, prokaryotic promoters can be used to express polypeptides in prokaryotic cells prior to delivery to an eukaryotic cell.

The promoter can be specific to eukaryotic cells or to prokaryotic cells, or can lack specificity. In certain embodiments, the promoter is specific to one or more particular tissue or cell type. In other embodiments, the promoter is active in many or all tissue and cell types.

The promoters may be derived from the genome of mammalian cells (e.g., metallothionein promoter) or from mammalian viruses (e.g., the adenovirus late promoter; the vaccinia virus 7.5K promoter). Further, it is also possible, and may be desirable, to utilize promoter or control sequences normally associated with the desired gene sequence, provided such control sequences are compatible with the host cell systems. Commonly used promoters are derived from polyoma, Adenovirus 2, cytomegalovirus and Simian Virus 40 (SV40). The early and late promoters of SV40 virus are useful because both are obtained easily from the virus as a fragment which also contains the SV40 viral origin of replication. Smaller or larger SV40 fragments may also be used, provided there is included the approximately 250 bp sequence extending from the HinDIII site toward the BglI site located in the viral origin of replication.

A non-limiting list of exemplary eukaryotic promoters includes those from phosphosglycerate kinase, chicken beta-actin, human elongation factor-alpha (EF-1α), human H1 and U6 promoters. A non-limiting list of exemplary viral promoters includes those from cytomegalovirus (CMV), Rous sarcoma virus (RSV), simian vacuolating virus 40 (SV40). A non-limiting list of exemplary prokaryote promoters, includes bacterial promoters, such as Tet (tetracycline) or T7 promoters. In certain embodiments bacterial promoters can be used to express the antigen in a bacterial cell prior to delivery to the mammalian cell.

Hybrid viral/eukaryotic promoters can also be included in the expression plasmids. An exemplary hybrid viral/eukaryotic promoter is chicken-β actin with CMV early enhancer (CAGG) promoter.

Specific initiation signals may also be required for efficient translation of exogenous nucleic acid coding sequences. These signals include the ATG initiation codon and adjacent sequences. Exogenous translational control signals, including the ATG initiation codon, may additionally need to be provided. One of ordinary skill in the art would readily be capable of determining this need and providing the necessary signals. It is well known that the initiation codon must be in-frame (or in-phase) with the reading frame of the desired coding sequence to ensure translation of the entire insert. Exogenous translational control signals and initiation codons can be of a variety of origins, both natural and synthetic. The efficiency of expression may be enhanced by the inclusion of appropriate transcription enhancer elements or transcription terminators. In eukaryotic expression, an appropriate polyadenylation site (e.g., 5′-AATAAA-3′) can also be incorporated into the transcriptional unit if not contained within the original cloned segment. Typically, the poly A addition site is placed about 30 to 2000 nucleotides downstream of the termination site of the protein at a position prior to transcription termination.

b. Heterologous Nucleic Acid Sequence

The heterologous nucleic acid sequence can be any nucleic acid sequence that that encodes genetic information for the synthesis of a portion of or a whole RNA, or a portion of or a whole protein, for the purpose of modulating gene expression, and/or production of one or more gene products.

In certain embodiments the heterologous nucleic acid sequence encodes an element that initiates and/or moderates a biological response in the host cell. For example, the heterologous protein can inhibit expression of one or more genes.

In some embodiments, the heterologous nucleic acid encodes an antigen.

A non-limiting list of exemplary list of heterologous nucleic acid sequences include genes encoding antigens, ribozymes, enzymes, peptides, structural proteins, structural RNA, shRNA, siRNA, miRNA, transcription factors, signaling molecules and fragments or variants thereof. In an embodiment, the heterologous sequence encodes an antigen.

The heterologous sequence may optionally contain a nucleic acid sequence (i.e., a targeting sequence) that enables targeting to a specific location (e.g. organelle within the cell). In some embodiments, the heterologous sequence encodes an antigen.

i. Antigens

In some embodiments the exogenous nucleic acid sequence encodes a vaccine antigen. An antigen can include any protein or peptide that is foreign to the subject organism.

Preferred antigens can be presented at the surface of antigen presenting cells (APC) of a subject for surveillance by immune effector cells, such as leucocytes expressing the CD4 receptor (CD4 T cells) and Natural Killer (NK) cells. Typically, the antigen is of viral, bacterial, protozoan, fungal, or animal origin. In some embodiments the antigen is a cancer antigen. Cancer antigens can be antigens expressed only on tumor cells and/or required for tumor cell survival

Certain antigens are recognized by those skilled in the art as immuno-stimulatory (i.e., stimulate effective immune recognition) and provide effective immunity to the organism or molecule from which they derive.

Antigens can be peptides, proteins, polysaccharides, saccharides, lipids, nucleic acids, or combinations thereof. The antigen can be derived from a virus, bacterium, parasite, plant, protozoan, fungus, tissue or transformed cell such as a cancer or leukemic cell and can be a whole cell or immunogenic component thereof, e.g., cell wall components or molecular components thereof. Suitable antigens are known in the art and are available from commercial government and scientific sources. The antigens may be purified or partially purified polypeptides derived from tumors or viral or bacterial sources. The antigens can be recombinant polypeptides produced by expressing DNA encoding the polypeptide antigen in a heterologous expression system. The antigens can be DNA encoding all or part of an antigenic protein. Antigens may be provided as single antigens or may be provided in combination. Antigens may also be provided as complex mixtures of polypeptides or nucleic acids.

Viral Antigens

A viral antigen can be isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3. Viral antigens may be derived from a particular strain such as a papilloma virus, a herpes virus, i.e. herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever,and lymphocytic choriomeningitis.

Bacterial Antigens

Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

In some embodiments the antigen is a polypeptide or protein associated with diseases of poultry, such as Infectious Bursal Disease (IBD) of chickens.

Parasite Antigens

Parasite antigens can be obtained from parasites such as, but not limited to, an antigen derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

Allergens and Environmental Antigens

The antigen can be an allergen or environmental antigen, such as, but not limited to, an antigen derived from naturally occurring allergens such as pollen allergens (tree-, herb, weed-, and grass pollen allergens), insect allergens (inhalant, saliva and venom allergens), animal hair and dandruff allergens, and food allergens. Important pollen allergens from trees, grasses and herbs originate from the taxonomic orders of Fagales, Oleales, Pinales and platanaceae including i.a. birch (Betula), alder (Alnus), hazel (Corylus), hornbeam (Carpinus) and olive (Olea), cedar (Cryptomeriaand Juniperus), Plane tree (Platanus), the order of Poales including i.e. grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other allergen antigens that may be used include allergens from house dust mites of the genus Dermatophagoides and Euroglyphus, storage mite e.g. Lepidoglyphys, Glycyphagus and Tyrophagus, those from cockroaches, midges and fleas e.g. Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

Tumor Antigens

The antigen can be a tumor antigen, including a tumor-associated or tumor-specific antigen, such as, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARa fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, b-Catenin, CDK4, Mum-1, p16, TAGE, PSMA, PSCA, CT7, telomerase, 43-9F, 5T4, 791Tgp72, a-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS.

ii. Functional Nucleic Acids

In certain embodiments the heterologous nucleic acid sequence is a functional nucleic acid. Functional nucleic acids that inhibit the transcription, translation or function of a target gene are described.

Functional nucleic acids are nucleic acid molecules that have a specific function, such as binding a target molecule or catalyzing a specific reaction. As discussed in more detail below, functional nucleic acid molecules can be divided into the following non-limiting categories: antisense molecules, siRNA, miRNA, aptamers, ribozymes, triplex forming molecules, RNAi, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the target polypeptide itself. Functional nucleic acids are often designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place. Therefore the compositions can include one or more functional nucleic acids designed to reduce expression or function of a target protein.

Methods of making and using vectors for in vivo expression of the described functional nucleic acids such as antisense oligonucleotides, siRNA, shRNA, miRNA, EGSs, ribozymes, and aptamers are known in the art.

Antisense Molecules

The functional nucleic acids can be antisense molecules. Antisense molecules are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively the antisense molecule is designed to interrupt a processing function that normally would take place on the target molecule, such as transcription or replication. Antisense molecules can be designed based on the sequence of the target molecule. There are numerous methods for optimization of antisense efficiency by finding the most accessible regions of the target molecule. Exemplary methods include in vitro selection experiments and DNA modification studies using DMS and DEPC. It is preferred that antisense molecules bind the target molecule with a dissociation constant (Kd) less than or equal to 10-6, 10-8, 10-10, or 10-12.

Aptamers

The functional nucleic acids can be aptamers. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with Kd's from the target molecule of less than 10-12 M. It is preferred that the aptamers bind the target molecule with a Kd less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule. It is preferred that the aptamer have a Kd with the target molecule at least 10, 100, 1000, 10,000, or 100,000 fold lower than the Kd with a background binding molecule. It is preferred when doing the comparison for a molecule such as a polypeptide, that the background molecule be a different polypeptide.

Ribozymes

The functional nucleic acids can be ribozymes. Ribozymes are nucleic acid molecules that are capable of catalyzing a chemical reaction, either intra-molecularly or inter-molecularly. It is preferred that the ribozymes catalyze intermolecular reactions. Different types of ribozymes that catalyze nuclease or nucleic acid polymerase-type reactions which are based on ribozymes found in natural systems, such as hammerhead ribozymes are described. Ribozymes that are not found in natural systems, but which have been engineered to catalyze specific reactions de novo are also described. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for targeting specific cleavage of nucleic acids because recognition of the target substrate is based on the target substrates sequence.

Triplex Forming Oligonucleotides

The functional nucleic acids can be triplex forming oligonucleotide molecules. Triplex forming functional nucleic acid molecules are molecules that can interact with either double-stranded or single-stranded nucleic acid. When triplex molecules interact with a target region, a structure called a triplex is formed in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a Kd less than 10-6, 10-8, 10-10, or 10-12.

External Guide Sequences

The functional nucleic acids can be external guide sequences. External guide sequences (EGSs) are molecules that bind a target nucleic acid molecule forming a complex, which is recognized by RNase P, which then cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukaryotic cells. Representative examples of how to make and use EGS molecules to facilitate cleavage of a variety of different target molecules are known in the art.

RNA Interference

In some embodiments, the functional nucleic acids induce gene silencing through RNA interference (siRNA). Expression of a target gene can be effectively silenced in a highly specific manner through RNA interference.

Gene silencing was originally observed with the addition of double stranded RNA (dsRNA) (Fire, et al. (1998) Nature, 391:806-11; Napoli, et al. (1990) Plant Cell 2:279-89; Harmon, (2002) Nature, 418:244-51). Once dsRNA enters a cell, it is cleaved by an RNase III-like enzyme called Dicer, into double stranded small interfering RNAs (siRNA) 21-23 nucleotides in length that contain 2 nucleotide overhangs on the 3′ ends (Elbashir, et al., Genes Dev., 15:188-200 (2001); Bernstein, et al., Nature, 409:363-6 (2001); Hammond, et al., Nature, 404:293-6 (2000); Nykanen, et al., Cell, 107:309-21 (2001); Martinez, et al., Cell, 110:563-74 (2002)). The effect of iRNA or siRNA or their use is not limited to any type of mechanism.

In one embodiment, a siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme dicer (Elbashir, et al., Nature, 411:494-498 (2001)) (Ui-Tei, et al., FEBS Lett, 479:79-82 (2000)). siRNA can be chemically or in vitro-synthesized or can be the result of short double-stranded hairpin-like RNAs (shRNAs) that are processed into siRNAs inside the cell. For example, WO 02/44321 describes siRNAs capable of sequence-specific degradation of target mRNAs when base-paired with 3′ overhanging ends, herein incorporated by reference for the method of making these siRNAs. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit.

Therefore, in some embodiments, the composition includes a vector expressing the siRNA. The production of siRNA from a vector is more commonly done through the transcription of a short hairpin RNAse (shRNAs). Kits for the production of vectors including shRNA are available, such as, for example, Imgenex's GENESUPPRESSOR™ Construction Kits and Invitrogen's BLOCK-IT™ inducible RNAi plasmid and lentivirus vectors. In some embodiments, the functional nucleic acid is siRNA, shRNA, or miRNA.

c. Transcription Terminator

Transcriptional terminators used in the described expression vectors can include any terminator sequences known in the art. For example, if the nucleic acid sequence is DNA, the eukaryotic transcription terminator may be TAA, TGA or TAG. If RNA is used as the nucleic acid sequence, the eukaryotic transcription terminator may be UAA, UGA or UAG.

d. Origin or Replication

Prokaryotic origins of replication used in the described expression vectors can include any known in the art. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

A non-limiting list of exemplary sequences include those of the plasmids pMB1, pUC, ColE1, p15A, pSC101, R1, RK2, RF6, F1, M13, lambda, pA81, pRAS3.1, pTi, pBPS1, pUOl, pKH9, pWKS1, pCD1, pMAK3, pBL63.1, pTA1060, p4M, pHT926, pCD6, pJB01, pLME300, pMD5057, pTE44, pDP1 and pT38.

e. Selectable Markers

The described expression plasmids can be engineered to include one or more selection markers, suitable for the needs of the hybrid vector. Any selectable markers known in the art can be used, such as antibiotic resistance genes. Exemplary antibiotic resistance genes include those which impart resistance to ampicillin, kanamycin, neomycin, chloramphenicol, gentamycin, tetracycline, erthyromycin, vancomycin, spectinomycin and streptomycin, and combinations thereof.

4. Pore-Forming Proteins and Enzymes

Compositions for promoting egress from the endosome and/or lysosome of cell, such as an eukaryotic cell, and preventing or reducing degradation of nucleic acid and protein payloads are described.

Proposed mechanisms for the uptake of hybrid bio-synthetic gene delivery vectors by antigen presenting include cells size- and receptor-mediated encapsulation of delivery vectors, followed by general acidification through the fusion of lysosomes (except in the caveolar-mediated pathway). Generally, receptor-mediated internalization of hybrid-vectors by eukaryote cells results in encapsulation of the particles by phagsomes or phagosome-lysosomes (secondary lysosomes) that are formed upon particle uptake by mammalian cells. These intracellular compartments are then acidified. The phagsome or phagosome-lysosome mediated encapsulation of delivery vehicles following cellular uptake represents a significant hurdle to the effective delivery of nucleic acids and other payloads to the cell cytoplasm.

Preferably, the described vectors possess innate escape mechanisms to aid the unpackaging and release of pDNA into the cytosol upon acidification, for eventual nuclear translocation and expression. For example, polymeric components mediate release through a charge-related proton mechanism termed the “Proton Sponge” effect (Pack, et al., Nat Rev Drug Discov. 4:581-93 (2005)). However, due to the duality of the hybrid vectors, there is an additional biologically-based escape mechanism that mediates independent release. This biological escape mechanism can be, in one embodiment, a non-native pore-forming peptide, listeriolysin O, which can be engineered and specifically introduced into the hybrid bacterial core.

Thus, the bacteria hybrid vectors include or can express one or more compounds that assist egress from phagsomes or phagosome-lysosomes of eukaryote host cells (secondary lysosomes) following uptake by the cell. The exogenous cargo can be released and/or expressed within the host cell, for example, to be processed and presented by antigen-presenting cells to initiate an adaptive immune response. When the gene that encodes for a pore-forming protein or lytic enzyme is expressed, the sub-cellular is perforated, providing an escape mechanism from phagsomes or phagosome-lysosomes (secondary lysosomes) that are formed upon particle uptake by mammalian cells.

The gene that encodes the pore-forming protein may be present within chromosomal or extra-chromosomal (e.g. plasmid) DNA. Suitable genes that encode for a pore-forming protein include hly (listeriolysin O [LLO]), ilo (Ivanolysin), slo (Streptolysin O), ply (Pneumolysin), pfoA (Perfringolysin O). For example, the bacterial-component of the hybrid vector can be designed to heterologously express pH-dependent hemolysin protein, LLO. LLO can increase degradative activity, leading to increased resulting gene delivery and/or responses.

Therefore, in certain embodiments, the gene that encodes for the pore-forming protein is present within a plasmid, the plasmid is comprised of a prokaryotic promoter, a nucleic acid encoding the amino-acid sequence of a pore-forming protein downstream of and operably linked to said promoter, a prokaryotic origin of replication and optionally, a selectable marker.

a. Lysteriolysin

In certain embodiments, the protein that assists in phagosomal escape is Listeriolysin O (LLO). Listeriolysin O is a sulfhydryl-activated pore-forming protein produced by the bacterium Listeria monocytogenes that enables the escape of the bacterium from phagosomal vacuoles and entry into the cytosol of host cells. After binding to target membranes within the phagosome of the host, the LLO protein undergoes conformation changes that give rise to insertion within the host cell membrane and subsequently the formation of an oligomeric complex that creates a pore through the membrane. The cytolytic activity of LLO is pH-dependent. Activity is high in the acidic environment of the phagosome, such that LLO permeabilizes the host cell membrane at the optimum pH of 5.5, but has no detectable activity in the neutral pH of the cytosol (Domann and Chakraborty, Nucleic Acids Res. 17:6406-6406 (1989))

Lysosomal escape of bacterial hybrid vectors can be engineered through the heterologous expression of a pore-forming listeriolysin O (LLO) protein for cytoplasmic release of genetic cargo (Radford, et al., Gene Ther., 9, 1455-1463 (2002); Higgins, et al., Mol Microbiol, 31, 1631-41(1999); Parsa, et al., J. Biotechnol., 137, (1-4), 59-64 (2008); Critchley, et al., Gene Ther, 11, (15), 1224-3318, (2004); Grillot-Courvalin, et al., Cellular Microbiology, 4, (3), 177-8619 (2002)).

Nucleic acid sequences for the listeriolysin O gene product are known in the art. See, for example, gen bank ID: CAA42639.1, which provides the nucleic acid sequence:

```
(SEQ ID NO: 1)
                10 20 30 40 50
TAACGACGAT AAAGGGACAG CAGGACTAGA ATAAAGCTAT AAAGCAAGCA
                60 70 80 90 100
TATAATATTG CGTTTCATCT TTAGAAGCGA ATTTCGCCAA TATTATAATT
              110 120 130 140 150
ATCAAAAGAG AGGGGTGGCA AACGGTATTT GGCATTATTA GGTTAAAAAA
              160 170 180 190 200
TGTAGAAGGA GAGTGAAACC CATGAAAAAA ATAATGCTAG TTTTTATTAC
              210 220 230 240 250
ACTTATATTA GTTAGTCTAC CAATTGCGCA ACAAACTGAA GCAAAGGATG
              260 270 280 290 300
CATCTGCATT CAATAAAGAA AATTCAATTT CATCCATGGC ACCACCAGCA
              310 320 330 340 350
TCTCCGCCTG CAAGTCCTAA GACGCCAATC GAAAAGAAAC ACGCGGATGA
              360 370 380 390 400
AATCGATAAG TATATACAAG GATTGGATTA CAATAAAAAC AATGTATTAG
              410 420 430 440 450
TATACCACGG AGATGCAGTG ACAAATGTGC CGCCAAGAAA AGGTTACAAA
              460 470 480 490 500
GATGGAAATG AATATATTGT TGTGGAGAAA AAGAAGAAAT CCATCAATCA
              510 520 530 540 550
AAATAATGCA GACATTCAAG TTGTGAATGC AATTTCGAGC CTAACCTATC
              560 570 580 590 600
CAGGTGCTCT CGTAAAAGCG AATTCGGAAT TAGTAGAAAA TCAACCAGAT
              610 620 630 640 650
GTTCTCCCTG TAAAACGTGA TTCATTAACA CTCAGCATTG ATTTGCCAGG
              660 670 680 690 700
TATGACTAAT CAAGACAATA AAATCGTTGT AAAAAATGCC ACTAAATCAA
              710 720 730 740 750
ACGTTAACAA CGCAGTAAAT ACATTAGTGG AAAGATGGAA TGAAAAATAT
              760 770 780 790 800
GCTCAAGCTT ATCCAAATGT AAGTGCAAAA ATTGATTATG ATGACGAAAT
              810 820 830 840 850
GGCTTACAGT GAATCACAAT TAATTGCGAA ATTTGGTACA GCATTTAAAG
              860 870 880 890 900
CTGTAAATAA TAGCTTGAAT GTAAACTTCG GCGCAATCAG TGAAGGGAAA
              910 920 930 940 950
ATGCAAGAAG AAGTCATTAG TTTTAAACAA ATTTACTATA ACGTGAATGT
              960 970 980 990 1000
TAATGAACCT ACAAGACCTT CCAGATTTTT CGGCAAAGCT GTTACTAAAG
            1010 1020 1030 1040 1050
AGCAGTTGCA AGCGCTTGGA GTGAATGCAG AAAATCCTCC TGCATATATC
            1060 1070 1080 1090 1100
TCAAGTGTGG CGTATGGCCG TCAAGTTTAT TTGAAATTAT CAACTAATTC
            1110 1120 1130 1140 1150
CCATAGTACT AAAGTAAAAG CTGCTTTTGA TGCTGCCGTA AGCGGAAAAT
            1160 1170 1180 1190 1200
CTGTCTCAGG TGATGTAGAA CTAACAAATA TCATCAAAAA TTCTTCCTTC
            1210 1220 1230 1240 1250
AAAGCCGTAA TTTACGGAGG TTCCGCAAAA GATGAAGTTC AAATCATCGA
            1260 1270 1280 1290 1300
CGGCAACCTC GGAGACTTAC GCGATATTTT GAAAAAAGGC GCTACTTTTA
            1310 1320 1330 1340 1350
ATCGAGAAAC ACCAGGAGTT CCCATTGCTT ATACAACAAA CTTCCTAAAA
            1360 1370 1380 1390 1400
GACAATGAAT TAGCTGTTAT TAAAAACAAC TCAGAATATA TTGAAACAAC
            1410 1420 1430 1440 1450
TTCAAAAGCT TATACAGATG GAAAAATTAA CATCGATCAC TCTGGAGGAT
            1460 1470 1480 1490 1500
ACGTTGCTCA ATTCAACATT TCTTGGGATG AAGTAAATTA TGATCCTGAA
            1510 1520 1530 1540 1550
GGTAACGAAA TTGTTCAACA TAAAAACTGG AGCGAAAACA ATAAAAGCAA
            1560 1570 1580 1590 1600
GCTAGCTCAT TTCACATCGT CCATCTATTT GCCAGGTAAC GCGAGAAATA
            1610 1620 1630 1640 1650
TTAATGTTTA CGCTAAAGAA TGCACTGGTT TAGCTTGGGA ATGGTGGAGA
            1660 1670 1680 1690 1700
ACGGTAATTG ATGACCGGAA CTTACCACTT GTGAAAAATA GAAATATCTC
            1710 1720 1730 1740 1750
CATCTGGGGC ACCACGCTTT ATCCGAAATA TAGTAATAAA GTAGATAATC
            1760 1770 1780 1790 1800
CAATCGAATA ATTGTAAAAG TAATAAAAAA TTAAGAATAA AACCGCTTAA
            1810 1820 1830 1840 1850
CACACACGAA AAAATAAGCT TGTTTTGCAC TCTTCGTAAA TTATTTTGTG
            1860 1870 1880 1890 1900
AAGAATGTAG AAACAGGCTT ATTTTTTAAT TTTTTTAGAA GAATTAACAA
            1910 1920 1930 1940 1950
ATGTAAAAGA ATATCTGACT GTTTATCCAT ATAATATAAG CATATCCCAA
            1960 1970 1980 1990 2000
AGTTTAAGCC ACCTATAGTT TCTACTGCAA AACGTATAAT TTAGTTCCCA
            2010 2020 2030 2040 2048
CATATACTAA AAAACGTGTC CTTAACTCTC TCTGTCAGAT TAGTTGTA.
```

Nucleotide sequences that have at least 80%, 85%, 90%, 95%, 99% or 100% amino acid sequence identity to SEQ ID NO: 1 are also described.

The listeriolysin O polypeptide is a 521 amino acid cytoplasmic protein with a molecular weight of approximately 58,688 Amino acid sequences of the listeriolysin O protein are known in the art. See, for example, GenBank Accession No. P13128, which provides the amino acid sequence:

```
(SEQ ID NO: 2)
                10 20 30 40 50
MKKIMLVFIT LILVSLPIAQ QTEAKDASAF NKENSISSMA PPASPPASPK
                60 70 80 90 100
TPIEKKHADE IDKYIQGLDY NKNNVLVYHG DAVTNVPPRK GYKDGNEYIV
              110 120 130 140 150
VEKKKKSINQ NNADIQVVNA ISSLTYPGAL VKANSELVEN QPDVLPVKRD
              160 170 180 190 200
SLTLSIDLPG MTNQDNKIVV KNATKSNVNN AVNTLVERWN EKYAQAYPNV
              210 220 230 240 250
SAKIDYDDEM AYSESQLIAK FGTAFKAVNN SLNVNFGAIS EGKMQEEVIS
              260 270 280 290 300
FKQIYYNVNV NEPTRPSRFF GKAVTKEQLQ ALGVNAENPP AYISSVAYGR
              310 320 330 340 350
QVYLKLSTNS HSTKVKAAFD AAVSGKSVSG DVELTNIIKN SSFKAVIYGG
              360 370 380 390 400
SAKDEVQIID GNLGDLRDIL KKGATFNRET PGVPIAYTTN FLKDNELAVI
              410 420 430 440 450
KNNSEYIETT SKAYTDGKIN IDHSGGYVAQ FNISWDEVNY DPEGNEIVQH
              460 470 480 490 500
KNWSENNKSK LAHFTSSIYL PGNARNINVY AKECTGLAWE WWRTVIDDRN
              510 520
LPLVKNRNIS IWGTTLYPKY SNKVDNPIE.
```

In certain embodiments, the pore-forming protein is a pore-forming variant or fragment of listeriolysin O. Listeriolysin O polypeptides that have at least 80%, 85%, 90%, 95%, 99% or 100% amino acid sequence identity to SEQ ID NO: 2 are described.

The gene encoding LLO or a functional fragment or variant thereof can be inserted at any suitable location within the expression vector to allow for expression of the LLO gene and production of the LLO protein. For example, the gene encoding for LLO may be inserted at the clp location.

b. Endolysin

In certain embodiments, the pore-forming protein is Endolysin, or a pore-forming variant or fragment of Endolysin. Endolysins are hydrolytic enzymes produced by bacteriophages to break apart the cell wall of the host bacterium during the final stage of the lytic cycle. Lysins enzymes target one of the five bonds within peptidoglycan (murein), the principal component of bacterial cell walls. The catalytic domain digests peptidoglycan at a high rate, giving rise to holes in the bacterial cell wall which cause effective lysis of the bacteria.

Therefore, lysosomal escape of bacterial hybrid vectors can be engineered through the heterologous expression of a pore-forming endolysin that forms transmembrane tunnels through the bacterial envelope enzyme for cytoplasmic release of genetic cargo. It may be that these compromised bacterial vectors may experience further membrane destabilization upon lysosomal entrapment, facilitating increased release of pDNA and the pore-forming protein, if present.

The endolysin enzymes can be specific for a strain of bacteria and do not affect the cytoplasmic membrane of eukaryotes. The gene that encodes for an endolysin or a catalytic domain thereof may be present within chromosomal or extra-chromosomal (e.g. plasmid) DNA. Exemplary genes that encode for endolysins include, for example, bacteriophage DX174 Lysis gene E (LyE) and those from Groups 1-13.

In a preferred embodiment, the gene that encode for endolysins is LyE. Therefore, in some embodiments where the gene for the endolysin or catalytic domain thereof is present within plasmids, the plasmid is comprised of a prokaryotic promoter, an endolysin- (or catalytic subunit thereof) coding nucleic acid sequence downstream of and operably linked to the promoter, a prokaryotic origin of replication and optionally, a selectable marker. The promoter may be a constitutive promoter or it may be a regulated promoter.

The bacteriophage ΦX174 Lysis gene E (LyE) polypeptide is a 91 amino acid cytoplasmic protein. Nucleic acid sequences of the bacteriophage ΦX174 Lysis gene E (LyE) are known in the art. See, for example, GenBank Accession No. CAA84691.1 (embl accession Z35638.1), which provides the nucleic acid sequence:

```
(SEQ ID NO: 3)
                10 20 30 40 50
ATGGTACGCT GGACTTTGTG GGATACGCTA GCTTTCCTGC TCCTGTTGAG
                60 70 80 90 100
TTTATTGCTG CCAAGCTTGC TGATCATGTT CATCCCGTCG ACATTCAAAC
              110 120 130 140 150
GGCCGGTGAG CTCATGGAAG GCGCTGAATT TACGGAAAAC ATTATTAATG
              160 170 180 190 200
GCCTCGAGCG TCCGGTTAAA GCCGCTGAAC TGCAGCCGGT TACCTTGCGT
              210 220 230 240 250
GTACGCGCAG GAAACACTGA CGTTCTTACT GACGCAGAAG AAAACGTGCG
              260 270
TCAAAAATTA CGTGCAGAAG GAGTGA.
```

Nucleotide sequences that have at least 80%, 85%, 90%, 95%, 99% or 100% amino acid sequence identity to SEQ ID NO: 3 are also described.

The bacteriophage ΦX174 endolysin polypeptide is a 91 amino acid cytoplasmic protein with a molecular weight of approximately 10,602 Da. Amino acid sequences of the bacteriophage ΦX174 endolysin protein are known in the art. See, for example, GenBank Accession No. P03639, which provides the amino acid sequence:

```
(SEQ ID NO: 4)
                10 20 30 40 50
MVRWTLWDTL AFLLLLSLLL PSLLIMFIPS TFKRPVSSWK ALNLRKTLLM
                60 70 80 90
ASSVRLKPLN CSRLPCVYAQ ETLTFLLTQK KTCVKNYVRK E.
```

In certain embodiments, the pore-forming protein is a pore-forming variant or fragment of endolysin. Polypeptides that have at least 80%, 85%, 90%, 95%, 99% or 100% amino acid sequence identity to SEQ ID NO: 4 are also described.

B. Excipients, Delivery Vehicles and Devices

Hybrid bacterial vectors can be administered and taken up into the cells of a subject with or without the aid of a delivery vehicle. Appropriate delivery vehicles for the described hybrid vectors are known in the art and can be selected to suit the needs of the desired purpose. In a preferred embodiment, the hybrid vector is delivered by injection intravenously, subcutaneously, intraperitoneally, or locally. Typical carriers are saline, phosphate buffered saline, and other injectable carriers.

Formulations including hybrid bacterial vectors with or without delivery vehicles are described. The hybrid bacterial vectors can be formulated into pharmaceutical compositions including one or more pharmaceutically acceptable carriers. Pharmaceutical compositions can be formulated for different mechanisms of administration, according to the desired purpose of the hybrid vectors and the intended use. Pharmaceutical compositions formulated for administration by parenteral (intramuscular, intraperitoneal, intravenous (IV) or subcutaneous injection), topical or transdermal (either passively or using iontophoresis or electroporation) routes of administration or using bioerodible inserts are described.

1. Parenteral Administration

In some embodiments, hybrid bacterial vectors are formulated for administration in an aqueous solution, by parenteral injection. The formulation may also be in the form of a suspension or emulsion. In general, pharmaceutical compositions are provided including effective amounts of an active agent, targeting moiety, and optional a delivery vehicle and optionally include pharmaceutically acceptable diluents, preservatives, solubilizers, emulsifiers, adjuvants and/or carriers. Such compositions include the diluents sterile water, buffered saline of various buffer content (e.g., Tris-HCl, acetate, phosphate), pH and ionic strength and optionally additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Pulmonary and Mucosal Administration

In further embodiments hybrid bacterial vectors are formulated for administration to the mucosa, such as the mouth, eyes, lungs, nasal, oral (sublingual, buccal), vaginal, or rectal mucosa.

Formulations for administration to the mucosa will typically be spray dried drug particles, which may be incorporated into a tablet, gel, capsule, suspension or emulsion. Standard pharmaceutical excipients are available from any formulator.

In one embodiment, the compounds are formulated for pulmonary delivery, such as intranasal administration or oral inhalation. The respiratory tract is the structure involved in the exchange of gases between the atmosphere and the blood stream. The upper and lower airways are called the conducting airways. The terminal bronchioli divide into respiratory bronchiole, which then lead to the ultimate respiratory zone, the alveoli, or deep lung. The deep lung, or alveoli, is the primary target of inhaled therapeutic aerosols for systemic drug delivery. Therapeutic agents that are active in the lungs can be administered systemically and targeted via pulmonary absorption. The term aerosol as used herein refers to any preparation of a fine mist of particles, which can be in solution or a suspension, whether or not it is produced using a propellant. Aerosols can be produced using standard techniques, such as ultra-sonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration.

Compositions can be delivered to the lungs while inhaling and traverse across the lung epithelial lining to the blood stream when delivered either as an aerosol or spray dried particles having an aerodynamic diameter of less than about 5 microns.

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits. Formulations for pulmonary delivery include unilamellar phospholipid vesicles, liposomes, or lipoprotein particles. Formulations and methods of making such formulations containing nucleic acid are well known to one of ordinary skill in the art. A wide range of mechanical devices designed for pulmonary delivery of therapeutic products can be used, including but not limited to nebulizers, metered dose inhalers, and powder inhalers, all of which are familiar to those skilled in the art.

### IV. Methods of Use

Methods of using the hybrid bacterial vectors are provided. It has been established that bacterial hybrid vectors including positively charged cationic polymers associated with the outer surface of negatively charged prokaryote cells, provide a safe and effective positively charged vector for the delivery of nucleic acids and/or polypeptides to antigen presenting cells. For example, the vectors exploit the inherent immunogenicity of prokaryote cells to facilitate and promote phagocytic uptake by antigen presenting cells, leading to internalization within the phagosome and/or lysososmal compartments of the antigen-presenting cell. The acidic environment of the phagosome and/or lysososmal compartments leads to acidification and degradation of the vector, and provides a permissive environment for the activity of pore-forming proteins, such as listeriolysin O (LLO), to form a channel to facilitate egress of vector components from the phagosome, releasing nucleic acid contents into the phagosome. Subsequent rupture of the phagosome by endolysin enzymes facilitates the release of nucleic acids into the cytoplasm of the antigen-presenting cell. When the nucleic acids and proteins are trans-located to the nucleus of the cell, exogenous genes can be expressed, giving rise to biological effector functions, such as immune modulation.

Therefore, methods of using bacterial hybrid vectors can include administering to the cells of a subject an effective amount of a composition including hybrid bacterial vectors to deliver one or more exogenous genes or polypeptides to the cells of a subject. Typically, the cells are professional antigen-presenting cells.

The bacterial hybrid vectors can induce a biological effect in the cells of the recipient, such as an immune-modulatory effect. For example, bacterial hybrid vectors can be used to stimulate an immune response to a desired antigen in the subject.

In some embodiments, the methods can prevent, reduce, or inhibit the expression or function of a target gene in the subject. In some embodiments, the bacterial hybrid vectors are safe and effective vaccine vectors that serve as an immunogen for eliciting an immune response against a disease.

A. Methods of Delivering Nucleic Acids and Polypeptides

It has been established that hybrid bacterial vectors enhance the delivery of genes to target cells as a function of combining the capabilities of the biological and biomaterial components of the overall vector. It may be that cationic polymers at the surface of the hybrid bacterial vectors are internalized into the cell by generalized endocytosis. In certain embodiments, where specialized receptors are grafted to the prokaryotic cell surface as targeting ligands, the cationic polymers may be internalized into the prokaryotic cell through mechanism mediated by the specialized receptors. Following internalization, cationic polymers can mediate escape from the lysosome of target cells by the “proton-sponge effect” (Jones, et al., Mol Pharm, 10, (11), 4082-4098 (2013); Pack, et al. Nat Rev Drug Discov, 4, (7), 581-93 (2005)).

The hybrid bacterial vectors can deliver exogenous nucleic acids and polypeptides to eukaryote cells in vivo or in vitro. The delivery requires contact and internalization of the hybrid bacterial vectors by the target cells. Internalization can occur through one or more different mechanisms. The contacting between the hybrid bacterial vectors and target cells can be induced occur in vivo or in vitro. Typically, the contacting occurs in vivo.

Therefore, in some embodiments, the compositions of hybrid bacterial vectors are administered systemically to a subject. In other embodiments, the hybrid bacterial vectors are directly administered to a specific bodily location of the subject. In further embodiments, the route of administration targets the hybrid vectors directly to a specific organ.

Pharmaceutical compositions including hybrid bacterial vectors can be administered in a variety of manners, depending on whether local or systemic administration is desired, and depending on the area to be treated. For example, the described compositions can be administered intravenously, intraperitoneally, intramuscularly, subcutaneously, intracavity, or transdermally. The compositions may be administered parenterally (e.g., intravenously), by intramuscular injection, by intraperitoneal injection, transdermally, extracorporeally, topically or the like, including topical intranasal administration or administration by inhalation.

Parenteral administration of the composition, if used, is generally characterized by injection. Injectable formulations can be prepared in conventional forms, either as liquid solutions or suspensions, solid forms suitable for solution of suspension in liquid prior to injection, or as emulsions. Administration involving use of a slow release or sustained release system, such that a constant dosage is maintained, is also discussed.

In certain embodiments, the compositions are administered locally, for example, by injection directly into a site to be treated. In some embodiments local delivery can reduce side effects or toxicity associated with systemic delivery and can result in enhanced outcome due to an increased localized dose.

Methods of administering the hybrid bacterial vectors locally (i.e., via local drug delivery, LDD) are provided. In certain embodiments, hybrid bacterial vectors can be administered directly to a treated tissue, such as an artery or vein, without engendering adverse systemic effects. In further embodiments, the compositions are injected or otherwise administered directly to one or more surgical sites. Typically, local injection causes an increased localized concentration of the compositions which is greater than that which can be achieved by systemic administration.

In some embodiments hybrid bacterial vectors delivered locally result in concentrations that are twice, 10 times, 100 times, 500 times, 1000 times or more than 1000 times greater than that achieved by systemic administration.

In some embodiments systemically administered hybrid bacterial vectors persist in the blood stream and release the cargo to target cells over a period of time. Preferably, the steady release maintains a desired concentration of exogenous nucleic acids or polypeptides in the target cells.

The hybrid bacterial vectors can be administered during a period before, during, or after onset of symptoms of a disease, or any combination of periods before, during or after onset of one or more disease symptoms. For example, the subject can be administered one or more doses of the composition every 1, 2, 3, 4, 5, 6 7, 14, 21, 28, 35, or 48 days prior to onset of disease symptoms. The subject can be administered one or more doses of the composition every 1, 2, 3, 4, 5, 6, 7, 14, 21, 28, 35, or 48 days after the onset of disease symptoms. In some embodiments, the multiple doses of the compositions are administered before an improvement in disease condition is evident. For example, in some embodiments, the subject receives 1, 2, 3, 4, 5, 6 7, 14, 21, 28, 35, or 48, over a period of 1, 2, 3, 4, 5, 6 7, 14, 21, 28, 35, or 48 days or weeks before an improvement in the disease or condition is evident.

Thus, compositions including one or more hybrid bacterial vectors can be administered at different times in relation to a diagnosis, prognosis, surgery or injury depending on the desired effects of the nucleic acids or polypeptides that are delivered to the target cells. The timing of commencement of administration of the hybrid bacterial vectors should be determined based upon the needs of the subject, and can vary accordingly. In some embodiments a single dose of hybrid bacterial vectors is delivered to a subject as one or more bolus doses to raise the blood concentration of the hybrid vectors, or the blood concentration of the payload of the hybrid vectors to a desired level. The bolus can be given by any means, such as via injection. The placement of the bolus dose can be varied depending upon the desired effect and the target organ or tissue to be treated. In a particular embodiment, a bolus is given prior to the administration of other dosage forms.

For example, the hybrid bacterial vectors can be engineered to impart different residency times in the blood stream, for example, by modification of one or more of targeting moieties, pegylation, polymer density, etc. Thus, the desired blood concentration of hybrid bacterial vectors can be maintained for a desired period of time using a combination of formulations, administered at the same time, or as a series of administrations over a period of time, as desired.

B. Vaccination

In some embodiments, bacterial hybrid vectors can deliver exogenous proteins and/or nucleic acids to antigen presenting cells (APC) of a subject to stimulate desired immune responses in the subject. The low cytotoxicity of the bacterial hybrid vectors makes them particularly attractive for use as part of a vaccine.

Experiments conducted with the bacterial hybrid vectors including the OVA peptide, discussed below, show bacterial hybrid vectors generate a strong immune response against the virus, and also against the OVA peptide. Accordingly, other proteins could be substituted for OVA. These could include proteins from pathogenic microbes unrelated to the bacterial hybrid vectors; the bacterial hybrid vectors could serve as a safe vaccine platform against many different pathogenic microbes. As described above, bacterial hybrid vectors can be engineered to express one or more exogenous immunogenic antigens.

Delivery of exogenous antigen to APC (e.g., macrophages) of a subject results in the production of antibodies and other biomolecules capable of recognizing and neutralizing the antigen. Antigen that has been arrayed on the surface of antigen-presenting cells (APC) can be presented to a “helper” T cell, such as an antigen-specific naive CD4+ T cell. Such presentation delivers a signal via the T cell receptor (TCR) that directs the T cell to initiate an immune response that will be specific to the presented.

Therefore, the bacterial hybrid vectors can be used to initiate, moderate or enhance a humoral and/or cellular immunity to an encoded antigen. For example, the hybrid bacterial vectors deliver exogenous nucleic acids and/or proteins in an amount effective to induce, enhance or otherwise moderate the biological activities of immune cells, such as macrophages, B-cells, T-cells, dendritic cells and NK cells.

In some embodiments, administration of the bacterial hybrid vectors including nucleic acid sequences encoding an antigen to a subject confers immunity to the antigen to the subject Immunity can manifest in the production of a reservoir of memory T cells (i.e., memory CD8+ T cells) and/or antigen-specific B cells in the subject sufficient to provide rapid immune cellular and/or humoral immune responses to repeat exposure of the antigen. Preferably, administration of the bacterial hybrid vectors including nucleic acid sequences encoding an antigen confers protection against infection or disease caused by the organism(s) from which the antigen is derived.

Typically, administration of the bacterial hybrid vectors including nucleic acid sequences encoding an antigen to a subject enhances the uptake and delivery of antigen to the antigen presenting cells of a subject relative to administration of equal amounts of the antigen or nucleic acid encoding the antigen alone. Therefore, administration of antigen to a subject via the described bacterial hybrid vectors can enhance the immune response to the antigen in the subject relative to administration of equal amounts of the antigen or nucleic acid encoding the antigen alone. For example, bacterial hybrid vectors can increase, prolong or otherwise enhance presentation of the encoded antigen at the surface of antigen presenting cells of the subject.

Vaccines can be administered prophylactically or therapeutically. Vaccines can also be administered according to a vaccine schedule. A vaccine schedule is a series of vaccinations, including the timing of all doses. Many vaccines require multiple doses for maximum effectiveness, either to produce sufficient initial immune response or to boost response that fades over time. Vaccine schedules are known in the art, and are designed to achieve maximum effectiveness. The adaptive immune response can be monitored using methods known in the art to measure the effectiveness of the vaccination protocol.

1. Pathogens/Diseases to be Vaccinated Against

Bacterial hybrid vectors can deliver protein and nucleic acid antigen to APC of a subject in an amount effective to vaccinate the subject from one or more diseases and disorders. The bacterial hybrid vectors can serve as a vaccination platform for a wide variety of microbial pathogens, such as bacterial, viral, fungal and protozoan pathogens.

In some embodiments the target of the vaccine could be a type of cancer cell as a cancer treatment. Alternately, the target could be any of a large number of microbial pathogens. Exemplary diseases that can be vaccinated against include disease for which vaccines are currently available, including Anthrax; Cervical Cancer (Human Papillomavirus); Diphtheria; Hepatitis A; Hepatitis B; Haemophilus influenzae type b (Hib); Human Papillomavirus (HPV); Influenza viruses (Flu); Japanese encephalitis (JE); Lyme disease; Measles; Meningococcal; Monkeypox; Mumps; Pertussis; Pneumococcal; Polio; Rabies; Rotavirus; Rubella; Shingles (Herpes Zoster); Smallpox; Tetanus; Typhoid; Tuberculosis (TB); Varicella (Chickenpox); Yellow Fever.

In some embodiments, hybrid bacterial vectors can be used to immunize a subject against an infectious disease or pathogen for which no alternative vaccine is available, such as diseases including but not limited to, malaria, streptococcus, Ebola Zaire, HIV, Herpes virus, hepatitis C, Middle East Respiratory Syndrome (MERS), Sleeping sickness, Severe Acute Respiratory Syndrome (SARS), rhinovirus, chicken pox, hendra, NIPA virus, and others.

In certain embodiments, the disease is cancer.

In some embodiments, the disease is a pathogen that infects non-mammalian subjects, such as birds. Exemplary avian subjects include domesticated birds (i.e., poultry), such as chickens, ducks, geese, pheasants and other commercial fowl, or pet birds such as parakeets and parrots. For example, bacterial hybrid vectors can be useful to vaccinate birds against Infectious Bursal Disease (IBD). IBD, also known as Gumboro disease, is a viral disease affecting the Bursa of Fabricius of young chickens. Other diseases and disorders of poultry that can be vaccinated for using the described bacterial hybrid vectors include Ranikhet; Mareks disease, fowl pox, fowl cholera, egg drope syndrome, infectious coryza, coccidiosis, avian encephalitis, avian influenza, chicken infectious anemia and salmonella.

Vaccines for poultry are typically administered intranasally, shortly after hatching. Boosters may be administered.

C. Dosages and Effective Amounts

In some in vivo approaches, the compositions of hybrid bacterial vectors are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease or disorder, and the treatment being effected.

For all of the compounds described, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired.

Generally dosage levels of between 0.001 and 100 mg/kg of body weight daily are administered to avians or mammals, most preferably, humans. Generally, for intravenous injection or infusion, dosage may be lower. Preferably, the compositions are formulated to achieve a modified prokaryotic cell serum level of between about 1 and about 1000 μM.

For example, hybrid bacterial vectors can be in an amount effective to deliver antigen to APC and induce the proliferation and clonal expansion of B cells, T cells or induce the migratory or chemotactic activity of macrophages. Therefore, in some embodiments the hybrid bacterial vectors are in an amount effective to stimulate a primary immune response to an antigen in a subject. In a preferred embodiment the effective amount of hybrid bacterial vectors does not induce significant cytotoxicity in the cells of a subject compared to an untreated control subject.

Preferably, the amount of hybrid bacterial vectors is effective to prevent or reduce the infection or onset of a disease or disorder in a subject compared to an untreated control.

In another embodiment, the hybrid bacterial vectors are in an amount effective to decrease the amount of expression of a target gene, or to prevent or decrease the serum concentration of a target gene product in a subject.

In a particular embodiment, hybrid bacterial vectors are in an amount effective to induce presentation of an antigen by antigen presenting cells. For example, hybrid bacterial vectors can be in an amount effective to induce T cell activation in response to an exogenous polypeptide encoded by a gene delivered to antigen presenting cells by the hybrid bacterial vectors. In a further embodiment, the one or more hybrid bacterial vectors are in an amount effective to decrease the amount of antigen required to stimulate a robust or protective immune response to the antigen in a subject. The hybrid bacterial vectors can be effective to induce the production or antibodies to an antigen encoded by the hybrid bacterial vectors.

Thus, hybrid bacterial vectors can be effective to enhance the amount of antigen-specific immune cells in a subject. For example the amount of antigen-specific immune cells in a subject can be increased relative to the amount in an untreated control. For example, hybrid bacterial vectors can be effective to induce several signaling pathways controlling cellular immune activities, including cellular proliferation, chemotaxis and actin reorganization. Preferably the effective amount of hybrid bacterial vectors does not cause cytotoxicity.

D. Controls

The effect of hybrid bacterial vectors can be compared to a control. Suitable controls are known in the art and include, for example, untreated cells or an untreated subject. In some embodiments, the control is untreated tissue from the subject that is treated, or from an untreated subject. Preferably the cells or tissue of the control are derived from the same tissue as the treated cells or tissue. In some embodiments, an untreated control subject suffers from, or is at risk from the same disease or condition as the treated subject. For example, in some embodiments, an untreated control subject does not raise an immune response to an antigen.

E. Combinations

Bacterial hybrid vectors can be administered alone, or in combination with one or more additional active agent(s), as part of a therapeutic or prophylactic treatment regime. The bacterial hybrid vectors can be administered on the same day, or a different day than the second active agent. For example, compositions including bacterial hybrid vectors can be administered on the first, second, third, or fourth day, or combinations thereof.

The term “combination” or “combined” is used to refer to either concomitant, simultaneous, or sequential administration of two or more agents. Therefore, the combinations can be administered either concomitantly (e.g., as an admixture), separately but simultaneously (e.g., via separate intravenous lines into the same subject), or sequentially (e.g., one of the compounds or agents is given first followed by the second).

In some embodiments, the additional prophylactic or therapeutic agents can be vaccines for a specific antigen. The antigen can be the same or different to that encoded by the hybrid bacterial vectors.

In some embodiments the bacterial hybrid vectors are useful as an agent to enhance the immune response to an antigen in a subject relative to the immune response raised to the same antigen in the absence of the bacterial hybrid vectors. Therefore, the bacterial hybrid vectors can act as adjuvants to enhance the uptake and delivery of antigens in the antigen presenting cells of a subject.

### V. Methods of Making

Hybrid bacterial vectors can be formed by the addition of prokaryotic cells and cationic polymers, followed by incubation in an appropriate buffer. An exemplary buffer is sodium acetate at pH 5.0-6.0. Association between bacterial cells and cationic polymers can occur via electrostatic interactions between positively charged polymers and the negatively charged outer membrane of the cell. Therefore, the hybrid bacterial vectors can be formed by the use of simple mixing schemes that bring bacterial cells into contact with the polymers. “Bringing into contact,” as used herein, refers to causing or allowing compounds, compositions, components, materials, etc. to be in contact with each other. As an example, mixing two components into the same solution constitutes bringing the components into contact. Examples of bringing into contact include adding, combining, and mixing cationic polymers and bacterial cells to enable the cationic polymers to adsorb to the bacterial cell surface. “Polymer adsorption,” as used herein, refers to the adsorption between polymer chains and a particle or particles in water due to an attractive force present. Polymer adsorption is generally considered an irreversible process.

Facile methods of formulation enable scalability studies and eliminate complex formulation protocols. Formulations can be prepared using a range of polymer doses, such as between 0.1 mg/ml and 1 mg/ml, for example, 0.25, 0.5, 0.75, and 1.00 mg/mL.

The amount of polymer used in the formulation can directly influence the amount of polymer coated onto each cell. Therefore, the amount of cationic polymer used in formulation of the hybrid bacterial vectors can be varied to modify the structural and functional features of the vector.

The engineering of nucleic acid expression vectors for the expression of exogenous nucleic acids and polypeptides in a prokaryotic or eukaryotic system may be performed by techniques generally known to those of skill in recombinant expression.

Both cDNA and genomic sequences are suitable for eukaryotic expression, as the host cell will generally process the genomic transcripts to yield functional mRNA for translation into protein. It may be more convenient to employ as the recombinant gene a cDNA version of the gene. It is believed that the use of a cDNA version will provide advantages in that the size of the gene will generally be much smaller and more readily employed to transfect the targeted cell than will a genomic gene, which will typically be up to an order of magnitude larger than the cDNA gene.

Many standard techniques are available to construct expression vectors containing the appropriate nucleic acids and transcriptional/translational control sequences in order to achieve protein or peptide expression in a variety of host-expression systems. For example, nucleic acid expression vectors including one or more exogenous nucleic acid sequences under the control of one or more promoters can be engineered to express one or more encoded proteins or peptides an in an eukaryotic cell.

To bring a coding sequence “under the control of” a promoter, the 5′ end of the translational initiation site of the reading frame is positioned between about 1 and 50 nucleotides “downstream” of e., 3′ of) the chosen promoter. The “upstream” promoter stimulates transcription of the inserted DNA and promotes expression of the encoded protein.

The cationic polymers can be directly complexed to the outer surface of prokaryotic cells, or they can be mixed with nucleic acid expression vectors and allowed to four′ nucleic acid/polymer aggregates (i.e., polyplexes) prior to complexing with the prokaryotic cells.

The present invention will be further understood by reference to the following non-limiting examples.

### Examples

### Example 1: Bacterial Hybrid Vectors Including Acrylate-Terminated Poly(Neopentyl Glycol Diacrylate-Co-2-Amino-1,3-Propanediol) Enhance Delivery of Nucleic Acids to APC

### Materials and Methods

Preparation of Bacteria/Polymer Hybrid Vectors.

Bacterial and hybrid vectors were prepared from bacterial cultures inoculated at 2% (vol./vol.) from overnight starter cultures. Plasmid selection antibiotics were used as needed during bacterial culture within lysogeny broth (LB) medium. Following incubation at 36° C. and shaking at 250 rpm to an optical density at 600 nm (OD600) of between 0.4 and 0.5, samples were induced with 0.1 mM Isopropyl β-D-1-thiogalactopyranoside (IPTG) for 1 hr at 30° C. Bacteria cells for use in control samples were washed once and standardized to an OD600 value of 0.5 in PBS.

Bacteria cells to be used in hybrid vector formation were washed once and standardized to an OD600 value of 1.0 in 25 mM NaOAc (pH 5.15).

Cationic polymers (CP) were dissolved in chloroform, desiccated (evaporated to dryness under vacuum) and resuspended in 25 mM NaOAc (pH 5.15). CPs and washed bacteria were then added at an equal.

Hybrid vectors (final 0.5 OD600) and control bacterial strains in PBS were incubated at 22° C. for 15 minutes before being diluted into RPMI medium to produce desired Multiplicity of Infection (MOI).

Preparation of Polymer/Nucleic Acid “Polyplex” Vectors.

Polyplexes were generated following previously described protocols (Green, Methods in Molecular Biology, 480:53-63. 2009). Briefly, PBAE CPs (in molar ratios of CP to pDNA sufficient to ensure complete pDNA complexation, e.g., 30:1 to 200:1) in chloroform were added to Eppendorf tubes, evaporated to dryness under vacuum and re-suspension in 25 mM NaOAc (pH 5.15). An equal volume of pDNA in 25 mM NaOAc buffer (pH 5.15) was added and mixed by vortexing for 10 s.

Polymer/pDNA self-assembly was continued for 15 minutes.

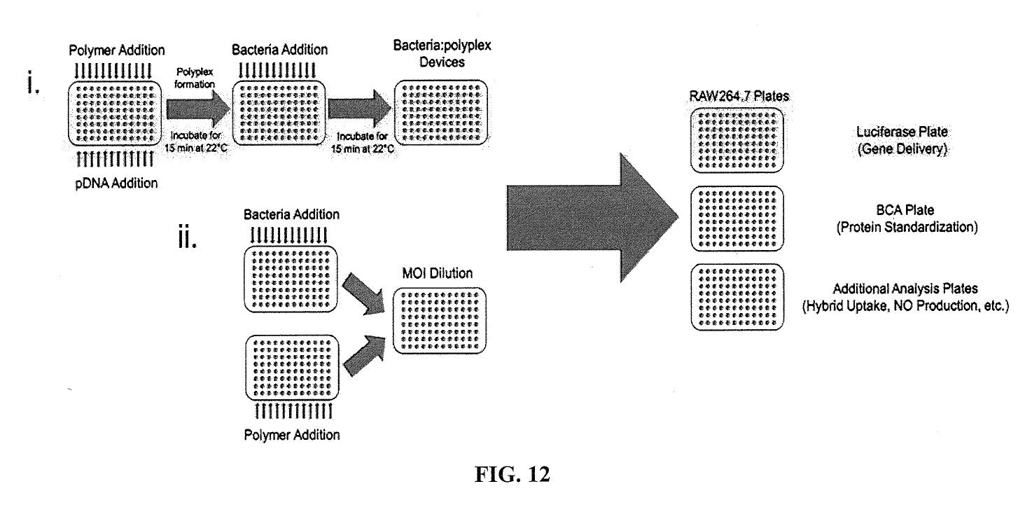

PBAE polyplexes (formulated to deliver 600 ng pCMV-Luc/well) were prepared as described above and used in hybrid device formation (FIG. 12; scheme i). Two hybrid variants were prepared from bacterial vectors designated for hybrid vector formation.

Similarly, a different hybrid variant was prepared by simultaneously diluting bacteria (in PBS) and polymer (in PBS) separately in RPMI-1640 to the desired MOI (FIG. 12; scheme ii).

Instrumentation and Measurements

All 1H-NMR spectra were measured at 500 MHz in CDCl₃ using a Varian INOVA-500 spectrometer maintained at 25° C. with tetramethylsilane (TMS) as an internal reference standard. Gel permeation chromatography (GPC) data were acquired from a Viscotek system equipped with a VE-3580 refractive index (RI) detector, a VE 1122 pump, and two mixed-bed organic columns (PAS-103M and PAS-105M). Dimethylformamide (DMF) containing 0.1 M LiBr was used as the mobile phase with a flow rate of 0.5 mL/min at 55° C. The GPC instrument was calibrated with narrowly dispersed linear polystyrene standards purchased from Varian.

Zeta potential values were obtained using dynamic light scattering (DLS) on a Zetasizer nano-ZS90 (Malvern, Inc.) in 25 mM sodium acetate at 25° C. Experiments were conducted using a 4 mW 633 nm HeNe laser as the light source at a fixed measuring angle of 90° to the incident laser beam and the correlation decay functions were analyzed by the cumulates method coupled with Mie theory.

Scanning electron microscopy (SEM) images were acquired using a Hitachi SU-70 operating at an accelerating voltage of 5.0 kV.

Microplate experiments were analyzed using a Synergy 4 Multi-Mode Microplate Reader (BioTek Instruments, Inc.).

Materials

Monomers were purchased from Sigma-Aldrich (St. Louis, Mo.) and TCI (Portland, Oreg.) (Table 3). Acetone (HPLC), chloroform (HPLC), n-hexadecane (99%), DMF (HPLC), and DMSO (≧99.7%) were purchased from Fisher Chemical. D-(+)-mannose (cell culture grade), 4-toluenesulfonyl chloride (p-TsCl), allyl alcohol (≧99%), hexamethyldisilazane (HMDS; ≧99%), and polymyxin B sulfate (cell culture grade) were purchased from Sigma-Aldrich. Phosphate buffered saline (PBS) and trypan blue solution (0.4% w/v in PBS) were purchased from Life Technologies (Grand Island, N.Y.). Glutaraldehyde (25% in H₂O) was purchased from Polysciences (Warrington, Pa.). Polycarbonate isopore membranes (0.45 μm) and a Swinnex filter holder (25 mm) were purchased from Millipore (Billerica, Mass.).

Polymer Synthesis

Synthesis of cationic polymers was conducted following previous protocols (Schmidt, Nature Biotechnology, 31(11):957-960(2013); Wirth Gene, 525(2):162-169(2013)). Briefly, polymers were synthesized by single step Michael addition at either (1) 95° C. solvent free or (2) 60° C. in 2 mL DMSO. High-temperature synthesis was performed for one day and low-temperature synthesis was performed for five days in a glass vial under magnetic stirring. Unreacted monomers were removed by dialysis against acetone using molecular porous membrane tubing (Spectra/Por Dialysis Membrane, Spectrum Laboratories Inc.) with an approximate molecular weight filter of 3,500 daltons.

Amine/diacrylate stoichiometric ratios were held at 1.2:1 for base polymer syntheses; the diacrylate monomer amount was held constant at 400 mg per reaction.

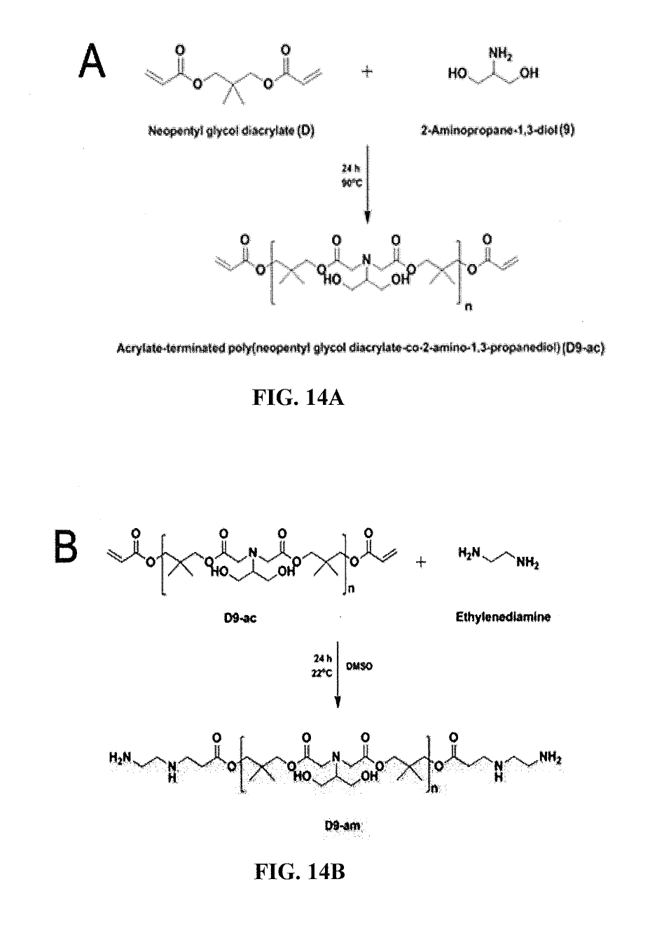

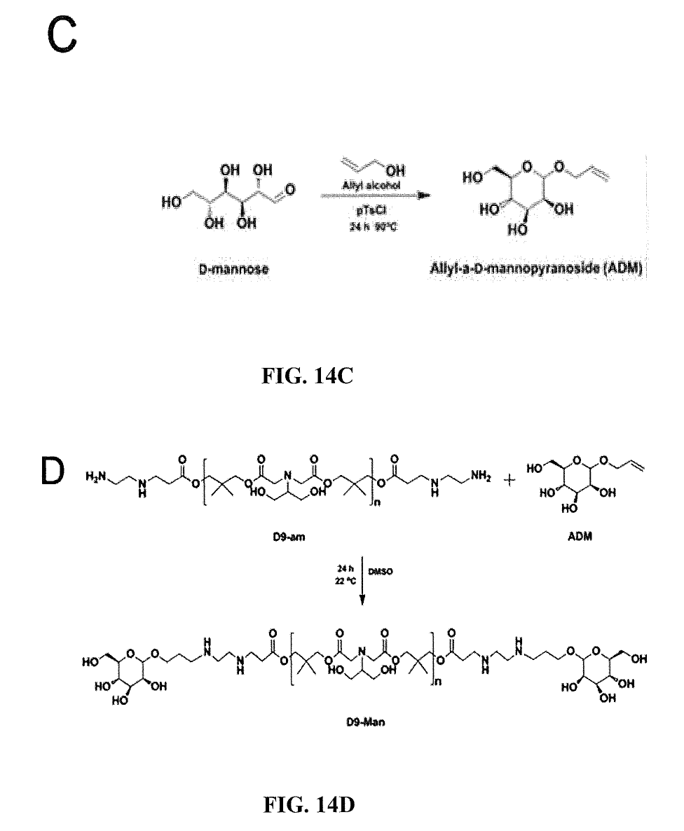

Mannosylated-D9 was synthesized using a three-step procedure (FIGS. 14A-14D). First, acrylate-terminated poly(neopentyl glycol diacrylate-co-2-amino-1,3-propanediol), D9ac, was synthesized without solvent 5 at a 1:1.2 amine (2-amino-1,3-propanediol):diacrylate (neopentyl glycol diacrylate) molar ratio (using 400 mg amine) at 90° C. for 24 h (FIG. 14A).

As a second step, ethylenediamine was reacted in excess with D9ac to amine-cap the terminal ends (FIG. 14B). Specifically, D9ac was dissolved in DMSO at 167 mg/mL and 500 μL reacted with 500 μL of 0.5 M ethylenediamine (in DMSO) at 22° C. for 24 h.

Amine-capped D9 (D9-am) was then reacted with allyl-α-D-mannopyranoside (ADM) at a 1:2 molar ratio in DMSO at 22° C. for 24 h.

Allyl-α-D-mannopyranoside was synthesized by dissolving 3 g of D-mannose and 18 mg p-TsCl in allyl alcohol (20 mL) at 90° C. under reflux for 24 h (FIG. 14D). All polymers were purified by dialysis.

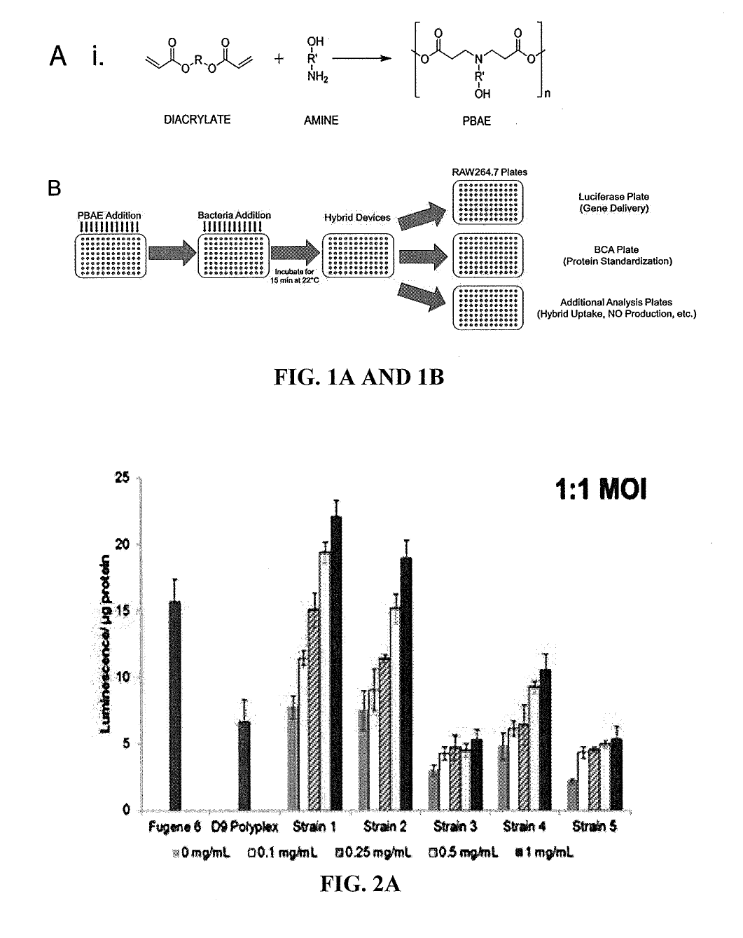

The synthesis of PBAEs proceeded via the conjugate (Michael) addition of amines to acrylate groups. Building upon high-throughput polymer synthesis schemes, a library of 92 PBAEs were produced from a diverse set of monomers (FIGS. 1A-1B; Table 1) (Sunshine, et al., Mol Pharm, 9(11):3375-3383 (2012); Sunshine, et al. Plos One 7(5):e37543 (2012); Anderson, et al., Mol Ther. 11(3):426-434 (2005); Anderson, et al., Angewandte Chemie, 42(27):3153-3158 (2003)).

The library was synthesized on the 1-2 g scale with increased monomer concentrations to provide greater control of stoichiometry, increase polymer molecular weight and end-group termination, and reduce intramolecular cyclization. Each polymer was analyzed using GPC and found to possess a polydispersity index (PDI) and molecular weight of about 1.4 and 5.3 kDa, respectively, consistent with previous reports (Sunshine, et al., Mol Pharm, 9(11):3375-3383 (2012); Green, et al., Acc Chem Res. 41(6):749-759 (2008); Lynn, et al. J. Am. Chem. Soc. 122:10761-10768 (2000)).

Cell Lines, Strains, and Plasmids

A murine RAW264.7 macrophage cell line kindly provided by Dr. Terry Connell (Department of Microbiology and Immunology, University at Buffalo, SUNY) was used for gene delivery assays. The cell line was maintained in medium prepared as follows: 50 ml of fetal bovine serum (heat inactivated), 5 mL of 100 mM MEM sodium pyruvate, 5 mL of 1 M HEPES buffer, 5 mL of penicillin/streptomycin solution, and 1.25 g of D-(+)-glucose were added to 500 mL phenol red-containing RPMI-1640 and filter sterilized. Cells were housed in T75 flasks and cultured at 37° C./5% CO₂.

The BL21(DE3) E. coli cell line (Novagen) was used as the parent strain for generation of all gene delivery bacterial vectors. Genetic manipulations were described previously (Leboulch, Nature, 500(7462):280-282, (2013); Jones, Mol. Pharm 10(11):4082 (2013)). Resulting background strains are listed in Table 2 (with hly being the gene designation of listeriolysin O [LLO]).

A luciferase reporter plasmid driven by a cytomegalovirus (CMV) promoter (pCMV-Luc; Elim Biopharmaceuticals) was utilized during microplate reader-based transfection experiments. Plasmid pRSET-EmGFP was kindly provided by Dr. Sheldon Park (Department of Chemical and Biological Engineering, University at Buffalo, SUNY) and used during uptake studies. An enhanced green fluorescent protein (EGFP) gene driven by a CMV promoter within pCMV-EGFP (Addgene) was used during flow cytometry transfection experiments. The ovalbumin (OVA) gene driven by a CMV promoter within pCI-neo-cOVA (Addgene) was used during mouse immunization experiments. The pCMV-Luc, pCMVEGFP, and pCI-neo-cOVA plasmids were separately transformed into and prepared from GeneHogs (Life Technologies) competent cells prior to use in the experiments outlined below. Plasmid preparation for transfection controls was performed using the PureYield™ Plasmid Midiprep System (Promega).

TABLE 2
Bacterial strains used in production of vectors

| | | | LLO | |
|---|---|---|---|---|
| | | | Expression | |
| Strain | Description | Strain # | Ranking¹ | Ref. |
| BL21(DE3) | E. coli B F⁻ dcm ompT hsdS(r_{B}⁻ m_{B}⁻) gal λ | N/A | N/A | D |
| | (DE3) | | | |
| BL21(DE3)/pCMV- | BL21(DE3) carrying reporter plasmid w/ | 5 | N/A | B |
| Luc | luciferase gene driven by CMV promoter | | | |
| YWT7-hly/pCMV- | Strain contains IPTG-inducible T7-hly | 1 | 2 | A, B |
| Luc | cassette in BL21(DE3) chromosome | | | |
| YWTet-hly/pCMV- | Strain contains constitutive Tet-hly cassette | 2 | 4 | A, B |
| Luc | in BL21(DE3) chromosome | | | |
| BL21(DE3)/pCMV- | pET29-hly carries hly gene under IPTG- | 3 | 1 | A, B, C |
| Luc/ | inducible T7 promoter | | | |
| pET29-hly | | | | |
| BL21(DE3)/pCMV- | pDP3615 carries hly gene under | 4 | 3 | A, B, C |
| Luc/ | constitutive | | | |
| pDP2615 | Tet promoter | | | |
| BL21(DE3)/pCYC- | pCYC-LyE carries LyE gene from | N/A | N/A | This |
| LyE | bacteriophage | | | work |
| YWT7-hly/pCYC- | ΦX174 on IPTG-inducible pACYCDuet | N/A | 2 | This |
| LyE | plasmid | | | work |
| YWT7-hly/pCMV- | | N/A | 2 | This |
| Luc/ | | | | work |
| pCYC-LyE | | | | |
| YWT7-hly/pRSET- | pRSET-EmGFP features IPTG-inducible | N/A | 2 | This |
| EmGFP | emerald | | | work |
| | green fluorescent protein (EmGFP) | | | |
| | expression | | | |
| YWT7-hly/pCMV- | pCMV-EGFP carries enhanced green | N/A | 2 | This |
| EGFP | fluorescent | | | work |
| | protein (EGFP) gene driven by CMV | | | |
| | promoter | | | |
| YWT7-hly/pCI-neo- | pCI-neo-cOVA carries ovalbumin gene | N/A | 2 | This |
| cOVA | driven by | | | work |
| | CMV promoter | | | |

¹Ranking of LLO expression and activity from highest (1) to lowest (4)
Ref A: Mol Pharm, 10(11): 4301-8; Ref B: J. Biotechnol, 137(1-4): 59-64
Ref C: Mol Microbiol, 31: 1631-1641; Ref D: J Mol Biol, 189(1): 113-130.

Transfection Studies

For gene delivery experiments, RAW264.7 cells were seeded into two different types of 96-well plates at 3×10⁴ cells/well in 100 μL antibiotic-free media and incubated for 24 h for attachment; (i) Tissue culture-treated, flat-bottom sterile white polystyrene 96-well plates were used for luciferase assays; and (ii) Tissue culture-treated sterile polystyrene 96-well plates were used for bicinchoninic acid (BCA) assessment (and also 3-(4, 5-dimethylthiazol-2-yl)-diphenyltetrazolium bromide (MTT), EGFP flow cytometry, nitric oxide (NO) production, and bacterial uptake/load assays).

Hybrid, hybrid variant, and bacterial devices were diluted in antibiotic-free RPMI-1640 to desired MOIs. Following cellular attachment, macrophage medium was replaced with 50 μL of each respective vector and allowed to incubate for an hour. After incubation, 50 μL of gentamicin containing RPMI-1640 was added to each well to eliminate external/non-phagocytized vectors. Following an additional 24 h incubation (48 h after initial seeding), plates were analyzed for luciferase expression using the Bright Glo assay (Promega) and protein content using the Micro BCA Protein Assay Kit (Pierce) according to each manufacturer's instructions. Gene delivery was calculated by nottnalizing luciferase expression by protein content for each well/plate.

Flow cytometry experiments for pCMVEGFP transfection were completed using a FACS Calibur flow cytometer (Becton Dickinson, Franklin Lakes, N.J.). Two days after transfection, cells were washed with ice-cold PBS and detached from the well surface using cell scrapers prior to analysis. For proper gating, cells transfected with BL21(DE3) were used as a negative control; whereas, cells separately transfected with each control (Table 2) complexed to pCMV-EGFP were used as positive controls. Results were derived from twelve replicates and four independent experiments.

PBAE polyplexes (formulated to deliver 600 ng pCMV-Luc/well or pCMVEGFP) were added in antibiotic-free RPMI-1640 to the seeded RAW264.7 macrophage cells, and plates were mechanically agitated and incubated for four hours. Polyplex-containing media was then removed using a 12-channel aspirating wand and replaced with 100 μL fresh, antibiotic-free RPMI-1640 5 medium preheated to 36° C. Cells were allowed to incubate for an additional 24 h prior to gene delivery assessment.

Similarly, FuGENE 6 (Promega, Madison, Wis.), FuGENE HD (Promega), ViaFECT (Promega), OmniFect (TransOMIC Technologies, Huntsville, Ala.), Xfect (Clontech Laboratories, Inc., Mountain View, Calif.), JET-PEI (Polyplus-transfection SA, Illkirch, France), and GeneJuice (EMD Millipore) were included as positive controls, complexed to pDNA (Table 2), and analyzed according to each manufacturer's instructions.

Statistical Evaluation

Unless otherwise indicated, all data were generated from three independent experiments. Error bars represent standard deviation values. All statistical significance comparisons between groups were performed using a one-way ANOVA.

### Results

Two normally distinct vectors (a bacterial cell and a synthetic polymer) were combined to generate a hybrid vector.

The hybrid bacterial vector demonstrated synergistic mechanisms in assisting and improving gene delivery to APCs. Furthermore, the unique and complimentary engineering capabilities of the hybrid vector were demonstrated to further tailor and improve APC gene delivery.

Hybrid bio-synthetic vectors that combine the capabilities of both bacterial and CP components were generated for targeted gene delivery to APCs with surprising synergistic results. Ninety-two structurally-diverse PBAEs were synthesized and screened after surface attachment to LLO-producing E. coli for gene delivery to murine macrophages. After multiple rounds of screening, an optimal PBAE and bacterial strain were identified which, when combined together, possessed gene delivery potency greater than either vector in isolation.

Different methods of hybrid device formation were explored where, interestingly, the best performing hybrid contained bacterial surface additions of pDNA-loaded polyplexes.

To demonstrate the engineering potential of the hybrid device, each individual vector was modified using vector-associated tools. Specifically, the lethal lysis gene E (LyE) from bacteriophage ΦX174 was incorporated into bacterial strains which resulted in significant improvements to APC gene delivery and cytotoxicity.

Finally, the hybrid vector was successfully tested in the context of in vivo humoral immune response. The combined features of this new vector offer a platform for applications in genetic vaccination as a byproduct of the duality in vector composition and engineering capability.

Bacterial Strain Generation

Vectors were engineered to deliver a mammalian expression reporter plasmid (pCMV-Luc). Three expression parameters associated with LLO were tested: gene dosage, promoter strength, and gene expression regulation (Table 2). An inducible T7 promoter (Studier and Moffatt, J. Mol Biol, 189(1):113-130 (1986)) and a constitutive Tet promoter were selected in this study to accommodate expression variation. Both LLO expression cassettes were either maintained on multi-copy plasmids or integrated into the chromosome of BL21(DE3) at the clp gene location (Parsa, et al., J. Biotechnol. 137(1-4):59-64 (2008)).

High-Throughput Gene Delivery Screen

To expedite the combinatorial screening process, a high-throughput assay was developed to investigate the surface addition of 92 structurally-diverse PBAEs (Table 1). The screen was segmented into three sequential stages of selection. First, all 92 PBAEs were added in three concentrations (0.1, 1.0, and 10 mg/mL) to YWT7-hly/pCMV-Luc (Table 2, strain 1 (S1); chosen initially because of greater relative gene delivery when compared to strains 2-5 (Parsa, et al., J. Biotechnol. 2008, Parsa, et al., Pharm. Res, 2008.)). Upon co-incubation at 22° C. for 15 minutes, hybrid devices were added to 96-well-plated RAW264.7 cells at a 10:1 multiplicity of infection (“MOI,” i.e., ratio of hybrid devices to macrophages).

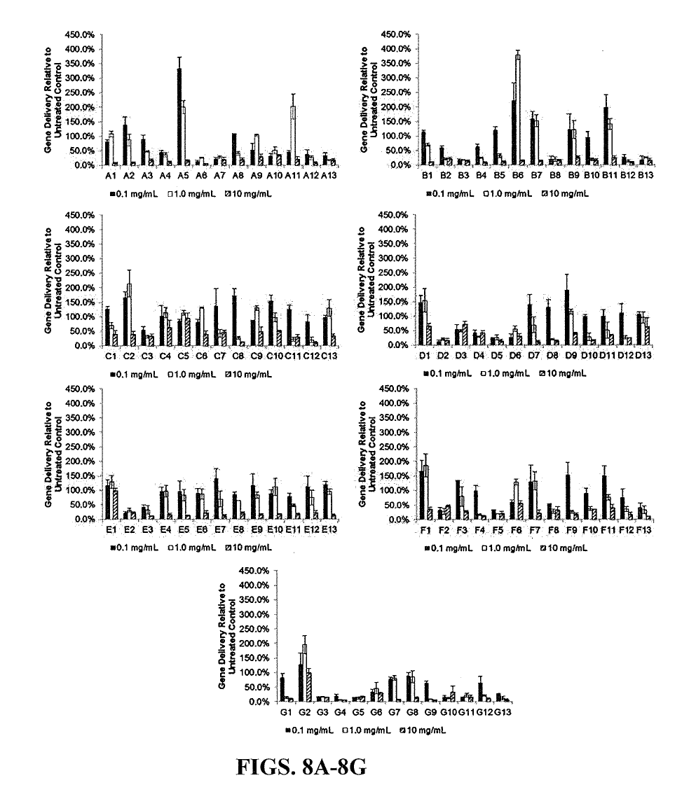

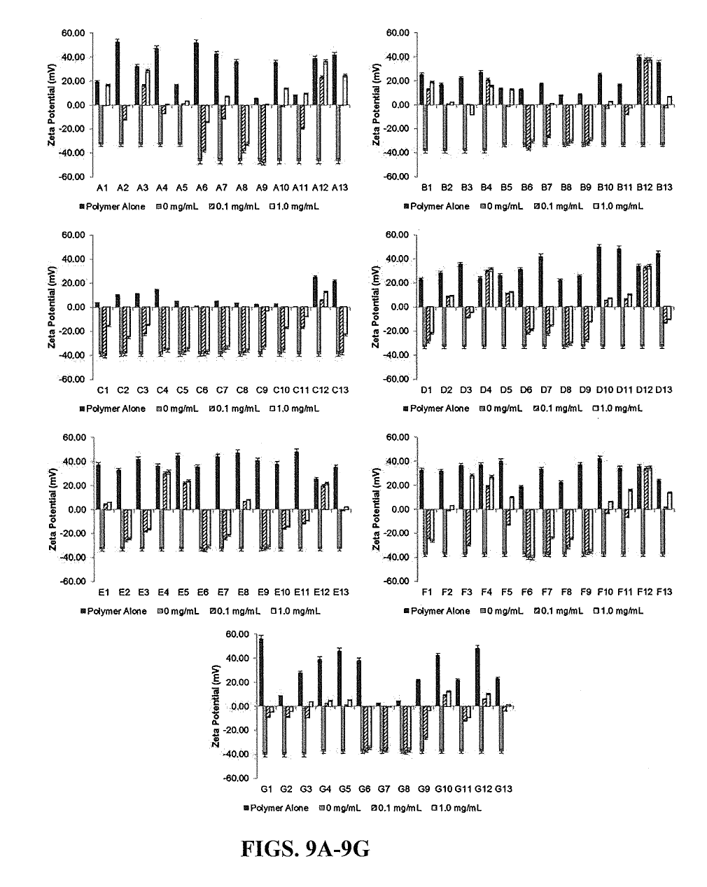

Gene delivery was evaluated by measuring the amount of luciferase expression, standardizing by total protein content, and comparing to untreated bacterial vectors. Numerous improvements in gene delivery beyond bactofection alone were observed across PBAE types and concentration levels with zeta potential readings providing further support for the surface modification of the bacterial component of the hybrid vector (FIGS. 8-9).

From these results, twenty PBAEs (A5, A11, B6, B9, B11, C2, C5, C9, C10, C13, D1, D7, D9, D13, E1, E7, F1, F7, F11, G2) were selected for further analysis in a secondary screen. The highest occurring monomers were C diacrylates (5/20) and four amines (1, 7, 9, and 11; 3/20 each). Interestingly, monomers 1 and 7 contain two amine groups; whereas, monomers 9 and 11 contain two alcohol groups.

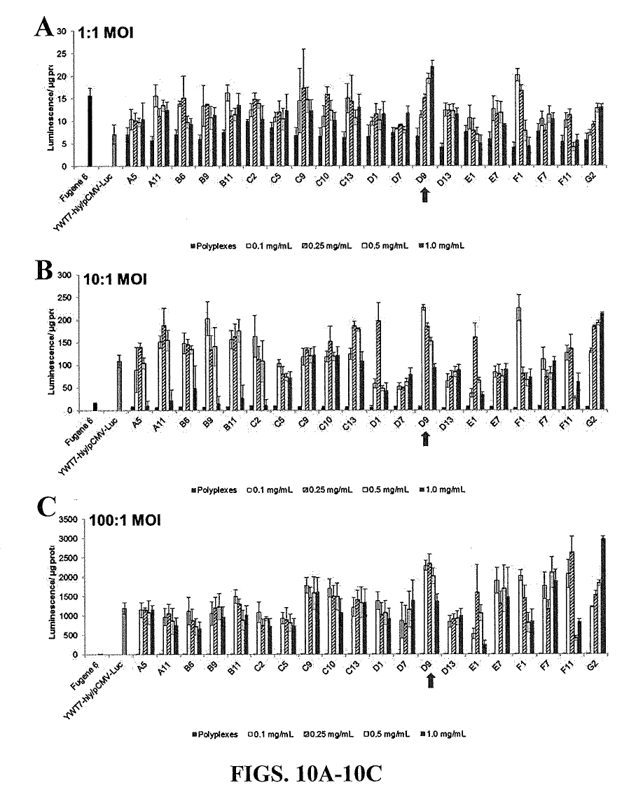

The secondary screen expanded the polymer concentration range (0.1, 0.25, 0.5, and 1.0 mg/mL) and evaluated MOI dependencies. Selection was predicated upon hybrid vectors exceeding gene delivery levels of a positive control (Fugene 6 complexed with 100 ng pCMV-Luc/well) and both the polymer (complexed with 600 ng pCMV-Luc/well) and bacterial vectors in isolation. Hybrid device gene delivery efficacy was influenced by MOI, and within each MOI, different polymer concentration trends were observed (FIGS. 10A-10C). Specifically, at the lowest MOI (1:1), most hybrid devices (14/20) demonstrated positively-correlated concentration-dependent increases in gene delivery. Each of these surpassed the bacterial control but, with the exception of D9, failed to meet or surpass the Fugene 6 control, possibly because of the low amount of pDNA delivered at this MOI. Results are presented in Table 3.

TABLE 3
Efficacy of gene delivery by hybrid vectors including CP D9

| | | | | | | | Gene | Delivery |
|---|---|---|---|---|---|---|---|---|
| | | D9 | Vector | Number of | | Total | Delivery (GD) | Efficiency |
| | | Concentration | Size | Vectors per | pDNA/vector | pDNA | (Luminescence/ | (GD/total |
| Vector | MOI | (mg/mL) | (nm) | Well | (ng) | (ng) | μg protein) | ng pDNA) |
| Strain | 1:1 | 0 | ~2,000 | 30,000 | 2.7E−07^{b} | 0.008 | 7 | 870 |
| 1:D9 | | 0.1 | | | | | 11 | 1,400 |
| Hybrid | | 0.25 | | | | | 15 | 1,880 |
| | | 0.5 | | | | | 19 | 2,410 |
| | | 1 | | | | | 22 | 2,740 |
| | 10:1 | 0 | ~2,000 | 300,000 | 2.7E−07^{b} | 0.08 | 110 | 1,310 |
| | | 0.1 | | | | | 230 | 2,830 |
| | | 0.25 | | | | | 190 | 2,310 |
| | | 0.5 | | | | | 150 | 1,900 |
| | | 1 | | | | | 95 | 1,180 |
| | 100:1 | 0 | ~2,000 | 3,000,000 | 2.7E−07^{b} | 0.8 | 1,190 | 1,480 |
| | | 0.1 | | | | | 2,290 | 2,850 |
| | | 0.25 | | | | | 2,350 | 2,920 |
| | | 0.5 | | | | | 2,020 | 2,500 |
| | | 1 | | | | | 1,380 | 1,720 |
| Fugene | N/A | N/A | N/A | N/A | N/A | 100 | 16 | 0.16 |
| 6 | | | | | | | | |
| D9 | N/A | 900 | 150 | 8.3E+09^{a} | 7.2E−08 | 600 | 7 | 0.01 |
| Polyplex | | | | | | | | |

^{a}Polyplex number calculated by dividing the total mass of D9 added per well at a 30:1 polymer-to-pDNA ratio by an estimated mass of one polyplex. Individual polyplex mass was estimated using the measured effective diameter to calculate volume and a density of 1.22 g/mL (Vauthier).
^{b}The amount of bacterial-maintained pDNA was estimated using a pBR313 origin, which is regulated at 20-40 copies/bacterium, and the molecular weight of the plasmid (estimated as plasmid bp × 650 [Da per bp]).

At a 1:1 MOI, there were 3×10⁴ hybrid vectors/well, and each bacterial-maintained pCMV-Luc plasmid (regulated by the pBR313 origin) was held at 20-40 copies/bacterium (Bolivar), resulting in a final pDNA delivery concentration of 0.008 ng/well (Table 3). Taking the amount of pDNA into account, a new delivery efficiency metric [(luminescence/μg protein)/total ng pDNA] (Parsa, et al., Pharm. Res. 25:1202-1208 (2008)) was utilized to allow better comparison between vectors. Across the three MOIs and PBAE concentrations, the hybrid vector containing polymer D9 was the only vector to consistently exceed bacterial and synthetic controls. Interestingly, substantially different D9 concentration trends were observed across the three MOIs. At the lowest MOI (1:1), hybrid vectors demonstrated a positive concentration-dependent correlation with respect to both gene delivery and delivery efficiency.

However, at 10:1 and 100:1 MOIs, optimal gene delivery and delivery efficiency were observed at the lower D9 concentrations of 0.1 and 0.25 mg/mL, respectively, and gene delivery and delivery efficiency were reduced with continued D9 addition. To further assess gene delivery metrics, additional scoring criteria were introduced to the vectors of Table 3. In particular, population-based delivery efficiency was analyzed by introducing a separate enhanced green fluorescent protein (EGFP) plasmid to the S1 strain (Table 3) and monitoring percent GFP positive cells. When combined with APC viability, a more comprehensive delivery efficiency metric, termed “Total Gene Delivery Performance” (TGDP), emerged as the heading of the last column of Table 3. Using TGDP, it is clear that the hybrid vector outperforms an expanded set of synthetic controls which included seven commercially available transfection agents in addition to D9 and mannosylated D9.

YWT7-Hly Escherichia coli Strains are Optimal for Gene Delivery

Following the identification of an optimal PBAE, (i.e., D9; Acrylate-terminated poly(neopentyl glycol diacrylate-co-2-amino-1,3-propanediol), bacterial strain dependencies were investigated through an expanded study that included five E. coli strains expressing different levels of LLO (Table 2).

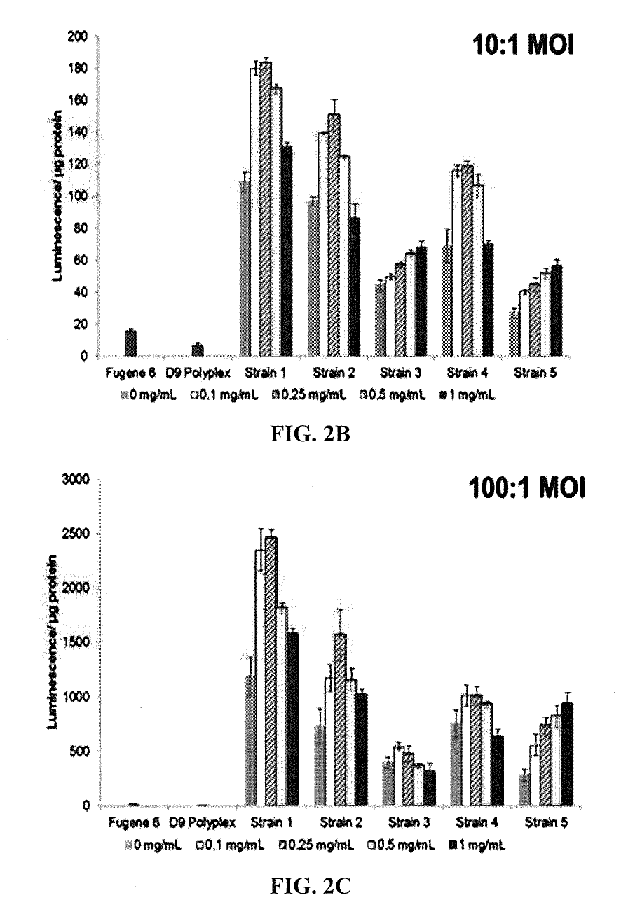

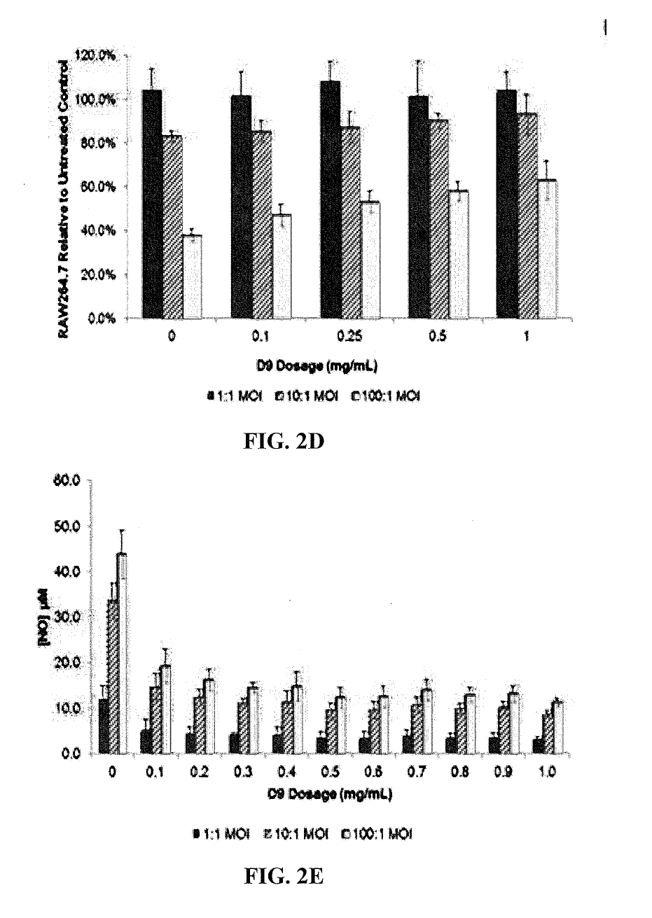

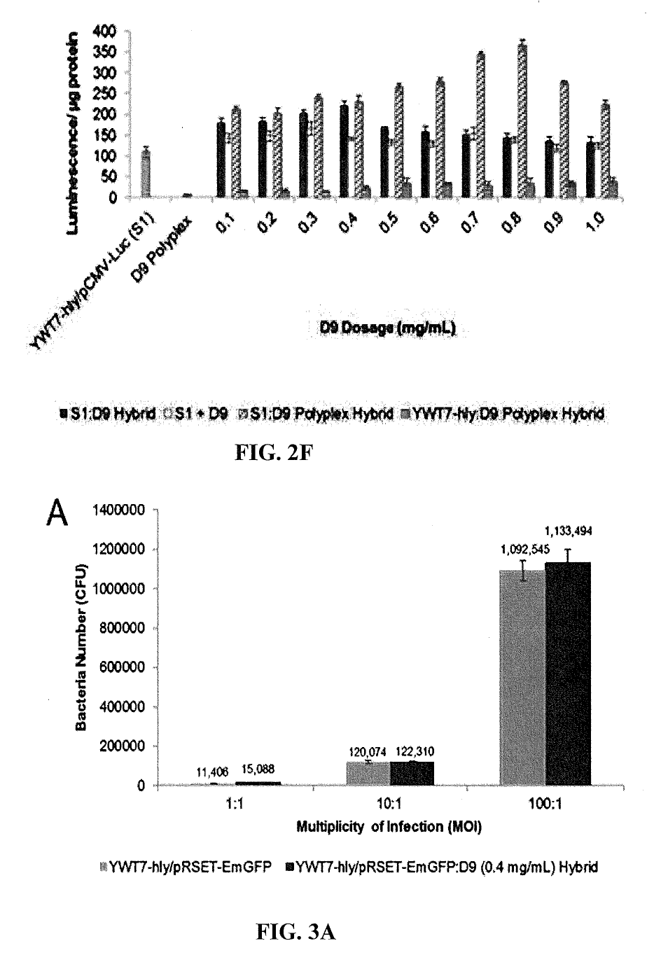

Gene delivery was correlated with MOI, polymer concentration, and LLO expression (FIG. 2A). At 1:1 MOI, gene delivery was positively correlated with D9 concentration regardless of LLO expression. At higher MOIs, hybrid vectors demonstrated the previously observed negatively-correlated gene delivery trend with respect to increasing D9 concentrations with exception to strain 5. Strain 1 (YWT7-hly/pCMV-Luc) chromosome resulted in the most efficacious hybrid vector; however, strain 5, which does not express LLO, demonstrated the largest improvements in gene delivery regardless of MOI or polymer concentration.

Surface addition of D9 to strain 5 presumably compensated for the lack of LLO during vector-mediated phagosomal release. This hypothesis is supported by the lack of significant improvement across MOI and D9 concentrations for strain 3 (3BL21(DE3)/pCMV-Luc/pET29-hly). In this case, relatively high levels of LLO within strain 3 likely precluded synergy with D9 addition suggesting that an optimum exists between the levels of LLO and proton sponge-mediating reagents provided by the hybrid vector.

### Example 2: YWT7-Hly Escherichia coli Strains Including CP D9 are Non-Toxic and Enhance Gene Delivery to APC

### Methods

MTT Assay and NO Production

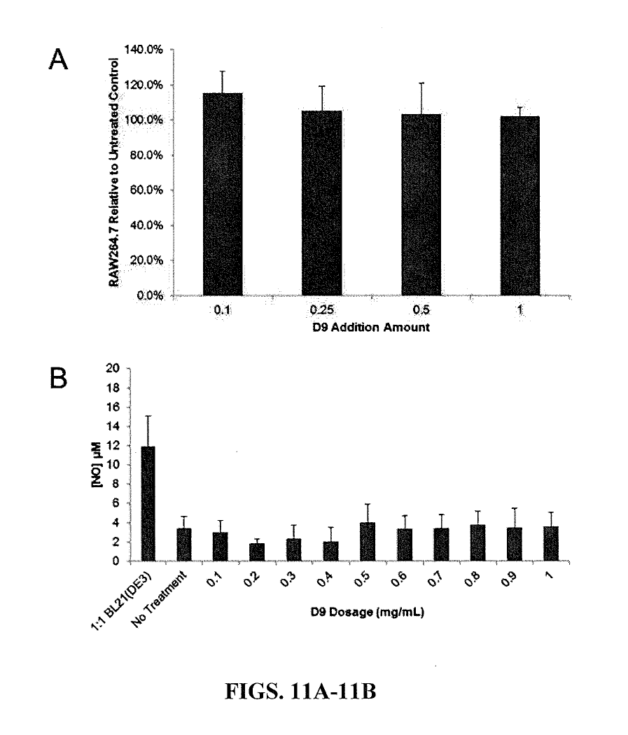

Cytotoxicity resulting from hybrid vectors was determined by the MTT colorimetric assay. RAW264.7 cells were seeded and transfected as described above. Following a 24 h incubation after vector addition, cells were assayed with MTT solution (5 mg/mL), added at 10% v/v, for 3 h at 37° C. with 5% CO₂. Medium plus MTT solution was then aspirated and replaced by DMSO to dissolve the formazan reaction products. Following agitated incubation (using a rotating shaker) for 1 h, the formazan solution was analyzed using a microplate reader at 570 nm with 630 nm serving as the reference wavelength. Results are presented as a percentage of untreated cells (100% viability) (See FIGS. 11A-11B). NO production was measured using a Griess reagent kit (Promega, Madison, Wis.) according to the manufacturer's instructions.

Growth Inhibition

Growth studies were conducted by starting 1% (v/v) liquid cultures from overnight starter cultures and incubating with appropriate selection at 37° C. with shaking at 250 rpm. Samples were measured at OD600 every half hour. 5 ml culture was dissolved in 50 mL LB and cultured to an OD600 value of 0.4, followed by induction with various concentrations of IPTG.

Hybrid Uptake/Load Studies

Uptake and load studies of bacterial and hybrid vectors were evaluated using a high-throughput colorimetric assay. Bacterial and hybrid devices were prepared using the YWT7-hly/pRSET-EmGFP strain.

Macrophage seeding and transfection procedures were conducted as described above except that instead of adding gentamycin-containing media, the transfection solution was aspirated using a multichannel and connected to a vacuum line and replaced by 50 μL of assay buffer (25 μg/mL of trypan blue and 10 μg/mL of polymyxin B). Following a 5 minute incubation period, the assay buffer was aspirated using a multi-channel wand connected to a vacuum line and replaced with PBS. Cells were then analyzed using a plate reader at 487 nm excitation and 509 nm emission compared to a standard curve.

### Results

APC Characterization

For eventual translation, the hybrid device must possess a safe cytotoxicity profile. Thus, S1:D9 hybrids were preliminarily examined for their cytotoxicity at four D9 concentrations (0.1, 0.25, 0.5, and 1.0 mg/mL) and three MOIs (1:1, 10:1, 100:1) (FIG. 2D).

The viability after treatment at 1:1 MOI resulted in no obvious cytotoxicity at all concentrations. Conversely, hybrid devices demonstrated moderate and significant cytotoxicity at 10:1 and 100:1 MOIs, respectively. Resulting cytotoxicity was bacterial-derived as D9 alone did not exhibit toxicity at any concentration (FIG. 11A).

Interestingly, cytotoxicity decreased as D9 concentration increased. Without being bound by any theory, this suggests that CP addition may attenuate bacterial vectors. Similarly, a Griess reagent assay was used to assess macrophage activation via lipopolysaccharide (LPS)-mediated nitric oxide (NO) production (FIG. 2E). NO production levels decreased in a concentration-responsive manner. These data suggest that D9 shields the LPS moieties of the bacterial surface or inhibits intracellular NO formation. Use of D9 alone did not stimulate NO production (FIG. 11B).

Hybrid Vector Characterization

Further insight into APC gene delivery, NO production, and cytotoxicity can be gained from analyzing the hybrid vector design and basic biophysical properties. The influence of each individual component comprising the hybrid device was investigated by the preparation of three types of hybrid vector variants for gene delivery (FIG. 12).

First, S1 was mixed with D9 in RPMI without a dedicated complexation step, resulting in a sample termed S1+D9. Alternatively, pDNA-loaded (600 15 ng/well) polyplexes were prepared and complexed to the surface of YWT7-hly (YWT7-hly:D9 polyplex) and S1 (S1:D9 polyplex).

S1+D9 was designed to investigate co-delivery of two independent vectors (uncomplexed). Conversely, YWT7-hly:D9 polyplex was used to test the ability of LLO-producing bacteria to mediate delivery of a heterologous sequence. S1:D9 polyplex was used to investigate the duality of pDNA-loaded bacterial and CP vectors.

Hybrid vector variants were delivered at a 10:1 MOI with D9 concentration increased from 0.1 to 1.0 mg/mL. The S1:D9 hybrid vector performed optimally with 0.4 mg/mL D9 and was markedly improved when compared to S1+D9 at this concentration level, demonstrating the need for a dedicated surface complexation step (FIG. 2F). In turn, S1:D9 polyplex demonstrated a positive correlation between polymer concentration and gene delivery, with optimal gene delivery occurring at 0.8 mg/mL D9.

Interestingly, only 0.018 mg of D9 is required to fully complex 600 ng of pCMV-Luc (30:1 polymer to pDNA w/w ratio), but increased D9 concentrations mediate elevated gene delivery levels.

The data demonstrate increased gene delivery with excess polymer addition. The initial hybrid vector design utilized the bacterial strain as a means to maintain and transfer pDNA; however, YWT7-hly:D9 polyplex relies solely upon the polyplex component for plasmid maintenance.

In this case, the bacterial strain mediates bulk polyplex transmission and provides a phagosomal escape mechanism via LLO expression. The resulting gene delivery demonstrated concentration dependence upon D9 addition (FIG. 2F), and improvements relative to the D9 polyplex alone further support the combined benefits of the hybrid vector.

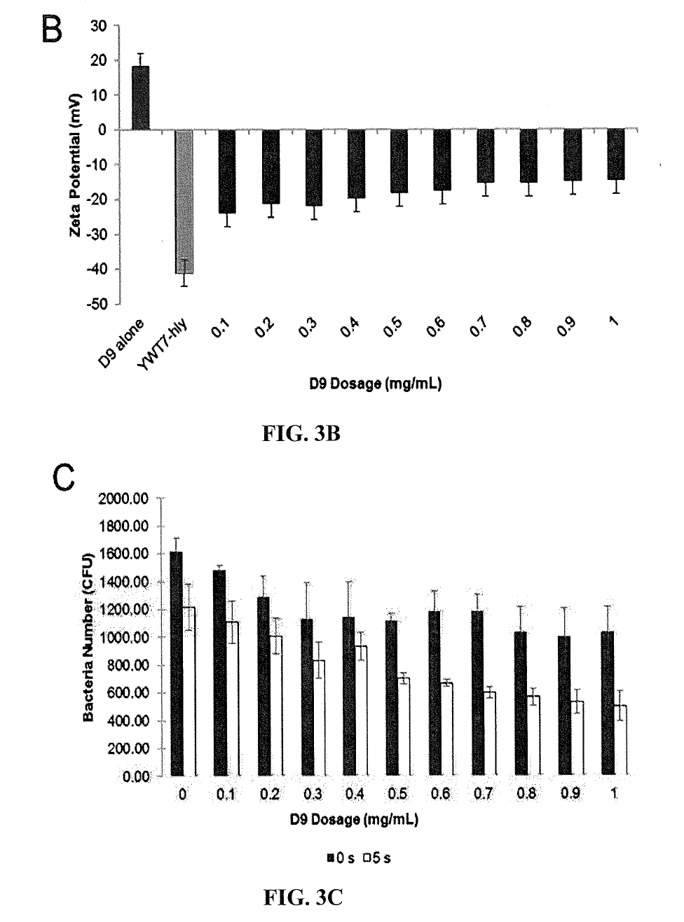

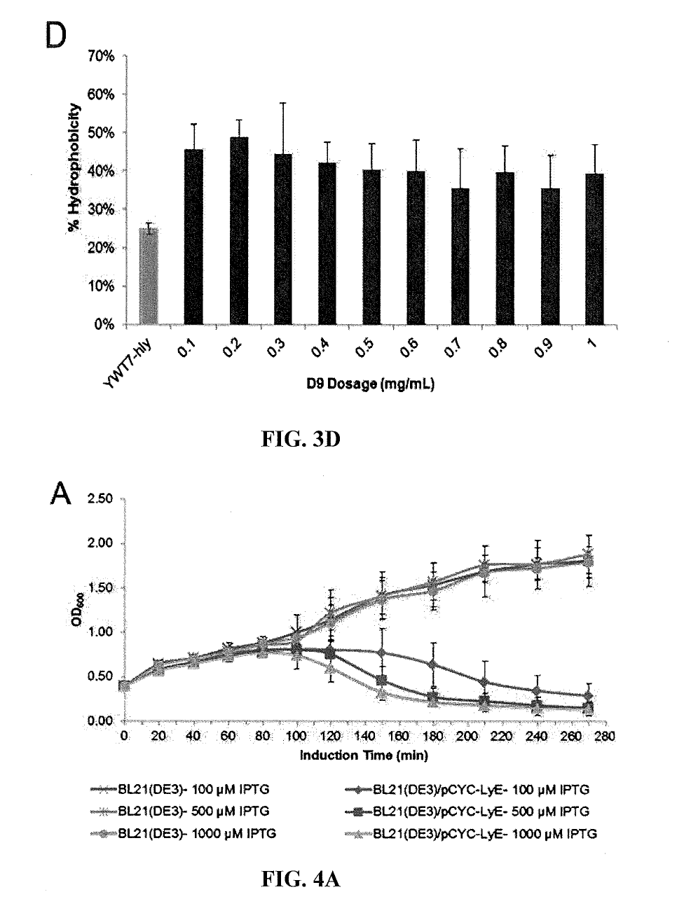

In the associated gene delivery studies (FIGS. 10A-10C), lower MOIs resulted in positively-correlated D9 concentration-dependent increases in gene delivery. However the reason for this was unknown. Thus, a high-throughput assay was developed to investigate the influence of D9 surface addition upon cellular uptake (FIG. 3A). At the 1:1 MOI, the hybrid vector resulted in ˜50% uptake of the total seeded vectors compared to ˜30% for the bacterial control. The increase in uptake helps explain the positive correlation between gene delivery and D9 addition at the lowest MOI. In contrast, at higher MOIs, vectors saturate the APCs (˜40% and ˜30% uptake for 10:1 and 100:1 MOIs, respectively) and thus additional uptake mechanisms become insignificant.

### Example 3: Cationic Polymers Beneficially Attenuate the Bacterial Core

### Methods

Induced bacterial culture and hybrid vector samples (200 μL) were washed and resuspended in PBS, before being sonicated at 20% capacity for 5 seconds using a Branson 450D Sonifier (400 Watts, tapered microtip).

Sonicated samples were plated on LB agar plates and incubated for 24 h prior to counting colony forming units.

### Results

The coating of CPs to the bacterial surface was initiated to increase the surface charge of final hybrid devices (FIG. 3B). The increased charge may aid the electrostatic attraction to mammalian cells and facilitate subsequent uptake.

The surface coating may beneficially attenuate the bacterial core of the hybrid device. To test this possibility and expand upon the reduced cytotoxicity afforded by D9 surface additions (FIG. 2D), hybrid vectors were tested in shear disruption studies conducted through brief exposure to sonication (FIG. 3C).

Without sonication, hybrid vectors demonstrated reduced viability (compared to the bacterial control) but did not exhibit significant differences between D9 concentrations. However, upon sonication, hybrid device viability was reduced in a D9 concentration-dependent manner. Antibiotics like polymyxin B (PLB) promote bacterial membrane destabilization as a result of strong cationic head-groups (Carr, Cardoso). Analogously, the most cationic PBAEs from the 92-polymer library, even when applied at concentration values below 0.1 mg/mL, resulted in significant viability reduction in the context of the hybrid vector sonication shear assay. In some cases, membrane destabilization mediated by highly charged PBAEs may have resulted in hybrid devices too fragile to demonstrate efficacious gene delivery. However, in other cases, the CPs likely contributed to greatly improved APC gene delivery and viability.

### Example 4: Cationic Polymer Coating Density Influences Bacterial Uptake

### Methods

Characterization of Hybrid Devices

Zeta potentials of bacterial, polymer, and hybrid vectors were measured by DLS. To measure surface hydrophobicity of bacteria before and after PBAE additions, samples were analyzed using a modified microbial adhesion to hydrocarbon (MATH) assay (Pack, Lynn). Briefly, bacterial and hybrid vectors were prepared and resuspended in PBS to a final OD600 value of 1.0. One milliliter of bacterial or hybrid vector was added to a clean glass tube in addition to 110 μL of n-hexadecane (10% v/v). Each sample was then vortexed for one minute at setting 10 (Analog Vortex Mixer, Fisher Scientific) and allowed 5 to settle for 15 minutes for phase separation.

Using a clean Pasteur pipet, bacterial/hybrid vector solution was retrieved, taking care to avoid the upper hydrocarbon layer, and transferred to a cuvette for a final OD600 measurement. The percentage change of hydrophobicity is calculated by:

$$Δ Hydro \% = \frac{( {A}_{initial} - {A}_{final} )}{{A}_{initial}}$$

Preparation of Vectors for Scanning Electron Microscopy

Bacterial and hybrid device samples were deposited onto a 0.45-μm-pore-size membrane filter, fixed for 1 h with 2% glutaraldehyde, washed four times, and dehydrated with a graded ethanol series. Following dehydration, 100% HMDS was added, and the sample was allowed to dry uncapped. For washing and dilution of glutaraldehyde, 0.15 M sodium phosphate buffer (pH 7.2) was used. Carbon was sputtered on the samples to avoid charging in the microscope.

### Results

The surfaces of bacterial vectors were modified with CPs possessing significant stretches of hydrophobic domains to affect final hybrid vector polarity. Using a modified microbial adhesion to hydrocarbon (MATH) assay (Geertsemadoornbusch, et al., Journal of Microbiological Methods, 18(1):61-68 (1993); Rosenberg, FEMS Microbiology Letters 262(2):129-134 (2006)), hybrid vectors prepared using D9 demonstrated increased hydrophobicity regardless of polymer concentration, representing a 2-fold increase over the bacterial control (FIG. 3D).

To visually confirm D9 addition, hybrid and bacterial vectors were analyzed by scanning electron microscopy (SEM), revealing concentration-dependent surface modification. Interestingly, synergistic concentration ranges (<1 mg/mL) contained individualized cells with polymeric extensions. Treatment with excess polymer (5 and 10 mg/mL), however, resulted in heavy surface coating and systemic adherence between hybrid vectors. Coalescence of hybrid vectors is a likely cause for the decline in gene delivery at elevated polymer concentrations (FIGS. 8A-8G) due to the inability to phagocytose resulting conglomerates (Doshi and Mitragotri, Plos One 5(3) (2010); Champion, et al., Pharm Res, 25(8):1815-1821(2008)).

### Example 5: Bacteriophage 5 ΦX174 Lysis Gene E Incorporation Enhances Gene Delivery

### Methods

To demonstrate the biological engineering potential of the hybrid device, bacteriophage cDX174 LyE was introduced and conditionally expressed. Production of the resulting endolysin leads to the formation of transmembrane tunnels through the bacterial cellular envelope (Eko, et al., Vaccine 17(13-14):1643-1649(1999); Witte et al., Archives of Microbiology 157(4):381-388 (1992)). These compromised bacterial vectors may experience further membrane destabilization upon lysosomal entrapment, facilitating increased release of LLO and pDNA. Elevated concentrations of LLO may instigate additional lysosomal rupture and increase the effective concentration of cytosolic pDNA for nuclear translocation.

The lethal lysis gene E (LyE) from bacteriophage ΦX174 was amplified from genomic DNA provided by Dr. Ryland Young (Department of Biochemistry & Biophysics, Texas A&M University) using forward primer: AGG GAA TTCG ATG GTA CGC TGG ACT TTG TGG and reverse primer: AGG AAG CTT TCA CTC CTT CCG CAC GTA ATT.

The gel-purified ˜280 bp band was digested with EcoRI and HindIII and ligated into pACYCDuet-1 (Novagen) digested with the same enzymes to generate pCYC-LyE. The vector encodes two multiple cloning sites (MCS) each of which is preceded by a T7 promoter, lac operator and ribosome binding site (rbs). The vector also carries the P15A replicon, lad gene and chloramphenicol resistance gene. The pCYC-LyE construct was screened and confirmed by colony PCR and restriction digest analysis.

### Results

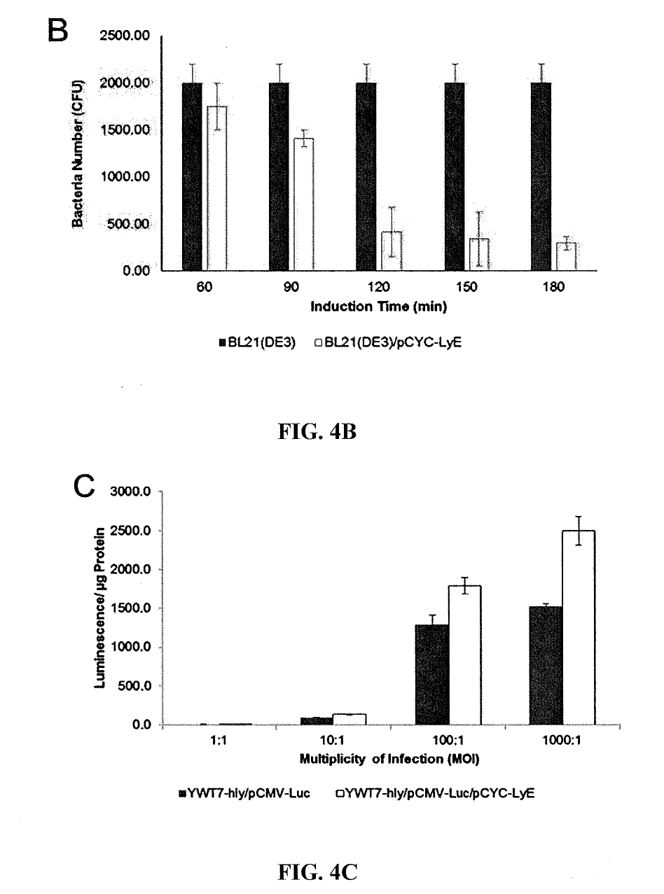

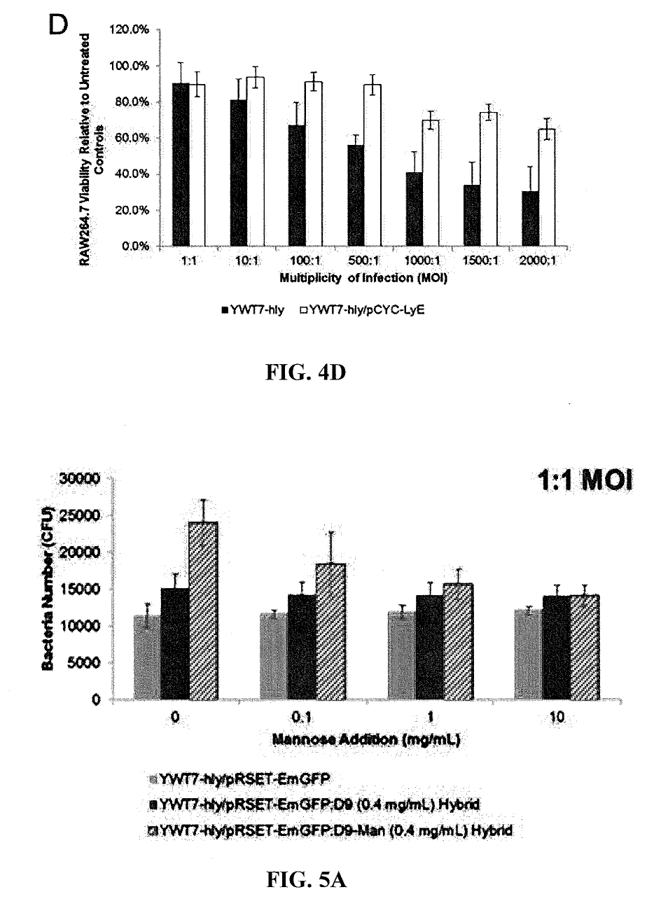

Initially, pCYC-LyE was tested in growth studies at three IPTG induction concentrations (FIG. 4A). Bacterial strains containing LyE demonstrated IPTG concentration dependent lysis. Critical point lysis occurred at 150, 120, and 100 minutes for 100, 500, and 1,000 μM IPTG, respectively. Taking these observations into account, shear studies with 5 second sonication times were performed with 1,000 μM IPTG-induced LyE-containing bacterial strains (FIG. 4B). Noticeable viability reduction occurred after a 90 minute induction period.

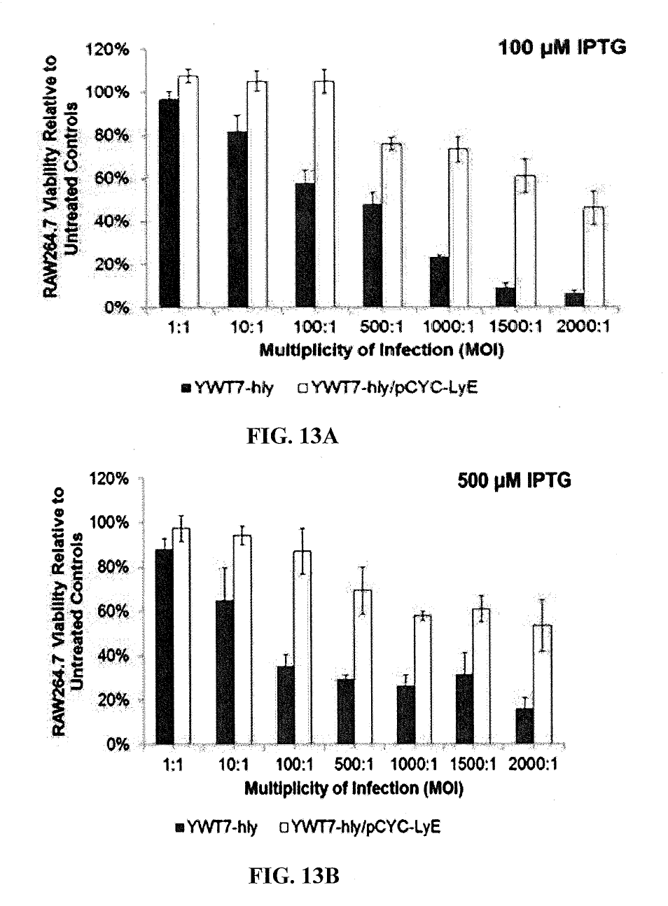

To further extend the properties of the bacterial gene delivery vectors, pCYCLyE was incorporated into the previously best-performing strain, YWT7-hly/pCMV-Luc (S1). Using 1,000 μM IPTG induction for one hour, gene delivery experiments were conducted at 1:1, 10:1, 100:1, and 1,000:1 MOIs (FIG. 4C). LyE-containing S1 demonstrated improved gene delivery in relation to increasing MOI. It could be that membrane destablization resulted in elevated gene delivery due to increased pDNA and protein release coupled to decreased APC cytotoxicity. Thus, to evaluate the impact of LyE upon cytotoxicity, bacterial strains were induced with 100, 500, and 1,000 μM IPTG and transfected at 1:1, 10:1, 100:1, 500:1, 1,000:1, 1,500:1, and 2,000:1 MOIs. At 100 and 500 μM IPTG induction (FIGS. 13A-13B), LyE expression significantly improved cytotoxicity as compared to the bacterial controls. At excessive MOI levels such as 2,000:1, LyE-containing S1 exhibited 46% and 53% viability at 100 and 500 μM IPTG, respectively.

Upon higher induction (1,000 μM IPTG), 2,000:1 MOI viability was further elevated to 65% (FIG. 4D). Using the highest IPTG concentration, gene delivery was roughly doubled with ˜80% viability at 1,000:1 MOI.

### Example 6: Mannosylation of Cationic Polymers Enhances Targeted Delivery of Bacterial Hybrid Devices

Mannose was attached to the terminal end of the optimal PBAE prior to hybrid device foiination, resulting in increased vector uptake and gene delivery.

### Methods

Synthesis of Mannosylated PBAE

To improve upon bacterial uptake and delivery specificity, mannose, an antagonist of CD206 (primarily expressed on APCs) (Daigneault, et al., Plos One 5(1):e8668 (2010); Devey L, et al. Mol Ther 17(1):65-72(2009)), was grafted onto D9 (for description of end modification of PBAE, see generally, Eltoukhy, et al., Biomaterials 33(13):3594-3603(2012); Zugates, et al., Mol Ther 15(7):1306-1312(2007) Sunshine 2011, Zugates 2007 Bioconjug Chem, Eltoukhy, Zugates 2007 Mol Ther). Specifically, D9 was end-capped by D-mannose.

Mannosylated-D9 was synthesized using a three-step procedure (FIGS. 14A-14D). First, acrylate-terminated poly(neopentyl glycol diacrylate-co-2-amino-1,3-propanediol), D9ac, was synthesized without solvent 5 at a 1:1.2 amine (2-amino-1,3-propanediol):diacrylate (neopentyl glycol diacrylate) molar ratio (using 400 mg amine) at 90° C. for 24 h (FIG. 14A).

As a second step, ethylenediamine was reacted in excess with D9ac to amine-cap the terminal ends (FIG. 14B). Specifically, D9ac was dissolved in DMSO at 167 mg/mL and 500 μL reacted with 500 μL of 0.5 M ethylenediamine (in DMSO) at 22° C. for 24 h.

Amine-capped D9 (D9-am) was then reacted with allyl-α-D-mannopyranoside (ADM) at a 1:2 molar ratio in DMSO at 22° C. for 24 h. Allyl-α-D-mannopyranoside was synthesized by dissolving 3 g of D-mannose and 18 mg p-TsCl in allyl alcohol (20 mL) at 90° C. under reflux for 24 h (FIG. 14D).

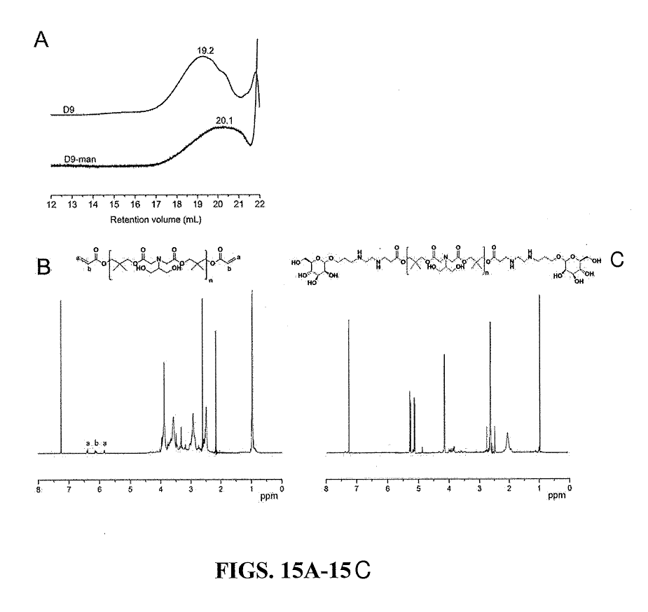

A detailed protocol is set forth in FIGS. 14A-14D. This was accomplished by synthesizing acrylate-capped D9 (D9ac) and further modifying with an end-capping amine (D9am). In parallel, allyl-α-D-mannopyranoside (ADM) was synthesized and reacted with D9am to produce mannose-terminated D9 (D9-Man). Synthesis was confirmed using GPC and NMR (FIGS. 15A-15B).

### Results

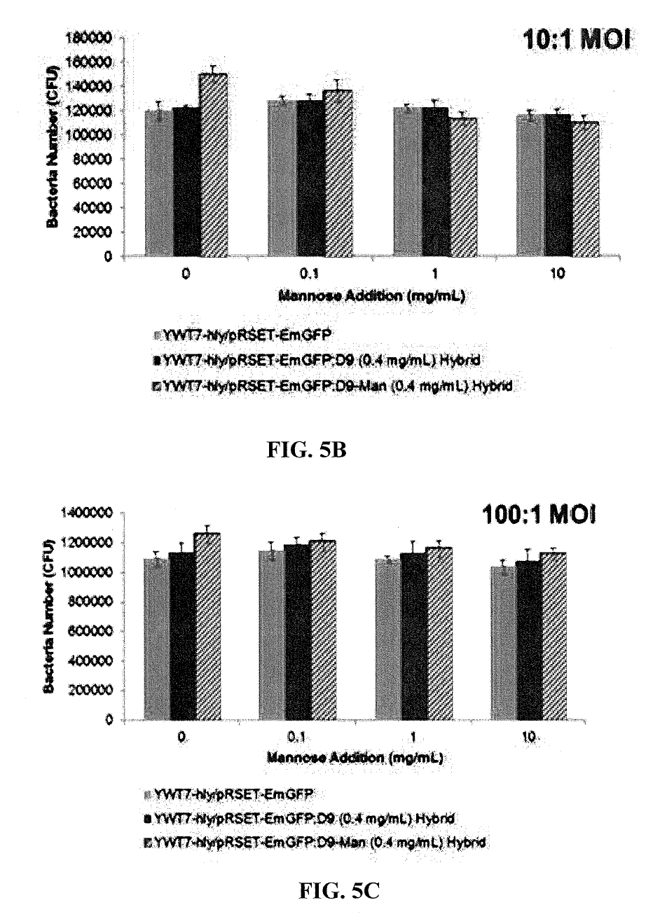

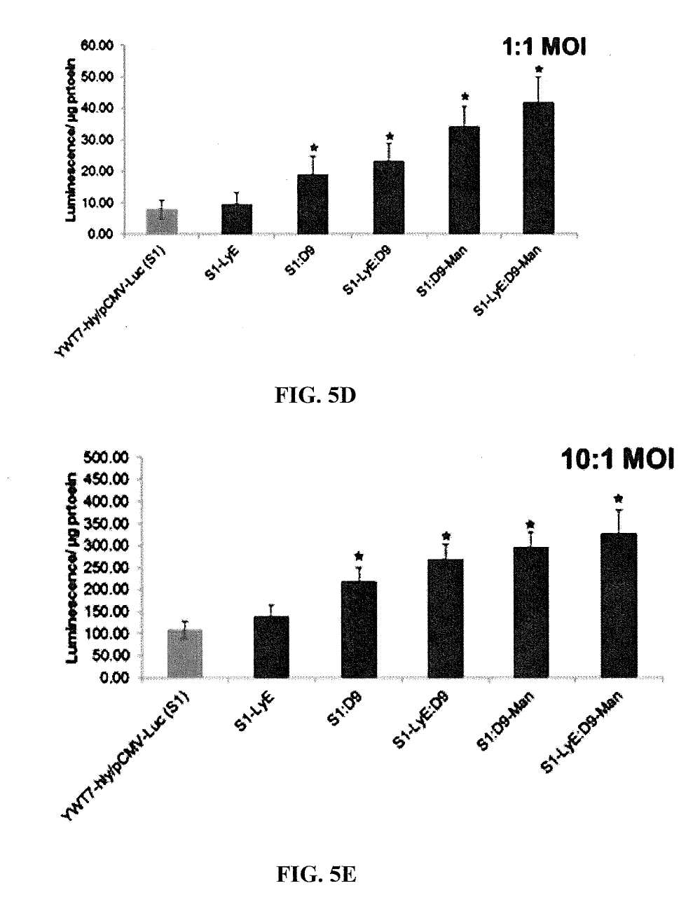

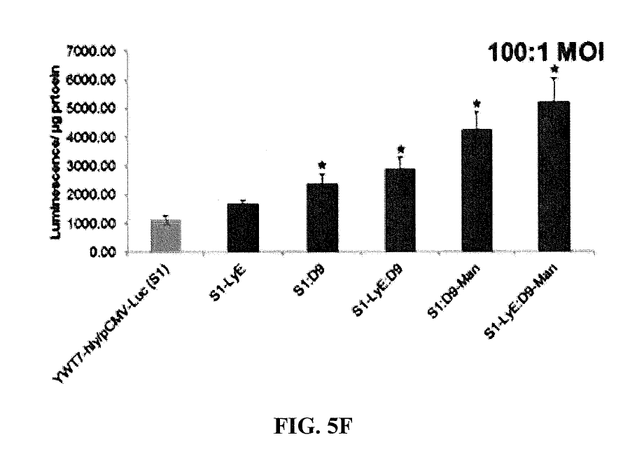

Mannosylated hybrids (0.4 mg/mL) were prepared with a bacterial strain (YWT7-hly/pRSET-EmGFP) expressing an emerald green fluorescent protein (EmGFP) and evaluated for uptake (FIGS. 5A-5C) and gene delivery (FIGS. 5D-5F). Additional D-mannose was included to test competitive inhibition of the CD206 receptor. At 1:1 and 10:1 MOIs, uptake of mannosylated hybrids was significantly higher than either the bacterial or unmannosylated hybrid vectors in the absence of exogenously added mannose. However, upon addition of free mannose, uptake of only the mannosylated hybrid vector decreases proportionately.

At the highest MOI, 100:1, uptake of mannosylated is slightly elevated compared to controls and decreases upon mannose addition.

Finally, hybrid devices were combined with previous LyE strains to test the feasibility of a dual-engineered hybrid vector (FIGS. 5D-5F). Hybrid vectors were prepared and transfected using D9 and D9-Man at 0.4 mg/mL with S1 and S1/pCYC-LyE (S1-LyE). Gene delivery was improved across all MOIs by the separate molecular biology (introduction of LyE) and polymer chemistry (mannosylation of D9) toolsets uniquely afforded by the hybrid vector. The final approach and results clearly indicate the synergistic and engineering potential of the hybrid vector design. Complementary flow cytometry population data was collected for all samples in FIGS. 5D-5F, with the S1-LyE:D9-Man vector showing the best combination of highest % GFP positive cells with no associated APC cellular toxicity.

### Example 7: Polymer Molecular Weight and Mannose Content Influences Uptake and Delivery of Nucleic Acids by Mannosylated Hybrid Bacterial Vectors

### Methods

Mannosylated Poly(Beta-Amino Esters) Synthesis

Polymers were synthesized using a previously developed three-step reaction (Jones, et al. Proc Natl Acad Sci USA.; 111:12360-5(2014); Jones, et al. Biomaterials. 37:333-44 (2015)). Briefly, diacrylate-capped polymers were first synthesized in DMSO at various diacrylate/amine molar ratios (D:A ratio) for 5 days at 60° C. The initial polymer library was synthesized using a 1.2 D:A ratio (Table 4); whereas, the expanded polymer set utilized a wider range (Table 5). Synthesis of the polymer library was conducted according to the following 3-step scheme (scheme III):

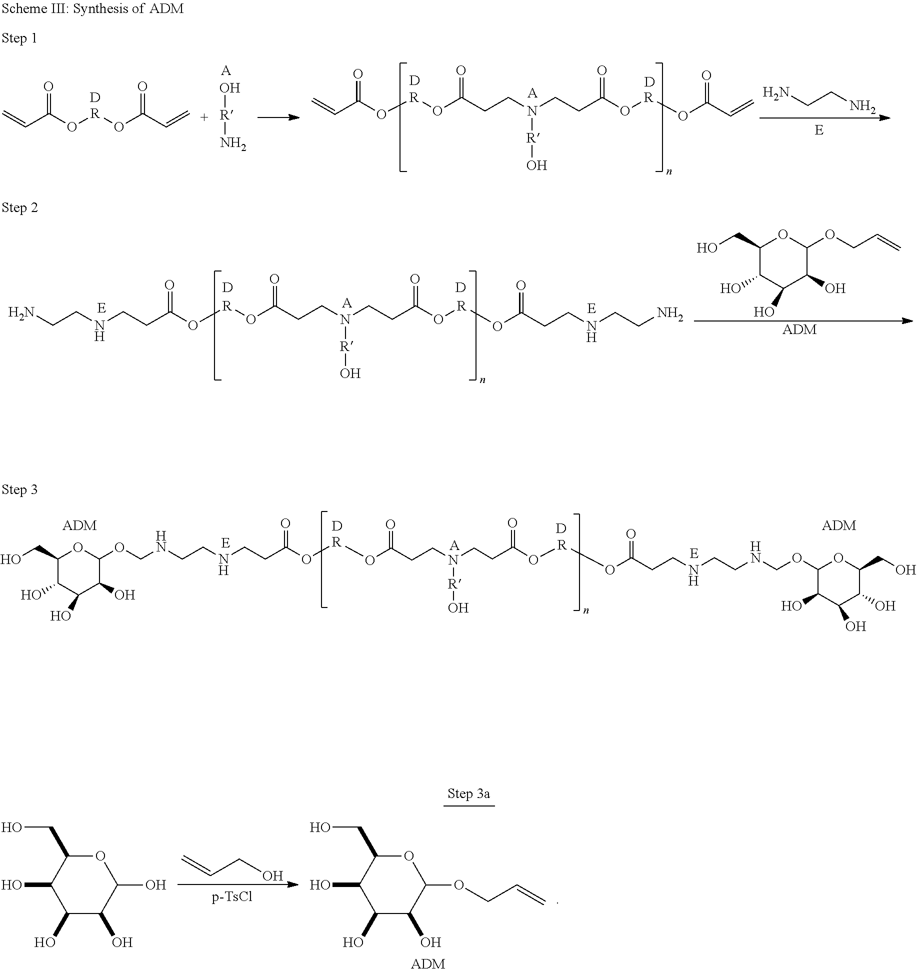

Synthesis was carried out according to the above using the monomers in Formula IV:

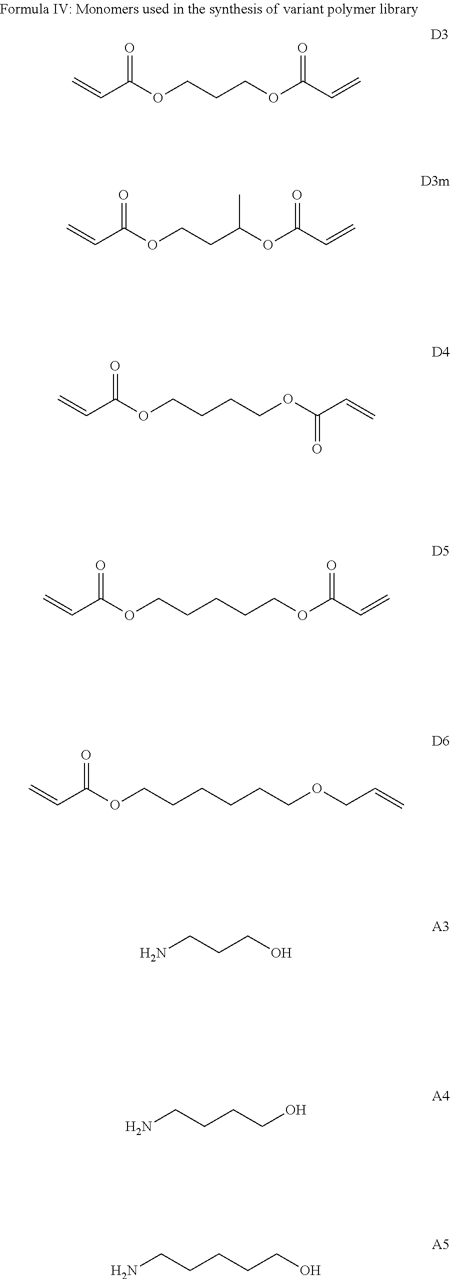

Variance of molar ratios in the expanded polymer set permitted tunable control of the base polymer molecular weight (Table 5).

All acrylate-terminated polymers were then reacted with excess ethylenediamine to amine-cap the terminal ends. Specifically, acrylate-terminated polymers were dissolved in DMSO at 167 mg/mL and reacted with 5 M ethylenediamine (in DMSO) at room temperature for 24 h. Amine-capped polymers were purified by dialysis followed by evaporation under vacuum. Dialysis procedures were conducted against acetone using molecular porous membrane tubing (Spectra/Por Dialysis Membrane, Spectrum Laboratories Inc.) with an approximate molecular weight cut off at 3,500 Da. As a last step, amine-capped PBAEs were then reacted with allyl-α-D-mannopyranoside (ADM) at a 1:2 molar ratio in DMSO at 90° C. for 24 h and then purified via dialysis. Structure and purity of polymers were confirmed using ¹H NMR spectroscopy (Jones, et al. Biomaterials.37:333-344 (2015)). ADM was synthesized by dissolving 3 g of D-(+)-mannose and 18 mg p-TsCl in allyl alcohol (20 mL) at 90° C. under reflux for 24 h. The reaction solution was then concentrated by vacuum distillation at 35° C.

TABLE 4
Polymer Synthesis and Characterization Summary of
Base Polymers

| | | | | | Zeta |
|---|---|---|---|---|---|
| | | | | Zeta Potential | Potential |
| | | Mₙ^{GPC} | M_{w}^{GP} | (mV) PBS (pH | (mV)NaOAc |
| Polymer | PDI^{GPC} | (Da) | (Da) | 7.4) | (pH 5.15) |
| D3mA4 | 1342 | 7,839 | 10,520 | −19.4 | 28.8 |
| D3mA4-Man | 1.696 | 6,996 | 11,863 | −6.4 | 20.1 |
| D3A5 | 1.418 | 8,214 | 11,643 | −18.6 | 37.5 |
| D3A5-Man | 1.640 | 5,083 | 8,337 | −4.1 | 31.7 |
| D3mA5 | 1.206 | 8,848 | 10,667 | −19.9 | 33.1 |
| D3mA5-Man | 1.574 | 8,128 | 12,794 | −12.6 | 28.7 |
| D4A3 | 1.405 | 9,617 | 13,511 | −12.5 | 34.5 |
| D4A3-Man | 1.680 | 4,403 | 7,397 | −9.9 | 28.6 |
| D4A4 | 1.629 | 8,571 | 13,961 | −19.5 | 25.9 |
| D4A4-Man | 1.377 | 4,967 | 6,839 | −12.4 | 20.6 |
| D4A5 | 1.453 | 8,520 | 12,380 | −16.7 | 28.5 |
| D4A5-Man | 1.542 | 4,762 | 7,345 | −11.2 | 34.2 |
| D5A4 | 1.377 | 8,927 | 12,294 | −12.7 | 21.2 |
| D5A4-Man | 1.622 | 8,701 | 14,109 | −8.4 | 26.3 |
| D5A5 | 1.319 | 9,577 | 12,632 | −26.0 | 31.1 |
| D5A5-Man | 1.501 | 7,040 | 10,567 | −15.5 | 25.5 |
| D6A5 | 1.472 | 8,286 | 12,198 | −21.8 | 34.2 |
| D6A5-Man | 1.664 | 7,517 | 12,508 | −18.4 | 33.4 |

TABLE 5
Synthesis and characterization summary of
molecular weight variants of D4A4-Man

| | | | | | | Zeta |
|---|---|---|---|---|---|---|
| Poly- | | | | | Zeta | Potential |
| mer | D4:A4 | | | | Potential | (mV) |
| Desig- | Feed | | Mₙ^{GPC} | M_{w}^{GPC} | (mV) PBS | NaOAc |
| nation | Ratio | PDI^{GPC} | (Da) | (Da) | (pH 7.4) | (pH 5.15) |
| P1 | 1.025 | 2.477 | 13,623 | 33,745 | −8.6 | 32.6 |
| P2 | 1.0375 | 1.641 | 13,425 | 22,031 | −5.3 | 34.0 |
| P3 | 1.05 | 1.536 | 11,961 | 18,372 | −6.2 | 33.6 |
| P4 | 1.0625 | 1.370 | 11,848 | 16,232 | −8.3 | 32.4 |
| P5 | 1.075 | 1.629 | 10,474 | 17,063 | −7.7 | 32.7 |
| P6 | 1.1 | 1.642 | 10,130 | 16,633 | −8.8 | 34.6 |
| P7 | 1.125 | 1.833 | 7,902 | 14,484 | −8.0 | 31.9 |
| P8 | 1.15 | 1.468 | 7,312 | 10,734 | −11.1 | 27.5 |
| P9 | 1.175 | 1.699 | 6,589 | 11,195 | −12.1 | 28.6 |
| P10 | 1.2 | 1.549 | 5,966 | 9,241 | −11.2 | 24.9 |
| P11 | 1.225 | 1.590 | 5,510 | 8,761 | −11.4 | 26.4 |
| P12 | 1.25 | 1.600 | 5,112 | 8,180 | −12.7 | 24.0 |
| P13 | 1.275 | 1.638 | 4,673 | 7,654 | −12.9 | 20.9 |
| P14 | 1.3 | 1.615 | 4,479 | 7,234 | −11.2 | 21.4 |

Gene Delivery Inhibition Studies

To determine if transfection was affected by the presence of free mannose and physiological levels of serum, hybrid vectors were prepared as described above. Vectors were then transfected as before using RAW264.7 cells with the following alterations. First, 30 minutes prior to transfection, media was replaced with 50 μL growth medium containing 1,000 μM of free mannose and/or 50% v/v FBS. Transfection occurred as before except initial media was not replaced, but rather, hybrid vectors were added to yield a 100 μL volume (and 150 μL after incubation and gentamicin-containing medium addition).

### Results

Polymer Synthesis and Characterization

The synthesis of the PBAE-Man polymers proceeds via a three-part conjugate (Michael) addition of amines to acrylate groups. Utilizing the high-throughput polymer synthesis methodology, 18 PBAE-Man polymers were produced from a diverse set of monomers Sunshine, et al., Plos One. 7:e37543(2012); Sunshine, et al., Mol Pharm. 9:3375-3383(2012); Anderson, et al., Mol Ther. 11:426-34 (2005); Anderson, et al., Angewandte Chemie. 42:3153-8 (2003)). The library was synthesized on a 1- to 2-g scale with increased monomer concentrations to provide greater control of stoichiometry, increase polymer molecular weight and end-group termination, and reduce potential intramolecular cyclization.

D4A4-Man was identified as the optimal polymer. The amine to diacrylate monomer (D4:A4) ratio was systematically varied to produce a second library of stratified molecular weight polymers with the same chemical background.

Each respective polymer structure and purity (from both polymer libraries) was confirmed using ¹H NMR. GPC and DLS were used to measure molecular weight, polydispersity index (PDI), and zeta potential (in two buffers) of the polymers (Tables 4 and 5). The generated polymer libraries spanned various chemical backgrounds and molecular weights ranging from 6.8 to 33.7 kDa (weighted MW) while demonstrating broad PDI values (˜1.2-2.5) characteristic of PBAEs. Following structure confirmation, each polymer library was evaluated for charge densities (zeta potential) in two physiologically-mimicking buffers. Both polymer libraries possessed negative charges in neutral buffer and cationic charges in acidic conditions. Furthermore, the zeta potential values of the D4A4 polymer library increased proportionally with increases in molecular weight regardless of buffer (Table 5).

Hybrid Vector Formation and Characterization

A single E. coli strain was selected as the bacterial vector based upon previous optimization studies (Jones, et al. Proc Natl Acad Sci USA. 111:12360-5 (2014); Jones, et al., Mol Pharm.10:4301-4308 (2013); Parsa, et al., J Biotechnol. 137:59-64 (2008); Parsa, et al., Pharm Res. 25:1202-8 (2008)). This particular bacterial strain was engineered to deliver a mammalian expression reporter plasmid (pCMV-Luc) with assistance of a chromosomal-located listeriolysin O (LLO) expression cassette driven by an inducible T7 promoter (Studier and Moffatt, J. Mol. Biol, 189:113-30 (1986)) at the clpP location (Parsa, et al., J Biotechnol. 137:59-64 (2008)).

Hybrid bio-synthetic vectors are formed through electrostatically-driven interactions between positively charged polymers (derived from protonated amines) and the negatively charged outer membrane of E. coli enabling a simple mixing methodology and the potential for rapid vector formulation scalability. Surface deposition of cationic polymers to the bacterial core resulted in a beneficial attenuation phenomenon that was driven by a proposed membrane “integrative” model. Specifically, polymer molecules are believed to first adsorb to the surface of bacteria prior to mediating mild disruptions of the outer and inner Gram-negative leaflet membranes through diffusive mechanisms. Thus, bacterial membrane destabilizations has been associated with improved gene delivery outcomes by facilitating the controlled release of internal protein and DNA cargo (Jones, et al., Mol Pharm, 10:4301-4308 (2013); Chung, et al., Mol Pharm. 2015; DOI: 10.1021/acs.molphamiaceut.5b00172).

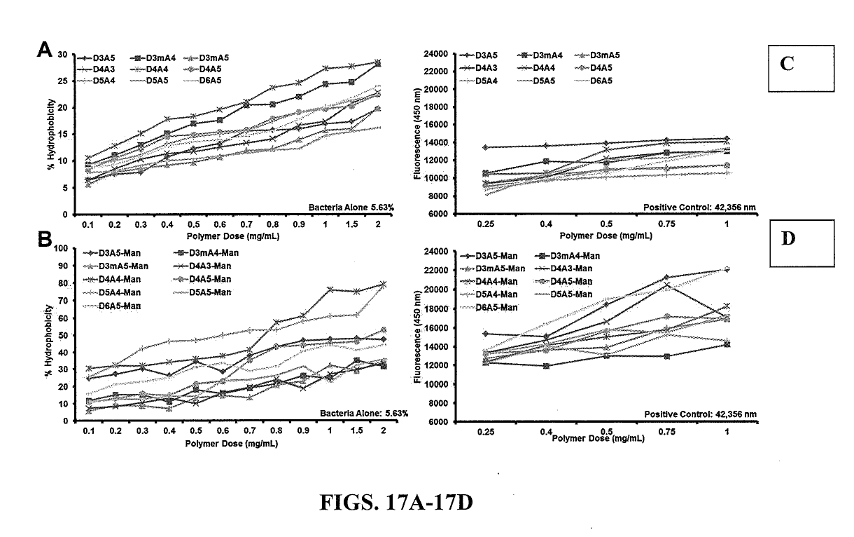

Accordingly, the degree of polymer-mediated surface coverage was evaluated for bacterial membrane hydrophobicity increases and destabilization (FIGS. 17A-17D). Surface hydrophobicity was positively correlated with polymer dose and mannosylation (FIGS. 17A-17B). Mannosylation as a whole facilitated increased surface coverage. This may be the result of a shielding effect provided by mannose molecules that reduces charge-charge repulsion of discrete polymer molecules as they attach to the bacterial surface.

Following the “integrative” model, after surface deposition polymers were evaluated for their respective membrane destabilization potential via a novel colorimetric assay (as compared to our previous adsorption-based assessment of released protein and DNA). Specifically, over-expression of BFP in E. coli prior to polymer addition provides a colorimetric molecule that will diffuse through polymer-mediated membrane disruptions. Membrane destabilization increases with mannosylation (FIG. 17B; FIG. 17D) independent of chemical background. In addition, for both polymer libraries, the polymer possessing the highest zeta potential (D3A5-Man) mediated the highest membrane destabilization. However, none of the polymers were able to facilitate complete disruption as demonstrated by a known membrane-active antibiotic (polymyxin B).

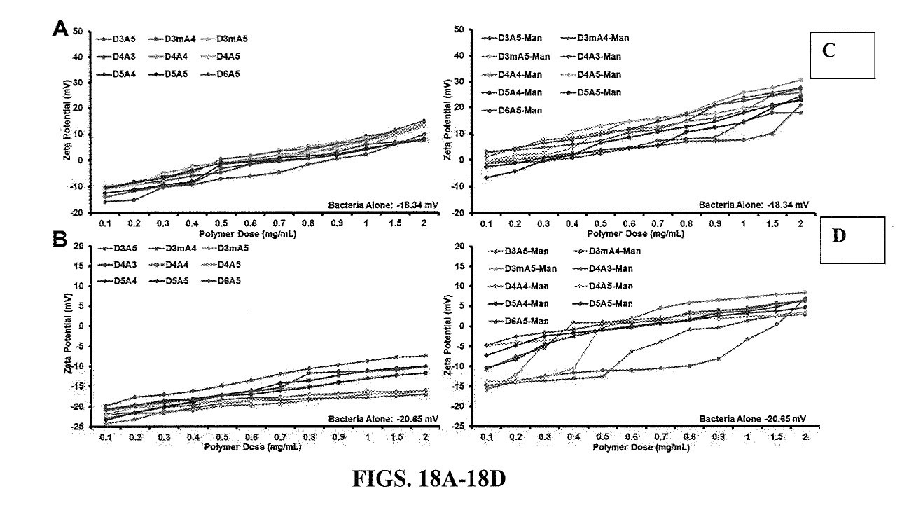

In addition to the bacterial effects upon polymer addition, the vector as a whole was evaluated for the macroscopic properties that are displayed to the environment. Namely, the zeta potential of hybrid vectors was evaluated at various polymers doses in two physiologically relevant pHs (FIGS. 18A-18D). Each pH represents either the extracellular environment (FIG. 18B; 18D, neutral pH) or the phagolysosome (FIG. 18A; 18C; acidic pH). Bacterial surface zeta potential increased positively with increasing polymer dose regardless of buffer or mannosylation. Hybrid vectors possessed the highest zeta potential values in the acidic condition, which presumably occurs due to protonation of amines on the polymer backbone. Generally, the mannosylated polymers possessed innate zeta potentials lower than their non-mannosylated variants (Table 4), yet mannosylated polymers used to generate hybrid vectors mediated statistically higher zeta potential values regardless of buffer. This may be the result of increased surface deposition (FIGS. 17A-17D) that is driven by yet unknown mechanisms.

Initial Polymer Gene Delivery and Cytotoxicity

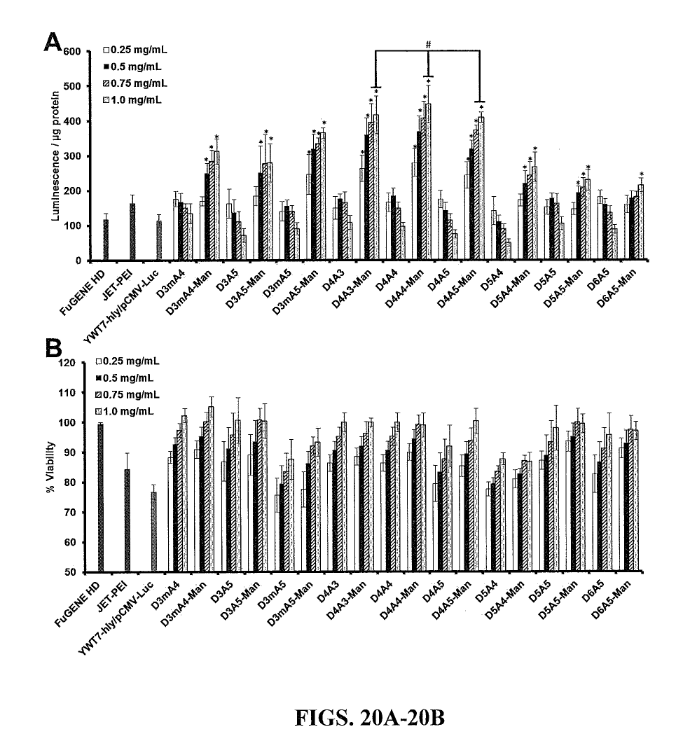

Given the order of magnitude size differences between polyplexes (<300 nm) and hybrid vectors (˜2.5 μm), the effect of upon hybrid-mediated gene delivery was believed to provide a secondary mechanism to promote APCs uptake specificity. In this study, hybrid vectors were evaluated for gene delivery outcomes at a 10:1 MOI and four polymer doses (FIG. 20A).

Mannosylated hybrid gene delivery increased linearly with polymer dose; whereas, non-mannosylated vectors retained optimal activity at lower doses (<0.5 mg/mL). All mannosylated hybrids mediated statistical higher gene delivery values than the commercial and bacterial controls at the highest polymer dose. From this library, hybrid vectors containing D4A4-Man promoted the highest gene delivery values at every polymer dose. The differences between gene delivery outcomes between each polymer were unexpected because of the chemical background similarity across the library. Specifically, monomers were chosen from an extensive chemical background screen and only differ by single carbon displacements. Aside from gene delivery, polymer addition improved hybrid-mediated cytotoxicity in a dose-dependent manner (FIG. 20B) and with exception of a few polymers (D3mA5, D5A4, and D5A4-Man), mediated negligible cytotoxicity (>95% viability) at the highest polymer dose. Decreased cytotoxicity is the result of positive polymer degradation properties (Jones, et al., Biomaterials, 37:333-44 (2015); Jones, et al., Biomacromolecules. (2015); DOI: 10.1021/acs.biomac.5b00062), and charge-mediated bacterial attenuation (Jones, et al., Mol Pharm; 12:846-56 (2015)).

Given the nuanced structural differences between individual polymers, the library was analyzed for a molecular weight effect upon hybrid vector gene delivery. Polymer molecular weight was weakly correlated (R2<0.5) with gene delivery. Furthermore, due to the small size of the library assessed coupled to an incomplete understanding of the fundamental APC-associated vector processing steps, limited information can be ascertained regarding the polymeric structural features governing hybrid vector gene delivery results. Thus, selecting D4A4-Man as the base chemical background (the most effective polymer above), a library of second-generation stratified molecular weight polymers (Table 2 and Figure S3) was synthesized. By doing so, the library can be assessed for structure-function relationships by systematically characterizing gene delivery as a function of: (1) polymer molecular weight; (2) relative mannose content; (3) polymer-membrane biophysical properties; (4) APC uptake specificity; and (5) serum inhibition.

Expanded D4A4-Man Library Assessments

The expanded D4A4-Man library introduces the same chemical background to each polymer, enabling the direct assessment of structural parameters that include polymer molecular weight and mannosylation in hybrid vector formation and subsequent gene delivery outcomes. Using the same transfection strategy described above, hybrid vectors were formed using four polymer doses and a 10:1 MOI (FIG. 20). Hybrid gene delivery increased linearly in reference to increasing polymer dose and decreasing molecular weight. Interestingly, polymers with numbered molecular weights below 10 kDa (P7-P14) mediated gene delivery values statistically higher than commercial and bacterial controls at all polymer doses. In addition, gene delivery gradually increased with molecular weight decreases before plateauing at 5.5 kDa. Below this polymer molecular weight, there are no statistical differences between polymers, regardless of dose, in gene delivery.

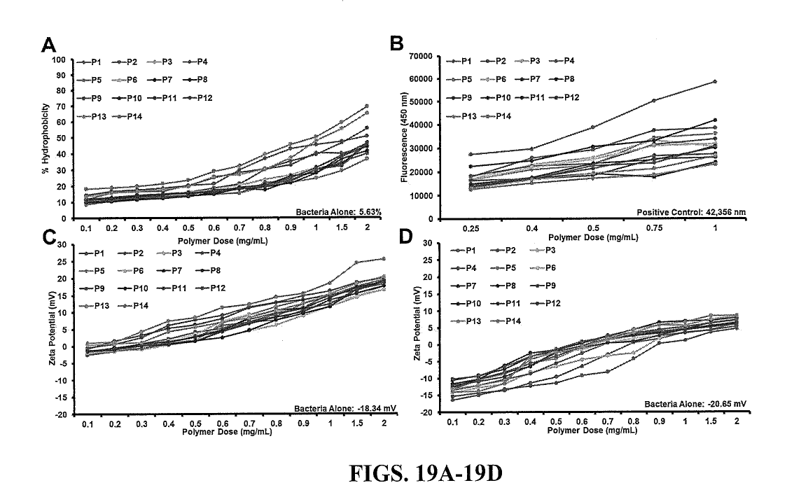

To aid the understanding of the observed gene delivery trends, biophysical characterization was conducted as described above to evaluate the effects of polymer molecular weight on bacterial surface properties (FIGS. 19A-19B). Bacterial membrane coverage (FIG. 19A) and integrity (FIG. 19B) were governed by inverse polymer molecular weight trends. Specifically, lower weight polymers mediated the highest surface coverage; whereas, higher weight polymers mediated the highest membrane destabilization. These observations may be the result of concomitant increases in membrane destabilization potential with increasing polymer molecular weight. This hypothesis is based by the understanding that higher molecular weight polymers possess the highest amine content, which, when introduced to an acidic environment (e.g., the hybrid vector complexation buffer [25 mM NaOAc, pH 5.15]), prompts charge-dependent lipid bilayer disruption similar to other bactericidal agents (e.g., polymyxin B) (Jones, et al. Mol Pharm. 10:4301-4308 (2013); Zhu, et al. Adv Mater, 25:1203-8 (2013)).

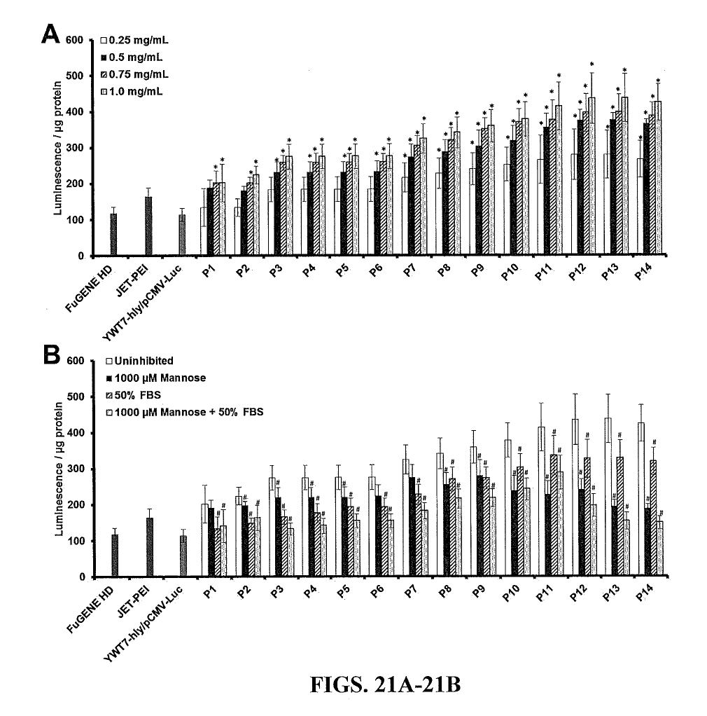

In order for the mannosylated hybrid to be successful in translational applications, these vectors must possess the innate ability to overcome the barriers present during administration. That stated, key limitations include target specificity and the documented loss-of-activity that accompanies vectors in the presence of physiological levels of extracellular protein (Jones, et al., Mol Pharm. 10:4082-4098(2013)). Failure of vectors to traverse these hurdles results in significant off-target effects, premature aggregation, degradation, and subsequent clearance Canine, et al., Adv Drug Deliv Rev. 62:1524-9 (2010); Pack, et al., Nat Rev Drug Discov. 4:581-93(2005)). As such, transfection was conducted using 1.0 mg/mL hybrids at 10:1 MOI in the presence of CD206-inhibiting concentrations of free mannose and/or physiologically-relevant level of serum (FIG. 21B). All hybrids exhibited significant drops in transfection due to both mannose and serum inhibitions. However, decreasing polymer molecular weight resulted in the formation of hybrids that were increasingly sensitive to mannose inhibition and decreasingly sensitive to serum inhibition. Furthermore, in the simultaneous presence of mannose and serum, inhibition resulted in a maximization effect of gene delivery. Specifically, moderate molecular weights (P8-P11) retained gene delivery capabilities surpassing optimal commercial and bacterial controls (transfected in non-physiological conditions; thus these control values represent maximums) This may be the result of a balancing effect between excess charge density present on higher molecular weight polymers (which prompts increased serum deposition due to electrostatic interactions) and increased relative mannose content of lower molecular weight polymers (per unit volume). Collectively, these results suggest translational APC-targeting hybrid vectors should be designed to include moderately charged and moderate molecular weight variants such as P11.

It has been established that mannose-mediated uptake results in endocytic processing that was more permissive to gene delivery. Furthermore, mannose-mediated processing (through CD206 mechanisms) is associated with endocytic trafficking towards recycling mechanisms and through proposed additional endocytic vesicles (early to late endosome). It has also been established that increased efficacy may have also been the result of triggering uptake and processing mechanisms that were either bypassing degradative compartments of the cell (Shin, et al., Science. 293:1447-8 (2001); Pelkmans, et al., Traffic. 3:311-20 (2002); Parton, et al. Nature Reviews Molecular Cell Biology. 8:185-94(2007); Rehman, et al., J Control Release. 166:46-56 (2013)) or escaping endocytic recycling mechanisms Sahay, et al. Nature Biotechnology. (2013)).

However, given the size associated with the hybrid device, uptake is restricted to phagocytic mechanisms and cannot take advantage of general uptake. Interestingly, other studies have highlighted the harsh nature of processing through general phagocytosis, indicating minor biomolecule release representing less than 5% of processed cargo (Gage, et al., Toxins. 3:1131-45(2011)).

Thus, the role of mannosylation upon hybrid-mediated processing is not completely clear. That said, mannosylation of hybrids is triggering uptake and processing mechanisms beneficial to eventual gene delivery that may not be governed by the same polymeric structure-function rules associated with general uptake mechanisms. Nevertheless, increased gene delivery activity may be rooted in the nature of the mannose receptor (MR). Even though MR-mediated phagocytosis is poorly understood, the innate activity of this processing pathway is directed against pathogenic microbes which are coated with mannose-containing structures (Medzhitov, Nature 449:819-26 (2007)). Furthermore, instigation of this receptor is known to increase antigen presentation activity in vaccine efforts Carrillo-Conde, et al. Mol Pharm. 8:1877-86 (2011); Chavez-Santoscoy, et al., Biomaterials. 33:4762-72 (2012); Prigozy, et al. Immunity. 6:187-97 (1997)) by intensifying recruitment of degradative components (e.g., lysozyme and proteases). However, intracellular pathogens are known to take advantage of the increased degradative activity by the over-expression of pH-dependent hemolysin proteins Stahl, et al. Current Opinion in Immunology 10:50-5 (1998); Parsa; et al., Mol Pharm.; 4:4-17 (2007)). Given the current design of the bacterial-component of the hybrid vector to heterologously express the pH-dependent hemolysin, LLO, increased degradative activity should increase resulting gene delivery responses. The combination of mannosylation with the engineered bacterial core provides an additional demonstration of the dual flexibility in the generation of hybrid vectors that possess new levels of biomimicry.

In summary, the library of polymers developed in this study highlights the ability of moderately sized and charged mannosylated PBAEs to form hybrid bio-synthetic vectors that are capable of mediating effective transfection to APCs in physiological conditions. Taken together, the study provides the first report of polymer structure-function relationships that can be readily applied to all future hybrid bio-synthetic gene delivery studies.

### Example 8: Pegylation of Cationic Polymers Enhances Delivery of Nucleic Acids by Hybrid Bacterial Vectors

### Methods

To elucidate the underlying properties responsible for the observed efficacy of hybrid bio-synthetic gene delivery vectors, a set of well-defined PEGylated and unPEGylated cationic polylactides with tunable charge densities were utilized as the polymer constituent. Effects of charge density and PEGylation on hybrid characteristics and gene delivery efficacy were systematically studied as was the effect of polymer addition upon hemolysis and immunogenicity.

### Measurements

¹H-NMR spectra were measured at 500 MHz in CDCl₃ using a

Varian INOVA-500 spectrometer maintained at 25° C. with tetramethylsilane (TMS) as an internal reference standard.

Gel permeation chromatography (GPC) data were acquired from a Viscotek GPC system equipped with a VE-3580 refractive index (RI) detector, a VE 1122 pump, and two mixed-bed organic columns (PAS-103M and PAS-105M). Dimethylformamide (DMF; HPLC) containing 0.01 M LiBr was used as mobile phase with a flow rate of 0.5 mL/min at 55° C. The GPC instrument was calibrated using narrowly-dispersed linear polystyrene standards purchased from Varian.

Zeta potential of hybrid vectors were obtained using dynamic light scattering (DLS) on a Zetasizer nano-ZS90 (Malvern, Inc.) in water at 25° C. All experiments were conducted using a 4 mW 633 nm HeNe laser as the light source at a fixed measuring angle of 90° to the incident laser beam. The correlation decay functions were analyzed by cumulants method coupled with Mie theory to obtain volume distribution.

### Materials

4-Dimethylaminopyridine (DMAP; 99+%) and L-lactide (L-LA, 98%) were purchased from Sigma-Aldrich. 2,2′-Dimethoxy-2-phenylacetophenone (DMPA; 98%) was purchased from Acros Organics. Dichloromethane (DCM; HPLC), acetone (HPLC), ethyl acetate (HPLC), n-hexadecane (HPLC), DMF (HPLC), and diethyl ether (HPLC) were purchased from Fisher Chemical. α-Methoxy-ω-hydroxyl polyethylene glycol (mPEG-OH; MW: 2,000 Da) was purchased from RAPP Polymere. 2-(Diethylamino)ethanethiol hydrochloride (DEAET, >98%) was purchased from Amfinecom Inc. DCM, DMF and ethyl acetate were dried by distillation over CaH₂. LA was recrystallized from dry ethyl acetate four times prior to use. mPEG-OH was dried as follows prior to use: mPEG-OH was dissolved in 1 mL dried DCM, followed by complete solvent removal, and the cycle was repeated five times; toluene was used as a solvent to treat mPEG-OH for another five cycles. Allyl-functionalized LA monomer (#1 in scheme IV, below) was prepared through the method reported by Chen, et al., Adv Healthc Mater, 1, (6), 751-61 (2012). All other chemicals were used without further purification.

Synthesis of Allyl-functionalized PLA (2a) and CPLA-26 and CPLA-54

Allyl-functional PLA (2) was synthesized according to the scheme IV. Briefly, 1 (1,440 mg; 10 mmol), L-LA (1,700 mg; 10 mmol), and DCM (16.3 mL) were added to a 25 mL reaction flask with a magnetic stirring bar under nitrogen atmosphere. Upon reaching a solution temperature of 35° C., BnOH (21.6 mg; 0.2 mmol; in 0.5 mL DCM) and DMAP (97.7 mg; 0.8 mmol; in 0.5 mL DCM) were added to initiate the polymerization. Synthesis was allowed to continue for 3 weeks at 35° C., before being manually stopped at a co-monomer conversion of ˜80%. Co-monomer conversion was calculated by ¹H-NMR based on the resonance intensities of the CH₃ protons of remaining co-monomers at 1.67-1.73 ppm relative to the CH₃ protons of the resulting polymer at 1.49-1.61 ppm. Next, allyl-functionalized 2a was purified by precipitation in ice-cold methanol (50 mL) (see scheme IV).

TABLE 6
Synthesis of CPLA and PEGylated variants

| | | Amine | Mₙ^{NMR} | |
|---|---|---|---|---|
| Polymer | [ene]₀:[SH]₀:[DMPA]₀ | (mol %)^{[a]} | (kDa) | PDI^{GPC[b]} |
| CPLA-26 | 1:0.5:0.2 | 26 | 18.5 | 1.37 |
| PEG-b- | 1:0.5:0.2 | 20 | 6.2 | 1.06 |
| CPLA-20 | | | | |
| CPLA-54 | 1:3:0.4 | 54 | 22.8 | 1.35 |
| PEG-b- | 1:3:0.4 | 50 | 7.3 | 1.06 |
| CPLA-50 | | | | |

^{[a]}Determined by ¹H NMR spectroscopy relative to repeat units of the CPLA block.
^{[b]}Relative to linear polystyrene standards.

¹H NMR (500 MHz, CDCl₃, ppm) of 2a: δ 1.49-1.61 (br m, CH₃ units from LA and 1), 2.66-2.73 (br m, CH₂CH═CH₂ units from 1), 5.14-5.23 (br m, CHCH₃ units from LA; CHCH₃, CHCH₂CH═CH₂, and CH₂CH═CH₂ units from 1), 5.77-5.79 (m, CH₂CH═CH₂ units from 1), 7.33-7.39 (m, Ar—H from BnOH). Mₙ^{NMR}=14.5 kDa; Mₙ^{GPc}=21.9 kDa; PDI^{GPC}=1.12. Mole fraction of 1 was 54% based upon the ¹H NMR resonance intensities of 1H from units of 1 at 5.77-5.79 ppm relative to 4H from units of 1 and 2H from units of LA at 5.14-5.23 ppm.

To synthesize CPLA-26, 2a (200 mg), DEAET (57.3 mg), and photo-initiator DMPA (34.8 mg) were dissolved in CDCl₃ (5 mL) in a 10 mL flask, resulting in the molar ratio of [allyl of 2a]₀:[SH of DEAET]₀:[DMPA]₀=1:0.5:0.2. The freeze-pump-thaw procedure was conducted for three cycles to deoxygenate the solution. Then, the thiol-ene reaction was induced by UV irradiation (λₘₐₓ=365 nm) for 30 min. Subsequently, to remove the unreacted DEAET and DMPA, dialysis of the resulting solution was conducted against acetone for 10 days using molecular porous membrane tubing (Spectra/Por Dialysis Membrane, Spectrum Laboratories Inc.) with an approximate molecular weight cut off (MWCO) at 3,500 Da. Following dialysis, the solution was completely dried by vacuum to give CPLA-26 at 90% yield. Using a different feed ratio of reactants ([allyl of 2a]₀: [SH of DETA]₀:[DMPA]₀=1:3:0.4), CPLA-54 was prepared using the same method applied to CPLA-26

¹H NMR (500 MHz, CDCl₃, ppm) of CPLA-26: δ 1.39-1.43 (br m, (CH₃CH₂)₂NH⁺Cl⁻ from amine-functionalized units), 1.49-1.61 (br m, CH₃ from LA, 1, and amine-functionalized units), 1.76-1.79 (br m, CH₂CH₂CH₂SCH₂ from amine-functionalized units), 2.00-2.10 (br m, CH₂CH₂CH₂SCH₂ from amine-functionalized units), 2.49-3.23 (br m, CH₂CH═CH₂ units from 1; CH₂CH₂CH₂SCH₂ and SCH₂CH₂NH⁺Cl⁻ (CH₂CH₃)₂ from amine-functionalized units), 5.14-5.20 (br m, CHCH₃ units from LA; CHCH₃, CHCH₂CH═CH₂, and CH₂CH═CH₂ units from 1; CHCH₃ units from 2a; and CHCH₂CH₂CH₂SCH₂ from amine-functionalized units), 5.77-5.79 (br m, CH₂CH═CH₂ units from 1), 7.33-7.39 (m, Ar—H from BnOH). Mₙ^{NMR}=18.5 kDa; Mₙ^{GPc}=22.9 kDa; PDI^{GPc}=1.37. ¹H NMR (500 MHz, CDCl₃, ppm) of CPLA-54: δ 1.17-1.26 (br m, (CH₃CH₂)₂NH⁺Cl⁻ from amine-functionalized units), 1.49-1.61 (br m, CH₃ from LA and amine-functionalized units), 1.74-1.77 (br m, CH₂CH₂CH₂SCH₂ from amine-functionalized units), 2.00-2.08 (br m, CH₂CH₂CH₂SCH₂ from amine-functionalized units), 2.49-3.20 (br m CH₂CH₂CH₂SCH₂ and SCH₂CH₂NH⁺Cl⁻ (CH₂CH₃)₂ from amine-functionalized units), 5.14-5.20 (br m, CHCH₃ units from LA; CHCH₃ and CHCH₂CH₂CH₂SCH₂ from amine-functionalized units), 7.33-7.39 (m, Ar—H from BnOH). Mₙ^{NMR}=22.8 kDa; Mₙ^{GPc}=16.4 kDa; PDI^{GPC}=1.35.

Synthesis of PEG-b-allyl-functionalized PLA (2b) and PEG-b-CPLAs

In a 10 mL reaction flask with a magnetic stirring bar under nitrogen atmosphere, 1 (544 mg; 3.78 mmol), L-LA (643 mg; 3.78 mmol), and dried mPEG-OH (189 mg; 0.09 mmol) were added with dried DCM (4 mL). The solution was heated to 35° C. using an oil bath for 1 h, followed by the addition of a solution of DMAP (44 mg; 0.36 mmol) in 0.5 mL of dried DCM. After incubation for 1 week at 35° C., the reaction was stopped at co-monomer conversion of ˜80% as determined by ¹H NMR analysis of an aliquot of polymerization solution, based on the resonance intensities of the CH₃ protons of remaining co-monomers at 1.67-1.71 ppm relative to the CH₃ protons of the resulting polymer at 1.49-1.59 ppm. The reaction mixture was precipitated by cold diethyl ether three times. Then the precipitate was collected and dried in a vacuum to give 2b as a white solid powder in 30% isolated yield. ¹H NMR (500 MHz, CDCl₃, ppm): δ 1.49-1.59 (br m, CH₃ units from LA and 1), 2.67-2.73 (br m, CH₂CH═CH₂ units from 1), 3.38 (s, terminal CH₃O units of mPEG-OH), 3.54-3.68 (br m, CH₂O units of mPEG-OH), 5.14-5.30 (br m, CHCH₃ units from LA; CHCH₃, CHCH₂CH═CH₂ and CH₂CH═CH₂ units from 1), 5.77-5.79 (m, CH₂CH═CH₂ units from 1). Mₙ^{NMR}=5.5 kDa, Mₙ^{GPC}=14.0 kDa, PDI^{GPC}=1.05. The mole fraction of 1 in the PLA-based block was 50% based upon the ¹H NMR resonance intensities of 1H from units of 1 at 5.77-5.79 ppm relative to 4H from units of 1 and 2H from units of LA at 5.14-5.30 ppm.

For the synthesis of PEG-b-CPLA-20, in a 10 mL flask, 2b (100 mg), DEAET (16.7 mg), and photoinitiator DMPA (10.15 mg) were dissolved in CDCl₃ (5 mL) with molar ratio of [allyl of 2b]₀:[SH of DEAET]₀:[DMPA]₀=1:0.5:0.2. To remove oxygen in the reaction solution, a freeze-pump-thaw procedure was carried out for three cycles. Subsequently, the thiol-ene reaction was induced by UV irradiation (λₘₐₓ=365 nm) for 30 min.

The reaction solution was dialyzed against acetone for five days using molecular porous membrane tubing (as described above for non-PEGylated CPLAs). Drying of the resulting solution in vacuum gave PEG-b-CPLA-20 with 87% yield. ¹H NMR (500 MHz, CDCl₃, ppm) of PEG-b-CPLA-20: δ 1.28-1.36 (br m, (CH₃CH₂)₂NH⁺Cl⁻ from amine-functionalized units), 1.51-1.63 (br m, CH₃ from LA, 1, and amine-functionalized units), 1.76-1.81 (br m, CH₂CH₂CH₂SCH₂ from amine-functionalized units), 2.00-2.04 (br m, CH₂CH₂CH₂SCH₂ from amine-functionalized units), 2.64-3.20 (br m, CH₂CH═CH₂ units from 1; CH₂CH₂CH₂SCH₂ and SCH₂CH₂NH⁺Cl⁻ (CH₂CH₃)₂ from amine-functionalized units), 3.40 (s, terminal CH₃O of mPEG-OH), 3.53-3.68 (br m, CH₂O of mPEG-OH), 5.13-5.30 (br m, CHCH₃ units from LA; CHCH₃, CHCH₂CH═CH₂, and CH₂CH═CH₂ units from 1; CHCH₃ units from 2b; CHCH₂CH₂CH₂S from amine-functionalized units), 5.79-5.83 (br m, CH₂CH═CH₂ of units from 1). Mₙ^{NMR}=6.2 kDa, Mₙ^{GPC}=14.3 kDa, PDI^{GPc}=1.06.

Using a different feed ratio of reactants ([allyl of 2b]₀:[SH of DETA]₀:[DMPA]₀=1:3:0.4), PEG-b-CPLA-50 was prepared using the same method applied to PEG-b-CPLA-20. ¹H NMR (500 MHz, CDCl₃, ppm) of PEG-b-CPLA-50: δ 1.28-1.37 (br m, (CH₃CH₂)₂NH⁺Cl⁻ from amine-functionalized units), 1.53-1.63 (br m, CH₃ from LA and amine-functionalized units), 1.76-1.82 (br m, CH₂CH₂CH₂SCH₂ from amine-functionalized units), 2.00-2.18 (br m, CH₂CH₂CH₂SCH₂ from amine-functionalized units), 2.60-2.72 (br m, CH₂CH₂CH₂SCH₂ from amine-functionalized units), 2.80-3.20 (SCH₂CH₂NH⁺Cl⁻ (CH₂CH₃)₂ from amine-functionalized units), 3.40 (s, terminal CH₃O of mPEG-OH), 3.53-3.68 (br m, CH₂O of mPEG-OH), 5.12-5.25 (br m, CHCH₃ units from LA; CHCH₃ units from 2b; CHCH₂CH₂CH₂S from amine-functionalized units). Mₙ^{NMR}=7.3 kDa, Mₙ^{GPC}=14.0 kDa, PDI^{GPc}=1.06.

Preparation of Gene Delivery Vectors

Bacterial and hybrid vectors were prepared from bacterial cultures inoculated at 2% (v/v) from overnight starter cultures. Plasmid selection antibiotics were used as needed during bacterial culture within lysogeny broth (LB) medium. Following incubation at 36° C. and 250 rpm until 0.4 to 0.5 OD₆₀₀, samples were induced with 0.1 mM isopropyl β-D-1-thiogalactopyranoside (IPTG) at 30° C. for 1 hr. Bacterial vectors were then washed once and standardized to 0.5 OD₆₀₀ in PBS; whereas, bacterial strains to be used in hybrid vector formation were washed once and standardized to 1.0 OD₆₀₀ in 25 mM NaOAc (pH 5.15). Polymer doses dissolved in chloroform were desiccated and resuspended in 25 mM NaOAc (pH 5.15) prior to equal volume addition to 1.0 OD₆₀₀ bacterial strains. Hybrid vectors (final 0.5 OD₆₀₀) and bacterial vectors in PBS were allowed to incubate at 22° C. for 15 minutes before being diluted into RPMI medium to produce desired multiplicity of infections (MOIs; ratio of the number of hybrid vectors to APCs).

Transfection Studies

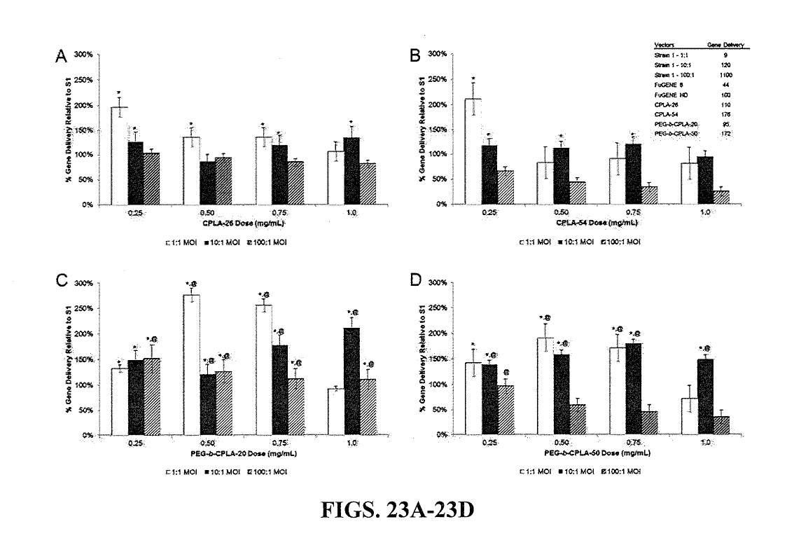

Transfection studies were carried out as described above. Hybrid vectors composed of all four polymers at four doses were incubated with a murine macrophage cell line, RAW264.7, and assessed for luminescence (FIG. 23). Gene delivery is reported as quotient of luminescence to total protein content of each respective sample. In addition, these values are further standardized by gene delivery values of S1. As such, values exceeding 100% represent improvements upon gene delivery as compared to the bacterial control in isolation.

Serum Inhibition to Transfection

To determine if transfection was affected by the presence of increasing levels of serum, hybrid vectors were prepared and incubated with RAW264.7 cells (100 ng/well) in RPMI-1640 medium with 10, 20, 30, 40, 50, and 60% FBS for 24 h. Gene delivery was quantified as described above.

MTT Assay and Nitric Oxide Production

Cytotoxicity resulting from hybrid vectors was deteiinined by the MTT colorimetric assay as described above.

Statistical Evaluation

Unless otherwise indicated, data presented were generated from three independent experiments. Error bars represent standard deviation values. All statistical significance comparisons were performed using a one-way ANOVA with Dunnett (to compare within groups) or Bonferroni (to compare across groups) post-tests.

### Results

It has been established PBAEs can be used for surface modification of bacterial cells. This class of polymers is recognized for ease of synthesis and significant transfection levels. However, such polymer classes possess relatively large PDI values (>1.4) that result in potential batch-to-batch variation of transfection and cytotoxicity responses. It has also been established that targeting moieties (i.e., mannose) to polymer backbones reduces coalescence at elevated polymer doses.

These data investigate the effect of the most commonly used shielding agent, poly(ethylene glycol) (PEG). These experiments thus investigate the utilization of a class of well-defined PEGylated and unPEGylated cationic polylactides (PEG-b-CPLA and CPLA) with varying charge densities.

Polymers were synthesized through living ring-opening polymerization and click functionalization (Scheme 1 and Table 6). It has been established that Amine mol % of CPLAs and PEG-b-CPLAs was the most important structural factor to govern their hydrolysis rate, complexation ability with genetic material, and the transfection efficiency of the corresponding polymer-genetic material nanoplexes.

Therefore, CPLAs and their PEG-b-CPLA counterparts with similar amine mol % were selected as the cationic polymer components in the current study.

The formation of hybrid bio-synthetic gene delivery vectors is presumably driven by electrostatic interactions between positively charged polymers and the negatively charged outer membrane of E. coli, which permits the use of simple mixing schemes. This facile method of formulation is advantageous for future scalability studies as it eliminates complex formulation protocols and can be accomplished without the use of expensive equipment. Furthermore, for all presented studies involving hybrid vectors, formulations were prepared over a range of polymer doses (0.25, 0.5, 0.75, and 1.00 mg/mL) to assess the impact degree of coating has upon subsequent results.

A singular E. coli strain was selected as the optimal choice to deliver a mammalian expression luciferase reporter plasmid based upon optimization studies conducted previously (Jones, et al., Mol Pharm, 10, (11), 4301-4308 (2013); Parsa, et al., J. Biotechnol., 137, (1-4), 59-64 (2008); Parsa, et al., Pharm. Res., 25, 1202-1208 (2008)). The selected strain, YWT7-hly/pCMV-Luc, (S1) contains an inducible LLO expression cassette (T7 promoter driven) chromosomally integrated into BL21(DE3) at the clpP gene location.

Polymer-mediated bacterial extracellular release of protein and DNA was observed in a dose-dependent manner, suggesting revised structural features of the final hybrid device. Transfection with PEGylated hybrids demonstrated statistical improvements when compared to unaltered bacterial controls. In addition, gene delivery maintained in elevated levels of serum highlighted the positive effects of CPLA PEGylation when compared to unPEGylated hybrid vectors and points to the potential of utilizing shielding agents for future hybrid vector-mediated delivery applications.

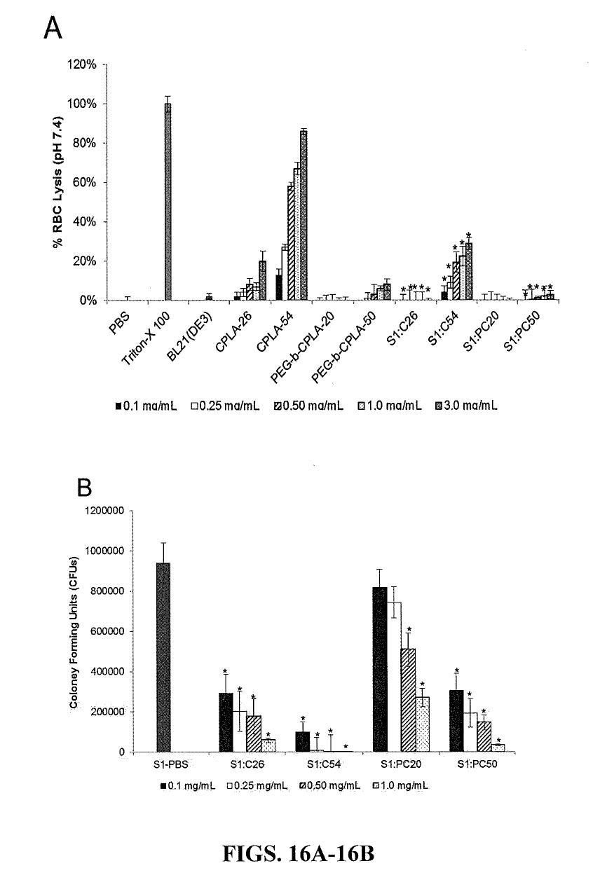

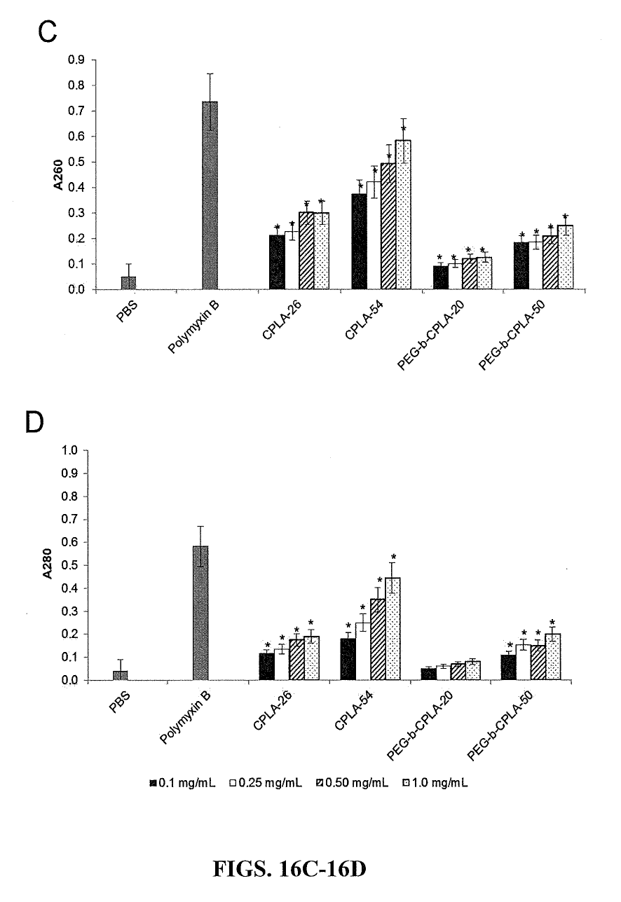

Hemolysis was investigated in the context of the hybrid device and vectors in isolation (FIG. 16A). Incubation of polymers with RBCs resulted in dose-dependent hemolysis; whereas, PEGylated polymers possessed the same trend but at statistically significant lower values. Conversely, incubation with the background strain of bacteria (BL21[DE3]) in isolation resulted in no apparent hemolysis (<2%).

Upon hybrid vector formation, statistically significant reductions of hemolysis for hybrids formulated with CPLA-26 and -54 (S1:C26 and S1:C54) and PEG-b-CPLA-20 and -50 (S1:PC20 and S1:P50) were observed for all concentrations. Reduction in hybrid-mediated hemolysis is presumably caused by polymer charge neutralization and suggests that higher dosages of hybrid vectors can be tolerated in vivo.

Aside from charge neutralization, cationic polymer surface deposition to the bacterial outer membrane may weakly disrupt the phospholipid bilayer in a mechanism similar to pore-fonning antibiotics (e.g., polymyxin B). Shear disruption studies were conducted by briefly sonicating (5 s) strain 1 in PBS (S1-PBS) and respective hybrid vectors (FIG. 16B). With exception of S1:PC20 at lower polymer doses, all hybrid vectors were significantly attenuated in a dose-dependent manner when compared to S1-PBS. Attenuation increased linearly with respect to total charge density (C54>C26>PC50>PC20). Higher doses of CPLA-54 or PEG-b-CPLA-50 may potentially result in two opposing effects. Specifically, due to the intrinsic properties of the uncoated bacteria (i.e., uncoated bacteria mediate moderate APC cytotoxicity), polymer-mediated membrane destabilization of the bacterial cell wall has been previously linked to improvements upon APC gene delivery and cell viability; alternatively, the increased fragility of the bacterial membrane in the hybrid device may prompt premature clearance and/or vector decomposition. Furthermore, improvements to APC gene delivery and cytotoxicity resulting from bacterial membrane disruption are linked to the leakage of intracellular material. For instance, upon APC internalization, increased leakage of protein, specifically LLO, and plasmid DNA (pDNA) can further improve gene delivery by enhancing phagosomal escape and the concentration of genetic cargo available for transfection, respectively.

pDNA and protein release studies were completed using hybrid vectors prepared as described above and compared to analogous bacterial treatments with PBS (negative control) and polymyxin B (positive control) (FIGS. 16C-16D). Hybrid vectors demonstrated a statistically significant CPLA-mediated dose-dependent increase in release for both pDNA (A260) and protein (A280) for all polymers with the exception of PEG-b-CPLA-20 for protein release. S1:PC20 demonstrated the least attenuation potential which may be correlated by the small molecular weight and/or the lack of increased charge density. Given the lack of bacterial core attenuation, this vector should only be utilized in a context where a stronger immunological response is required.

To further quantify the degree of surface modification, hybrid vectors were assessed for net surface charge using DLS. Bacterial surface charge transitioned to increasingly positively charged states in a dose- and charge density-dependent manner. Furthermore, increasing charge density, C54>PC50>C26>PC20, mediated an increasing net surface charge trend. Analogously, surface modification of bacterial vectors with cationic polymers that possess large stretches of hydrophobic domains were expected to affect resulting hybrid vector polarity, noting that similar vector modifications have been associated with increases in gene delivery.

Thus, using a MATH assay, hybrid vectors were assessed for increased relative hydrophobicity as compared to untreated bacterial controls. All polymers resulted in a general dose-dependent increase upon surface hydrophobicity. No statistically significant trends emerged related to charge density except at the lowest dose (0.25 mg/mL). At this dose, bacterial surface modification by CPLAs is driven predominately by charge difference between hybrid constituents. Thus, polymers with higher charge density are expected to provide better coverage at these polymer doses, leading to greater hydrophobicity measurements of the hybrid devices as a result. As coating increases with greater polymer dosing, the hydrophobicity levels between hybrid vectors are not statistically different.

Surface addition of cationic polymers results in the permeation of bacteria without causing gross bactericidal effects. The initial interaction between the bacterial surface and polymers is driven by electrostatic interactions before being replaced by membrane integration mechanisms.

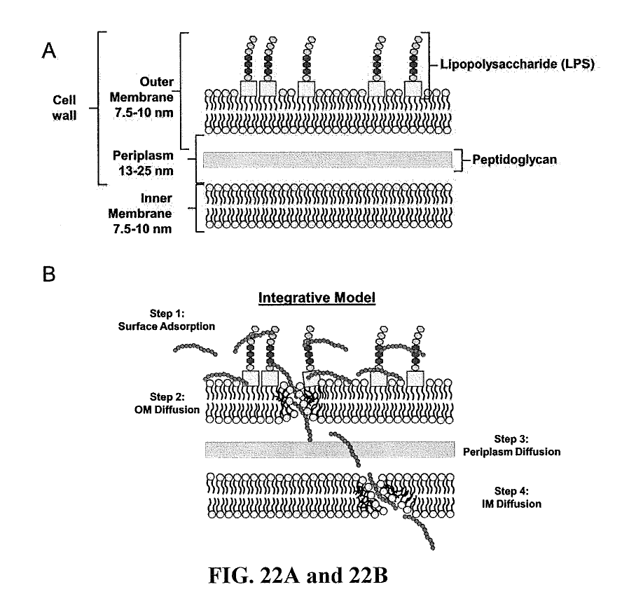

This “integrative” hypothesis stems from observations that polymers have both surface and membrane-spanning effects (FIGS. 22A-22B). Surface coverage is supported by visual hybrid vector coalescence to form a larger biofilm-like structure upon increased doses of polymer and that NO production mediated by binding of LPS to external receptors of macrophages is significantly reduced upon polymer addition. Conversely, membrane-spanning effects include the extracellular release of protein and pDNA upon polymer addition which would require double membrane permeation of the outer and inner phospholipid bilayers.

Furthermore, given the estimated thickness of 30-40 nm for an intact Gram-negative bacterial surface (7.5-10 nm for each inner and outer membrane and 13-25 nm for the periplasmic space), it is unlikely that polymer molecules can span the entire space with the consideration of their molecular sizes. In addition, the mechanism of disruption most-likely resembles cationic antimicrobial polymers that act through a sequential series of steps. Accordingly, the first step presumably involves the initial surface adsorption (mediated primarily through charge-charge interactions), followed by diffusion and mild disruption of the outer membrane. Lastly, upon diffusion through the outer membrane and the peptidoglycan layer, the polymer may again adsorb on the inner membrane before diffusing and disrupting (as before) the inner membrane.

Upon confirming bacterial surface modification and possible polymer-membrane integration, hybrid vectors were evaluated for gene delivery capabilities using a luciferase reporter model. Hybrid vectors composed of all four polymers at four doses were incubated with a murine macrophage cell line, RAW264.7, and assessed for luminescence (FIG. 23). Gene delivery is reported as quotient of luminescence to total protein content of each respective sample. In addition, these values are further standardized by gene delivery values of S1. As such, values exceeding 100% represent improvements upon gene delivery as compared to the bacterial control in isolation.

Hybrids composed of unPEGylated CPLAs (FIGS. 23A and 23B) demonstrated a generally negative gene delivery correlation with respect to dose increases. In contrast, PEGylated hybrids showed less of an overall trend but demonstrated improved gene delivery with increased polymer dose for 1:1 and 10:1 MOI samples (FIGS. 23C and 23D). As a group, PEGylated hybrids resulted in gene delivery values that were improved in comparison to their unPEGylated counterparts. Interestingly, the S1:PC20 hybrid demonstrates the greatest gene delivery values. In comparison to the trends of FIG. 16, the biophysical properties of the S1:PC20 hybrid enabling improved gene delivery at the indicated polymer dose levels. In addition, reductions of hybrid-mediated gene delivery at doses higher than 0.5 mg/mL were observed. This was caused by coalescence of hybrid vectors into conglomerates that were unable to be phagocytized and/or processed. The results presented here suggest that the addition of a shielding functionality prevent undesired coalescence, thus increasing the number of viable vectors per unit volume with increased polymer doses.

Serum Inhibition of Transfection

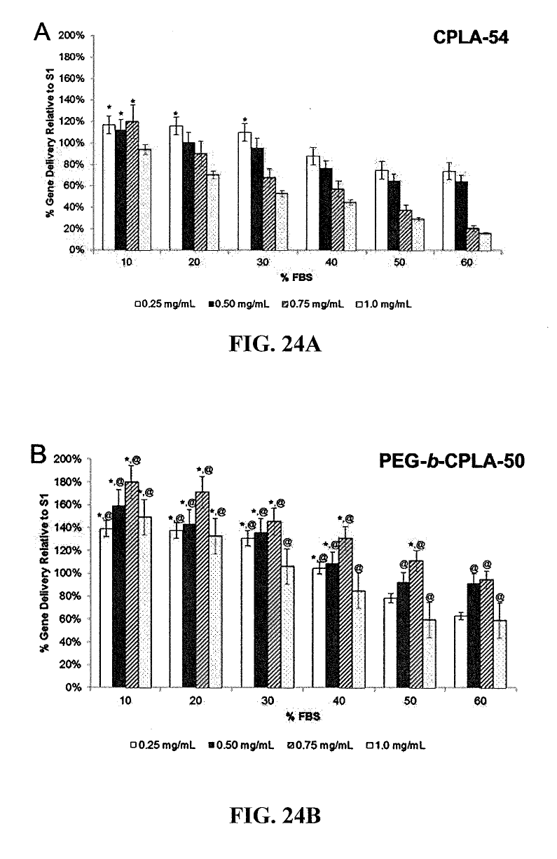

For PEG-b-CPLA-based hybrid vectors to be relevant in in vivo applications, gene delivery must maintain efficacy in the presence of high concentrations of protein. The marked decrease of efficacy of transfection agents when shifting from in vitro models to an in vivo application is due to the deposition of negatively charged serum proteins to positively charged complexes, which in turn, results in aggregation and clearance. As such, CPLA-54 and PEG-b-CPLA-50 hybrids were prepared and transfected with RAW264.7 cells with increasing v/v percentages of FBS (FIG. 24). Values were standardized by gene delivery values from transfection using S1 in medium containing 10% FBS (noting transfection conditions). For both polymers, gene delivery was negatively correlated with increased levels of FBS. In addition, CPLA-54 hybrids demonstrated a dose-dependent decrease in gene delivery at all FBS levels except 10% FBS, but gene delivery remained statistically improved until 30% FBS. Higher doses of polymer decreased at a faster rate as compared to the lower doses due to increased deposition of FBS and aggregation. Conversely, PEG-b-CPLA-50 hybrids demonstrated statistically significant improvements in gene delivery until 50% FBS. Interestingly, at 60% FBS, polymer doses 0.50 and 0.75 mg/mL performed comparable to the S1 control in 10% FBS. This is significant because it is generally accepted that physiological serum levels range from 45 to 60% of volume. Unlike the unPEGylated hybrids, PEGylation resulted in a dose-dependent increase of gene delivery, with exception of 1.0 mg/mL, across all FBS levels. Presumably this is associated with the innate properties of PEG to prevent coalescence and aggregation of particles resulting from serum deposition. Taken together, this is the first report indicating the importance of PEGylation (or any shielding molecules) and well-defined structural characteristics of the hybrid vector polymer constituent in preventing drastic reductions of gene delivery that is normally accompanied with increased levels of serum.

Cytotoxicity and NO Production

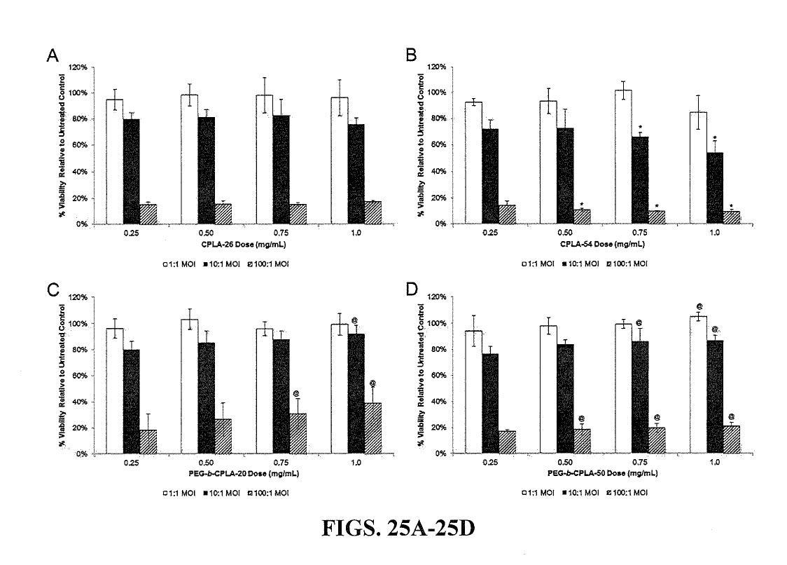

For eventual translation, the hybrid technology must possess a safe cytotoxicity and immunogenicity profile. Thus, CPLA hybrids were examined for their cytotoxicity at four polymer doses and three MOIs (1:1, 10:1, 100:1) (FIG. 25).

At the lowest MOI, CPLA-26 hybrids demonstrated no cytotoxicity; whereas, increased MOI levels were associated with increased cytotoxicity (FIG. 25A). Conversely, use of a higher charge density polymer, CPLA-54 (>2 times the amine content of CPLA-26), resulted in a statistically significant dose-dependent increase in cytotoxicity at higher MOIs and doses when compared to the CPLA-26 hybrid (FIG. 25B). This is likely the result of innate cytotoxicity associated with high-charge density polymers. Jones, et al. Mol Pharm, 10, (3), 1138-45 (2013); Chen, et al., Biomaterials 34, (37), 9688-99 (2013)). Interestingly, formation of hybrids with PEGylated CPLAs resulted in a dose-dependent decrease in cytotoxicity for both polymers across all MOIs. The largest dose-dependent decrease of cytotoxicity occurred with the use of PEG-b-CPLA-20; however, use of PEG-b-CPLA-50 resulted in a significant decrease of cytotoxicity when compared to CPLA-54.

Aside from cytotoxicity, a hybrid vector must reduce unwanted immunogenicity associated with the use of Gram-negative bacteria. As such, a Griess reagent assay was used to assess macrophage activation via lipopolysaccharide (LPS)-mediated NO production.

Incubation of LPS with RAW264.7 cells is linked to concomitant release of TNF-α and NO via binding and activation of toll-like receptor 4 (TLR4). Although APC activation is required to elicit an effective immune response, excessive bacterial-mediated activation can result in systemic shock and potential death NO production resulting from the incubation with hybrids vectors composed of (CPLA-54) and PEG-b-CPLA-50 was investigated. In both cases, polymer coating resulted in significant reduction of NO as compared to bacterial controls across all polymer doses and vector MOIs. Reduction in NO production presumably occurs by physical masking of LPS 20 and/or competitive TLR4 binding. Additional confirmation of polymer coating improvements of cytotoxicity and immunogenicity highlights another advantage of the hybrid vector.

Results indicate that this class of polymers effectively complements hybrid vector design and function and alters the previous model proposed for vector assembly. Accordingly, a new “integrative model” has been developed and presented to better align with experimental observations. In addition, PEGylation prevents coalescence of hybrid particles, thus, providing a means to confer serum resistance and hemolysis reduction.

### Example 9: Immunization Using Hybrid Bacterial Vectors Expressing OVA Peptide Antigen Stimulates an Immune Response in Animals

### Methods

To test the hybrid vector approach in an in vivo setting, completed mouse model assessment of antibody response against the model ovalbumin (OVA) antigen was carried out.

Female BALB/c mice aged 8 weeks were obtained from The Jackson Laboratory (Bar Harbor, Me.) and housed and utilized in accordance with institutional guidelines.

For immunizations, hybrid vectors were prepared as described above and diluted in PBS to 1×10⁵ and 1×10⁷ vectors per 200 μL (total volume). Antigens were emulsified with the adjuvants to yield 1 mg/mL (OVA protein) and 100 μg/mL (OVA pDNA) concentrations. Mice were immunized at days 0 and 14 in sets of six per sample. Hybrid vectors were injected (200 μL) subcutaneously (s.c) and intraperitoneally (i.p), while controls (plasmid and protein) were only injected subcutaneously.

Initial immunization of controls included CFA, which was replaced with IFA during booster immunizations. At days 14 and 21, retro-orbital blood samples were collected and clarified by centrifugation to collect serum. Serum anti-OVA antibody titers were quantified using the Cayman Anti-Ovalbumin IgG1 (mouse) EIA Kit (Caymen Chemical, Ann Arbor, Mich.) according to the manufacturer's instructions. For normalization comparisons, the estimation for pDNA delivered by the hybrid vector was derived from the calculation presented in the footnotes of Table 3 adjusted for pCI-neo-cOVA.

### Results

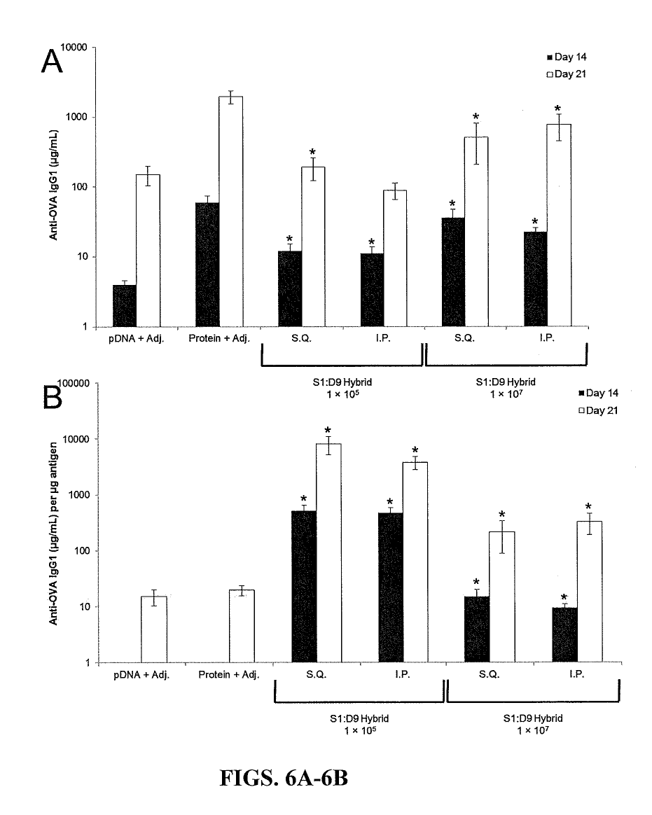

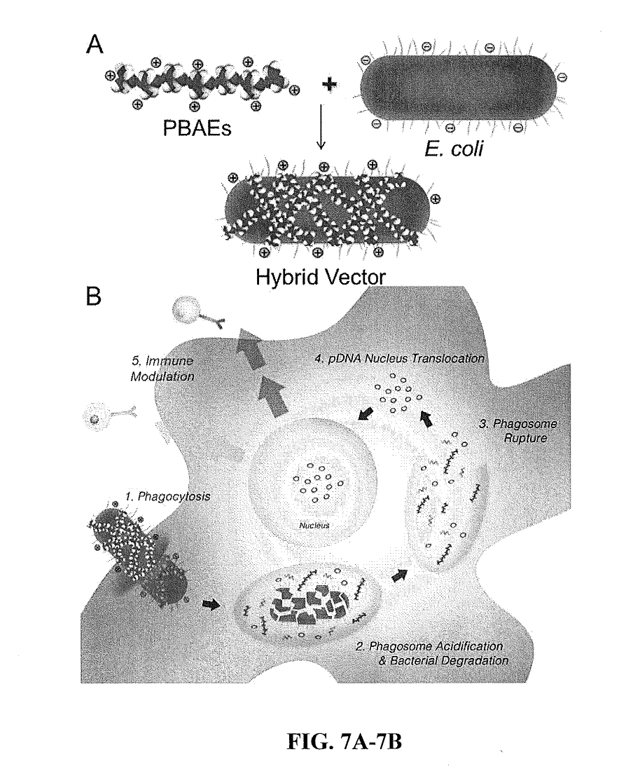

Results are presented in FIGS. 6A-6B. No toxicity was observed amongst the mouse subjects and total IgG1 titers obtained were comparable to the recombinant OVA positive control.

The data emphasize that the immune response potential of the hybrid vector without the need for adjuvant inclusion and prior to optimization studies and that hybrid device signal strength when normalized to the amount of genetic antigen administered, highlighting the efficiency of the vector to invoke a response when compared to the positive control. The results further support the potential of the hybrid vector technology towards APC-mediated immune modulation and eventual full vaccination strategies.

The hybrid bio-synthetic vector developed in this study combined the capabilities of individual biological and biomaterial components. The new vector thus allowed 5 for a significantly expanded set of variables available to influence APC response and gene delivery. This was demonstrated through improvements to APC gene delivery, cytotoxicity, and NO formation when utilizing the base hybrid vector design.

Notably, the E. coli component of the hybrid vector allowed for a substantial improvement in gene delivery efficiency. Results were extended by leveraging the engineering capabilities of the hybrid vector to further address the cellular barriers to APC gene delivery. The new vector and approach offer a broader set of features and capabilities through which APC gene delivery can be modulated to carefully elicit desired immune responses.

### Example 10: Bacterial Hybrid Vectors Provide Protective Immunity to Pneumococcal Surface Protein A

### Methods

The scope of the hybrid bacterial vector was further developed uzing a pneumococcal vaccine model. The result of immunizing groups of mice with a model antigen, pneumococcal surface protein A [A], against pneumococcal infection that is not protective against challenge when administered via conventional means.

Specifically, mice were immunized at day 0 and 14, before being challenged via the intraperonital (i.p) route with a lethal dose of the bacteria on day 28.

### Results

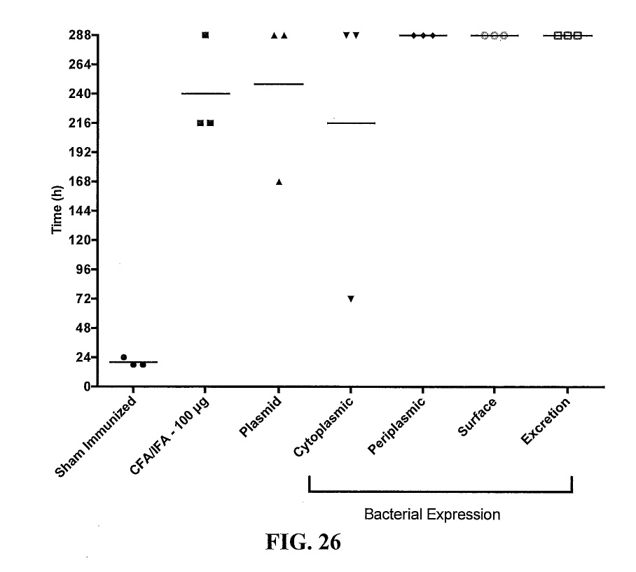

The data are presented in FIG. 26. Points of to note include the bacterial localization of PspA to various regions in the bacteria (e.g., cytoplasm, periplasm, surface, or secreted). In addition, the amount of PspA being delivered by the hybrid system is estimated to be between 100 and 10,000 fold lower than the 100 μg being delivered by the positive control.

Challenge with this amount of pathogen is noted for killing mice within 24-48, even when immunized with PspA (less than 10 μg).

1. A hybrid bacterial vector for delivery of polypeptides and nucleic acids to an eukaryotic cell comprising
a prokaryote cell and one or more biodegradable cationic polymers,
wherein the biodegradable cationic polymers are associated with the outer surface of the prokaryote cell in an amount sufficient to impart a positive charge to the prokaryote cell at physiological pH, and
wherein the prokaryote cell comprises one or more nucleic acid plasmids comprising
a. one or more genes encoding a polypeptide or nucleic acid sequence; and
b. one or more genes encoding a pore-forming protein or an endolysin enzyme.

2. The hybrid bacterial vector of claim 1 wherein the prokaryote cell is selected from the group consisting of a live, un-attenuated bacterium; a live, attenuated bacterium; and an inactivated bacterium.

3. The hybrid bacterial vector of claim 2 wherein the cell is a strain of Escherichia coli that is non-pathogenic in humans.

4. The hybrid bacterial vector of claim 3 wherein the Escherichia coli strain is selected from the group consisting of strain 1; strain S1 (YWT7-hly); Escherichia coli derivative B; Escherichia coli derivative K; BL21-DE3, Nissle 1917; W3110; DH5αE; Dam dcm strain; REL606; Escherichia coli strain C; Escherichia coli strain W; and genetically modified variants thereof.

5. The hybrid bacterial vector of claim 1, wherein one or more biodegradable cationic polymer is selected from the group consisting of poly(beta-amino esters); aliphatic polyesters; polyphosphoesters; poly(L-lysine) containing disulfide linkages; poly lactic acid; poly(ethylenimine); disulfide-containing polymers such as DTSP or DTBP crosslinked PEI; PEGylated PEI crosslinked with DTSP; Crosslinked PEI with DSP; Linear SS-PEI; DTSP-Crosslinked linear PEI; and branched poly(ethylenimine sulfide) (b-PETS).

6. The hybrid bacterial vector of claim 5 wherein one biodegradable cationic polymer is a poly(beta-amino ester).

7. The hybrid bacterial vector of claim 6 wherein the poly(beta-amino ester) is Acrylate-terminated poly(neopentyl glycol diacrylate-co-2-amino-1,3-propanediol).

8. The hybrid bacterial vector of claim 5 wherein one biodegradable cationic polymer is a cationic poly lactic acid.

9. The hybrid bacterial vector of claim 1 wherein one or more biodegradable cationic polymers are modified by the addition of a poly(alkylene oxide).

10. The hybrid bacterial vector of claim 9 wherein the biodegradable cationic polymer is modified by the addition of poly(ethylene glycol) having a molecular weight of between 100 Da and 16,000 Da, inclusive.

11. The hybrid bacterial vector of claim 1, wherein the charge density of the biodegradable cationic polymer is between −50 and −30 mV, inclusive.

12. The hybrid bacterial vector of claim 1, wherein the weight average molecular weight, as measured by gel permeation chromatography, is from about 900 Daltons to about 25,000 Daltons, inclusive, preferably from about 5,000 Daltons to about 6,000 Daltons, inclusive, most preferably approximately 5,500 Daltons.

13. The hybrid bacterial vector of claim 1, wherein the biodegradable cationic polymer includes one or more functional groups selected from the group consisting of targeting elements, immune-modulatory elements, chemical groups, biological macromolecules, or combinations thereof.

14. The hybrid bacterial vector of claim 13, wherein the biodegradable cationic polymer comprises an immune-modulatory element selected from the group consisting of CRM197 (diphtheria toxin), outer membrane protein complex from Neisseria meningitides, viral hemagglutinin and neuraminidase.

15. The hybrid bacterial vector of claim 13, wherein the biodegradable cationic polymer comprises a targeting element that is a ligand for a receptor selected from the group consisting of Fc gamma RIM; DCIR; DC-SIGN; Dectin-1; CLEC9A; Langerin; CD11c; CD163; FC gamma RIIB; or Her2.

16. The hybrid bacterial vector of claim 13, wherein the biodegradable cationic polymer comprises a targeting element that is an antibody, antibody fragment, or proteins having the binding specificity of an antibody.

17. The hybrid bacterial vector of claim 13, wherein the biodegradable cationic polymer comprises one or more targeting elements that mediate bacterial uptake by CD206.

18. The hybrid bacterial vector of claim 17, wherein the biodegradable cationic polymer targeting elements include one or more mannose moieties.

19. The hybrid bacterial vector of claim 1 comprising the pore-forming protein listeriolysin O.

20. The hybrid bacterial vector of claim 1, wherein the prokaryotic cell expresses the lethal lysis LyE gene of bacteriophage ΦX174.

21. The hybrid bacterial vector of claim 1 wherein the nucleic acid plasmid comprises
a. a promoter;
b. an exogenous nucleic acid downstream of and operably linked to the promoter;
c. a transcription terminator downstream of and operably linked to the exogenous nucleic acid; and
d. an origin of replication.

22. The hybrid bacterial vector of claim 21, wherein the promoter and transcription terminator elements are selected from the group consisting of viral, prokaryotic, eukaryotic and combinations thereof.

23. The hybrid bacterial vector of claim 22, wherein the promoter is an inducible promoter.

24. The hybrid bacterial vector of claim 1, wherein one exogenous nucleic acid is selected form the group consisting of ribozymes, enzymes, peptides, structural proteins, structural RNA, shRNA, siRNA, miRNA, transcription factors, and signaling molecules.

25. An adjuvant for stimulating an immune response to an antigen in a subject comprising the hybrid bacterial vector of claim 1,
wherein the one or more nucleic acid plasmids comprise one or more genes encoding the antigen.

26. The adjuvant of claim 25, wherein the antigen polypeptide is expressed within the prokaryotic cell at a location selected from the group consisting of the cytoplasm, the periplasm, the bacterial surface and combinations thereof.

27. The adjuvant of claim 25, wherein the antigen polypeptide is expressed in the prokaryotic cell and secreted from the cell.

28. A pharmaceutical composition comprising the adjuvant of claim 25 and a pharmaceutically acceptable excipient.

29. The pharmaceutical composition of claim 28, wherein the excipient is suitable for administration via the a route selected from the group consisting of oral, nasal, ocular, rectal, intramuscular, intraperitoneal, pulmonary, epidermal and intradermal.

30. The pharmaceutical composition of claim 28, further comprising a therapeutic, prophylactic or diagnostic agent.

31. A method for inducing or stimulating an immune response to an exogenous antigen in the antigen presenting cells of a subject comprising
administering to the subject the pharmaceutical formulation of claim 28 in an amount sufficient to induce an immune response in the antigen presenting cells of the subject.

32. The method of claim 31, wherein the subject is a mammal.

33. The method of claim 32, wherein the subject is a human.

34. The method of claim 31, wherein the subject is an avian.

35. The method of claim 31, wherein the antigen presenting cells are selected from the group consisting of dendritic cells, B cells, neutrophils and macrophages.

36. The method of claim 31, wherein
one or more exogenous antigens are selected from the group consisting of a viral antigen, a bacterial antigen, a protozoan antigen, a fungal antigen, a nematode antigen, a cancer antigen and combinations thereof.

37. A method for delivery of polypeptides and nucleic acids into an eukaryotic cell comprising
contacting the eukaryotic cell with the hybrid bacterial vector of claim 1 in an amount and concentration effective to facilitate uptake of the hybrid bacterial vector by the eukaryotic cell, wherein the hybrid bacterial vector causes minimal or no toxicity in the eukaryotic cell.
